(19) United States
(12) Patent Application Publication
(10) Pub. No.: US 2025/0205421 A1
(43) Pub. Date: Jun. 26, 2025

(54) METHOD AND APPARATUS FOR DETECTING IV INFILTRATION AND MANAGING INTRAVENOUS FLUID DELIVERY

(71) Applicant: Eduardo Rodrigues, Ponte Vedra, FL (US)

(72) Inventor: Eduardo Rodrigues, Ponte Vedra, FL (US)

(21) Appl. No.: 19/001,487

(22) Filed: Dec. 25, 2024

Related U.S. Application Data
(60) Provisional application No. 63/614,908, filed on Dec. 26, 2023.

(51) Int. Cl. A61M 5/168; A61M 5/14; A61M 5/145; A61M 5/36; G16H 40/63

(52) U.S. Cl. CPC A61M 5/16854; A61M 5/1407; A61M 5/1452; A61M 5/16804; A61M 5/16886; A61M 5/365; G16H 40/63; A61M 2005/16863; A61M 2202/0007; A61M 2202/04; A61M 2205/11; A61M 2205/18; A61M 2205/3327; A61M 2205/3334; A61M 2205/3344; A61M 2205/3368; A61M 2205/3375; A61M 2205/3553; A61M 2205/3561; A61M 2205/3576; A61M 2205/505; A61M 2205/52; A61M 2205/583

(57) ABSTRACT

An apparatus and method for detecting intravenous (IV) infiltration and managing fluid delivery in an IV setup are disclosed. The apparatus includes a controller with an artificial intelligence (AI) engine to process real-time and historical IV parameters, a communication module for remote monitoring and control, and a sensing arm with sensors to monitor parameters like pressure, flow rate, temperature, acoustic signals, and air bubbles within the IV line. A first clamp and a second clamp are attachable to the IV line to selectively occlude fluid flow. Syringe holders connected to the IV line via an aggregation tube or Luer lock include a plunger control mechanism for adjustable injection timing, pressure, and volume. Monitored parameters are analyzed to detect IV infiltration, blockages, or abnormalities, and alerts are transmitted to medical professionals via the communication module or displayed on an integrated user interface, enabling real-time detection and dynamic adjustments.

### CROSS REFERENCE TO RELATED APPLICATION

The present application claims priority to U.S. Provisional Application No. 63/614,908 filed Dec. 26, 2023, entitled METHOD AND APPARATUS FOR DETECTION OF IV INFILTRATION, the entire disclosure of which is incorporated herein by reference.

### FIELD OF THE INVENTION

The present invention relates to medical devices and methods for intravenous (IV) therapy. Specifically, it pertains to an apparatus and method for detecting IV infiltration and managing fluid delivery through an IV setup. The invention integrates real-time monitoring, artificial intelligence (AI)-driven analysis, and automated control of IV parameters, including pressure, flow rate, temperature, and acoustic signals. The apparatus is configured to identify irregularities such as IV infiltration, blockages, and air bubbles, while enabling dynamic adjustments to fluid injection timing, pressure, and volume. It further supports remote monitoring, alert transmission, and data management to enhance patient safety and improve the efficiency of IV therapy.

### BACKGROUND OF THE INVENTION

Intravenous (IV) therapy is one of the most widely used medical procedures globally. Approximately 300,000,000 peripheral IVs are placed in the US every year. It is the most common invasive medical procedure performed worldwide. It is a cornerstone of modern healthcare, enabling the direct administration of fluids, medications, and nutrients into a patient's bloodstream. It is estimated that one billion peripheral IVs are inserted annually worldwide. Despite its critical importance, the procedures and methods associated with IV therapy remain prone to complications, which can result in significant patient harm, prolonged treatment times, and increased healthcare costs. Peripheral IVs are known to malfunction 30 to 50% of the time during their intended use.

IV therapy commonly involves the insertion of a peripheral intravenous (PIV) catheter into a vein, typically in the patient's arm or hand. The catheter is then connected to a source of fluid or medication, such as a saline solution, glucose pouch, or medication-filled bottle. Fluids and medications are delivered into the bloodstream either by gravity or through infusion pumps, which regulate flow rates. Despite the relative simplicity of these systems, IV therapy carries inherent risks and challenges.

Conventional methods for managing IV therapy depend heavily on manual procedures and subjective assessments performed by healthcare professionals. During the administration of fluids and medications, practitioners are required to observe the IV site visually and palpate the surrounding area to ensure proper functioning. This process often involves assessing for swelling, redness, pain, or resistance during infusion. However, these techniques are qualitative in nature and rely significantly on the experience and vigilance of the medical provider.

One of the most common complications in IV therapy is IV infiltration, which occurs when the catheter becomes dislodged or misplaced, allowing fluid to leak into surrounding tissues rather than entering the bloodstream. In such cases, the intended therapeutic effects of medications or fluids are not achieved, and the patient may experience swelling, pain, and tissue damage. In severe cases, infiltration can result in compartment syndrome, which requires surgical intervention, or tissue necrosis, which can lead to permanent damage.

Another serious complication is extravasation, a form of infiltration involving vesicant medications, such as chemotherapy drugs or calcium-based solutions, which can cause severe tissue injury when leaked into surrounding tissues. These events often necessitate emergency interventions, such as tissue debridement, and can result in long-term physical harm and emotional distress for the patient.

In addition to infiltration and extravasation, occlusion is another common problem associated with IV therapy. Occlusions can occur when the IV line becomes blocked due to blood clots, medication precipitates, or mechanical kinking of the tubing. Occlusion disrupts the flow of fluids or medications and requires intervention to restore proper function, often leading to delays in treatment.

Other potential risks in IV therapy include air embolism, where air bubbles in the IV line enter the bloodstream, potentially causing life-threatening complications, and line disconnection, which can result in fluid leakage or contamination. These complications often go unnoticed until they have progressed significantly, posing serious risks to patient safety.

In cases where the peripheral IV catheter may become dislodged and located not inside the peripheral vein as intended but in the subcutaneous tissue, the intended intravascular fluid administration fails to achieve systemic circulation, emergency medications may not be effective, and tissue damage may occur due to pressure generated by the infiltrated volume, due to compartment syndrome, or due to toxicity from the medication. Some exemplary medications which may be known to cause severe tissue damage, like calcium citrate, Phenergan, Chemotherapy medications, and bicarbonate may be administered during an aberrant intravenous procedures.

Therefore, when IVs are aberrant, infiltrated IVs frequently cause patient harm, dissatisfaction, and health care liability. Support apparatus and methods are needed to assist healthcare professionals in detecting intravenous (IV) catheter malposition.

Efforts have been made to address these challenges through the development of improved materials, infusion pumps, and specialized training programs for healthcare professionals. Modern infusion pumps, for example, can regulate flow rates with high precision and alert providers to issues like flow resistance or air in the line. However, these pumps lack the ability to provide detailed diagnostic feedback, such as identifying whether resistance is caused by a kink in the tubing or by infiltration into surrounding tissues. Similarly, while advanced catheter materials and designs have reduced some risks, they do not eliminate the possibility of complications.

The current standard method for assessment of whether an IV is properly functioning involves a subjective assessment based on vigilance and experience of an experienced medical provider to detect when the PIV is infiltrated. The assessment procedure may involve the medical provider directly palpating and perceiving the state as well as observing the PIV region. The qualitative and subjective nature of these procedures frequently results in misdiagnoses and medications being administered subcutaneously. Even experienced providers may often fail to detect infiltrated IVs.

Healthcare providers often rely on their experience and subjective judgment to identify and respond to IV complications. This dependence on manual assessments introduces variability and increases the likelihood of errors, particularly in high-pressure or resource-limited environments. Vulnerable populations, such as pediatric and elderly patients, are at even greater risk due to their smaller veins, thinner skin, and reduced ability to communicate discomfort effectively. As a result, healthcare professionals face significant challenges in ensuring the safe and effective delivery of IV therapy.

Despite the availability of some advanced tools, there remains a critical gap in real-time monitoring and automated detection of IV therapy complications. For example, current methods for detecting infiltration typically involve observing for visible swelling or patient-reported pain, which are subjective indicators and often manifest only after significant tissue damage has occurred. Similarly, the detection of occlusions or air bubbles is often delayed until the infusion pump triggers an alarm, by which time corrective action may be more complex and invasive.

In summary, while IV therapy is an essential component of medical care, it is associated with a range of complications that can compromise patient safety and increase healthcare costs. Conventional methods and tools used for IV therapy management rely heavily on subjective assessments, manual monitoring, and reactive interventions, leaving significant room for improvement in both accuracy and efficiency. There is a pressing need for systems and methods that can provide objective, real-time monitoring of IV therapy, enabling healthcare providers to detect and address complications proactively, reduce the risks associated with IV therapy, and improve patient outcomes.

### SUMMARY OF THE INVENTION

Accordingly, methods and apparatus are described to support the detection of a state of IV infiltration utilizing sensors and automation to measure an introduced volume of fluid through an installed IV and detect aberrant conditions.

In some examples, a method of detecting a state of IV infiltration may include a step of installing an IV into a patient. The method may further include connecting an IV line to a primary input connection of a first device, wherein the first device supports the detection of a state of IV infiltration. The method may further include connecting an IV line from the installed IV to an output connection of the first device to support the detection of a state of IV infiltration. The method may further include placing a syringe holder upon a secondary input connection of the first device. The method may further include staging the syringe holder to hold a spring in one of a compression or expansion state. In some examples, the method may include placing a syringe with a fluid solution within the syringe holder. The method may further include monitoring at least one of pressure, temperature, or flow rate with sensors of the first device, The method may further include releasing an engagement arm on the syringe holder to pressurize the fluid solution within the syringe holder. The method may further include recording the sensed and monitored parameters. The method may further include processing the monitored parameters; and providing feedback to a user based on the processing of the monitored parameters.

These methods may include examples where the first device (IV infiltration detection apparatus) further comprises a processor configured to execute software instructions, process real- time and historical data, and analyze sensor inputs to detect deviations indicative of IV infiltration, blockages, or abnormalities in fluid flow, thereby enabling dynamic adjustments to operational parameters such as flow rate, pressure, and timing.

These methods may include examples wherein the first device further comprises a display configured to visually present real-time data, including monitored parameters such as pressure, flow rate, and temperature, as well as alerts and notifications related to IV infiltration or irregularities, and to allow users to input and adjust operational settings.

These methods may include examples wherein the first device comprises a pressure sensor operably connected to the IV line, configured to monitor pressure variations in the IV line indicative of blockages, infiltration, or reverse pressure caused by backflow of blood due to patient movement or other factors.

These methods may include examples wherein the first device comprises a fluid flow sensor, such as an ultrasonic flow meter, configured to detect fluid flow rate and irregularities within the IV line, enabling precise detection of anomalies such as restricted or excessive fluid flow conditions.

These methods may include examples wherein the first device comprises a temperature sensor, operably integrated with the sensing mechanisms, to detect deviations in fluid temperature that may indicate abnormalities in fluid delivery or environmental influences on the IV setup.

These methods may include examples wherein the first device comprises an electrical timing circuitry configured to regulate injection timing, synchronize sensor operations, and maintain time-accurate data logging, ensuring precise monitoring and control of fluid delivery within the IV setup.

In some examples, apparatus for detecting a state of IV infiltration may include a primary input connection, a secondary input connection, and an output connection. The apparatus may further include one or more sensing elements. The apparatus may further include a primary chamber to interface with the primary input connection and the secondary input connection, and the output connection and the one or more sensing elements. In some embodiments, the apparatus may further include a vessel to contain a testing volume of a fluid for a forced flow through the output connection.

In some embodiments, this apparatus may include examples wherein the vessel comprises a syringe, configured to hold a predetermined volume of fluid for injection into the IV line, the syringe being operably connected to a plunger mechanism for controlled fluid delivery, adjustable in timing, volume, and pressure.

In some embodiments, this apparatus may include examples wherein the vessel comprises an elastic membrane, designed to hold and release fluid under controlled pressure, the elastic membrane being operably configured to deform in response to compression or expansion forces for precise fluid delivery.

embodiments In some examples, the apparatus may include examples wherein the means to compress the testing volume of the fluid comprises a spring, the spring being adjustable to regulate the force applied to the vessel, thereby enabling fine control over the rate and pressure of fluid delivery into the IV line.

In some embodiments, the apparatus may include examples wherein the means to compress the testing volume of the fluid comprises a compressed fluid, such as air or gas, used to exert pressure on the vessel, enabling consistent and adjustable fluid injection for therapeutic or diagnostic applications.

In some embodiments, the apparatus may further include one or more of a check valve and a controlled valve, the check valve being configured to allow unidirectional fluid flow, and the controlled valve enabling precise regulation of fluid flow rates and volumes. The apparatus may further include a pumping means, such as an electric pump, operably connected to the vessel to generate consistent fluid flow or injection pressure. The pumping means may include an infusion pump configured to deliver precise volumes of fluid at predetermined rates and pressures. The infusion pump may further incorporate adjustable settings controlled via the apparatus's processor, enabling customization of injection parameters such as flow rate, timing, and pressure based on therapeutic requirements. Additionally, the infusion pump may integrate feedback from sensing elements to dynamically adapt to real-time data, such as pressure deviations or detected abnormalities, ensuring accurate and reliable fluid delivery.

In some embodiments, the apparatus may further include a processor configured to execute software instructions, process sensor data, and control the various components of the apparatus, including valves, pumps, and sensing mechanisms, to optimize fluid delivery and detect abnormalities such as IV infiltration or blockages.

There may be methods of detecting a state of IV infiltration including providing a primary input connection of a first device, wherein the first device supports the detection of a state of IV infiltration. The methods may further include providing an output connection of the first device. The methods may further include placing a vessel upon a secondary input connection of the first device. The methods may further include monitoring at least one of pressure, temperature, or flow rate with sensors of the first device. The methods may further include activating a release of pressure within the vessel to engage a flow of fluid through the output connection of the first device. The methods may further include recording the sensed and monitored parameters. The methods may further include processing the monitored parameters; and providing feedback to a user based on the processing of the monitored parameters.

In some embodiments, the method may include examples wherein the processing of the monitored parameters is performed by a processor of the first device. In some other embodiments, the method may include transmitting the data of the monitored parameters to an external host, and processing data of the monitored parameters at the external host. In still further embodiments, the method may include processing of the monitored parameters at least in part, and may comprise processing data with an artificial intelligence algorithm which has been trained with data collected from apparatus of the same type as the first device.

### BRIEF DESCRIPTION OF THE DRAWINGS

FIGS. 1A-1B illustrate an exemplary methodology according to normal practice for installing an IV into a patient.

FIGS. 2A-2C illustrate exemplary apparatus to support medical professionals for detection of IV infiltration from different viewpoints.

FIGS. 2D-2F illustrate exemplary applications of the apparatus connected to an intravenous (IV) line, showing configurations where the IV line is connected to a fluid source at one end and to a cannula inserted into a patient's vein at the other end.

FIG. 3 illustrates components that may be included in an exemplary apparatus to support medical professionals for detection of IV infiltration.

FIG. 3A illustrates a block diagram showcasing exemplary internal components of the apparatus in some embodiments of the present disclosure.

FIG. 4A illustrates components of a syringe for use in the apparatus in some implementations of the present disclosure.

FIG. 4B illustrates a syringe, syringe holder and apparatus to support medical professionals for the detection of IV infiltration in an assembled fashion.

FIG. 4C illustrates an exemplary vessel and apparatus to support medical professionals for the detection of IV infiltration in an assembled fashion.

FIG. 4D illustrates an apparatus to support medical professionals for the detection of IV infiltration with an included vessel as part of the apparatus.

FIG. 4E illustrates a compact form of the apparatus with a syringe, configured to support medical professionals in detecting IV infiltration, shown in an assembled fashion connected to an IV line.

FIG. 4F illustrates the apparatus with clamps connected to the IV line to control fluid flow, along with a syringe for administering fluids or medication.

FIG. 5 illustrates exemplary method steps according to the present disclosure for detecting IV infiltration according to some examples of the present disclosure.

FIG. 6 illustrates additional exemplary method steps according to the present disclosure for detecting IV infiltration according to some examples of the present disclosure.

FIG. 7 illustrates a combined apparatus with a single syringe integrated into an infusion pump and IV infiltration detection setup, connected to an IV line for administering fluids and monitoring flow conditions.

FIG. 8 illustrates an apparatus featuring an infusion pump with multiple syringe holders integrated with an IV infiltration detection setup, connected to an IV line for administering fluids and monitoring flow conditions.

FIG. 9 illustrates the exemplary working of the clamps within the apparatus and their integration with the IV infiltration detection setup, connected to an IV line for monitoring and fluid administration.

FIG. 10 illustrates the apparatus with an internal fluid flow line connected to input-output ports which are further connected to the IV line, and the clamps and sensing elements are attached to the internal fluid flow line.

FIG. 11 illustrates the apparatus where the IV line itself passes through the apparatus, with clamps and sensing elements internally attached to the IV line.

FIG. 12 illustrates a system configured to enable remote control of the apparatus by a medical professional, with notifications and commands transmitted through a connected device.

FIGS. 13A-13C illustrate exemplary tables showcasing various control parameters, sensing mechanisms, operational features, and data outputs for the IV infiltration detection device.

FIGS. 14A-14B illustrate additional method steps that may be executed in some implementations of the present disclosure.

### DETAILED DESCRIPTION OF THE INVENTION

The present invention provides for methods and apparatus to facilitate detection of intravenous (IV) infiltration, dislodged, or other catheter malposition. A failed IV may be located in subcutaneous tissue, muscle, or other area that is not inside an intended peripheral vein. A mispositioned IV may result in intended intravascular fluid administration failing to achieve systemic circulation, emergency medications will not be effective, and tissue damage can occur from pressure generated by the infiltrated volume, due to compartment syndrome, or due to toxicity from the medication. Some medications which may be known to cause severe tissue damage include, by way of non-limiting example, one or more of: calcium citrate, Phenergan, Chemotherapy medications, and bicarbonate.

According to some estimates, approximately 300,000,000 peripheral IVs are placed in the United States every year and between 90,000,000 to 150,000,000 of those malfunction. The present invention provides for pragmatic detection of when a peripheral IV (PIV) mal-functions due to mispositioning, or are otherwise non-optimal.

In some embodiments of the present invention, an apparatus is provided to detect a state of IV infiltration in an IV setup and manage fluid delivery with precision and adaptability. The apparatus integrates advanced monitoring, analysis, control, and communication capabilities to support real-time decision-making and improve patient safety.

The apparatus includes a controller comprising a processor and an artificial intelligence (AI) engine. The controller executes software instructions to process data collected in real-time, analyze historical IV parameters, and manage operational components such as clamps, sensing arms, and syringe mechanisms. For example, the controller can analyze pressure, temperature, and acoustic feedback from sensors to identify patterns indicative of IV infiltration or fluid blockages.

The communication module may be operably connected to the controller and supports data transmission and reception through Wi-Fi, Bluetooth, or cellular connectivity. This module enables alerts and data to be sent to medical professionals' devices, such as smartphones or tablets, so that they remain informed of IV line conditions. Additionally, the communication module allows remote commands to be sent back to the apparatus, enabling medical professionals to adjust clamp positions, syringe operations, or flow rates without physical interaction with the device. For example, during a detected irregularity, the communication module can receive a command to increase fluid injection pressure or activate flushing operations.

A data storage module records sensor data, operational parameters, and event logs, including timestamps for compliance, analysis, and record-keeping. For example, historical data on pressure variations can be analyzed by the AI engine to generate predictive alerts, such as warnings about potential IV infiltration risks based on trends.

The sensing arm may be attachable to an IV line and comprises multiple sensors, including pressure, temperature, acoustic, and air bubble detection sensors. These sensors monitor parameters such as fluid flow rate, temperature deviations, and acoustic signals. For example, the acoustic sensor can detect sound wave patterns indicative of air bubbles, while the temperature sensor identifies abnormal fluid temperature changes. The sensing arm continuously sends data to the controller for analysis, enabling real-time feedback.

The apparatus features a first clamp and a second clamp, each operably connected to the controller and attachable to the IV line at proximal and distal points, respectively. The first clamp, positioned near the fluid source, selectively occludes fluid flow during syringe filling or in response to detected irregularities, such as blockages or high-pressure spikes. The second clamp, positioned near the patient's cannula, controls fluid flow during injection or when IV infiltration is suspected. Both clamps are constructed with medical-grade silicone-lined surfaces to prevent damage to the IV line during operation and are controlled through hydraulic lines for precise adjustment based on real-time sensor data.

The apparatus includes one or more syringe holders designed to accommodate syringes of varying sizes. The syringes are connected to an aggregation tube, which merges multiple fluid sources before connecting to the IV line via a Luer lock. The syringe holders integrate with a plunger control mechanism that adjusts the syringe plunger's movement for variable injection volumes and timings. For example, a medical professional can remotely configure the plunger to deliver 5 mL of medication over a period of 10 seconds via commands transmitted through the communication module.

An internal fluid flow line may be integrated within the apparatus, connecting an input port (linked to the IV line from the fluid source) and an output port (linked to the IV line leading to the patient). Components such as the sensing arm, first clamp, and second clamp are internally attachable to this flow line, enhancing sterility and reducing external exposure.

To maintain hygiene, the apparatus features a UV sanitization system that automatically sanitizes the internal fluid flow line, syringe holders, syringes, and clamps before and after use. This system reduces the risk of contamination and facilitates compliance with medical standards.

The apparatus includes a display means with a user interface, operably connected to the controller. The display provides real-time visualizations of IV parameters, including pressure, flow rate, and temperature. It also allows users to configure operational parameters, such as injection volume and timing, and provides visual alerts for IV infiltration or abnormalities. In one embodiment, the display may be a touchscreen interface, enabling intuitive interaction with the device.

The apparatus may be equipped with advanced alert and feedback mechanisms. For example, the controller can generate alerts upon detecting irregularities like air bubbles or blockages. These alerts are transmitted via the communication module to medical professionals' devices. Additionally, the apparatus supports predictive alerts generated by the AI engine, enabling early intervention before severe infiltration occurs.

The first clamp and the second clamp may include integrated sensors to monitor micro-pressure variations, providing detailed feedback on IV line conditions. For example, these sensors can detect slight resistance changes indicative of blockages or infiltration at the proximal or distal end of the IV line.

The apparatus further supports patient-specific customization. The controller can execute software instructions to dynamically adjust flow rates based on therapy protocols stored in the data storage module. For example, pediatric patients requiring lower flow rates and pressures can be accommodated by configuring the apparatus accordingly.

The communication module enables seamless integration with external devices and systems. It allows medical professionals to send commands to adjust injection timing, pressure, or fluid volume remotely. In one scenario, a nurse may activate an emergency injection of a life-saving drug by sending a command through a smartphone app linked to the apparatus.

Finally, an activation element may allow on-site staff to manually send alerts to medical professionals. For example, pressing the activation element during an emergency transmits a high-priority notification, prompting immediate action.

In some embodiments, a method is provided for detecting IV infiltration and managing fluid delivery through an IV setup using an advanced apparatus that integrates clamps, a sensing arm, syringe holders, and communication capabilities. This method facilitates real-time monitoring, analysis, and control of IV therapy, enhancing patient safety and clinical efficiency.

The process begins by connecting a first clamp of the apparatus to the IV line at a proximal point near the fluid source. This clamp is configured to selectively occlude the IV line to regulate fluid flow during syringe filling or to respond to irregularities detected in the IV line. For example, if the fluid source is a saline bag, the first clamp can be closed to isolate the fluid flow when medication from a syringe is being administered.

Simultaneously, a second clamp is connected to the IV line at a distal point near the cannula inserted into the patient. This clamp operates in coordination with the first clamp to manage fluid delivery. For example, during fluid injection, the second clamp opens while the first clamp closes to direct medication from the syringe toward the patient.

A sensing arm is then attached to the IV line to monitor important parameters such as pressure, flow rate, temperature, acoustic resistance, and the presence of air bubbles. The sensing arm houses multiple sensors, each designed for specific monitoring tasks. For example, a pressure sensor detects sudden spikes or drops indicative of infiltration or blockages, while an acoustic sensor analyzes sound wave patterns to identify air bubbles or irregular fluid movement.

The data collected by the sensing arm may be continuously analyzed by a controller within the apparatus. The controller, equipped with a processor and an artificial intelligence (AI) engine, compares real-time data to baseline or historical IV parameters stored in the system. For example, the AI engine may recognize a gradual increase in resistance in the IV line as an early sign of infiltration, prompting further investigation or adjustments.

The monitored parameters, including sensor readings, timestamps, and logged events, are recorded in a data storage module for subsequent analysis. This recorded data can be used for compliance reporting, patient therapy optimization, or as a reference for medical professionals in diagnosing issues related to IV therapy.

If the analysis identifies an irregularity, such as the presence of air bubbles or abnormal pressure, the controller generates an alert. This alert may be transmitted via a communication module to an external device, such as a smartphone or tablet belonging to a medical professional. Alternatively, the alert may be displayed on an integrated user interface of the apparatus, providing real-time feedback on the detected irregularity.

The apparatus further supports receiving remote commands via the communication module, enabling medical professionals to adjust operational parameters such as clamp positions, injection timing, injection volume, or the activation of the sensing arm. For example, a nurse monitoring the system remotely may send a command to close the first clamp and open the second clamp to facilitate immediate medication delivery.

To facilitate fluid administration, one or more syringe holders are positioned on a secondary input connection of the apparatus. These holders securely accommodate syringes of varying sizes, which are operably connected to the IV line through an aggregation tube or a Luer lock. For example, a 5 ml syringe containing antibiotics and a 10 ml syringe with saline solution may be used in tandem for patient treatment.

Fluid injection is performed by activating a plunger control mechanism associated with the syringe holders. This mechanism, controlled by the apparatus's controller, regulates the plunger's movement for precise injection timing, pressure, and volume. For example, a medical professional may configure the apparatus to deliver 3 ml of medication over 10 seconds at a specified pressure to provide patient comfort and accuracy.

The method includes adjusting the first clamp and the second clamp to manage fluid flow during syringe filling and injection. For example:

The first clamp closes, and the second clamp opens during fluid injection to direct the medication toward the patient.
Conversely, the first clamp opens, and the second clamp closes during syringe filling to allow fluid from the source to refill the syringe.

After each injection, post-injection signals are detected using the sensing arm to evaluate fluid flow consistency, pressure feedback, and acoustic signals. For example, consistent acoustic patterns and pressure stability post-injection indicate successful fluid delivery, whereas deviations may suggest partial blockages or infiltration.

The controller dynamically adjusts injection timing, pressure, and volume based on real-time data analyzed by the AI engine. For example, if the AI engine detects increasing resistance in the IV line, it may reduce the injection pressure to prevent infiltration or tissue damage.

The recorded parameters are transmitted via the communication module to a centralized hospital information system for compliance and further analysis. This capability provides seamless integration of the apparatus into hospital workflows, supporting data-driven decisions and long- term patient care.

The method supports transmitting alerts via Wi-Fi connectivity, so that medical professionals are promptly informed of irregularities, even in remote settings. Additionally, the sensing arm's acoustic sensor analyzes sound wave patterns to detect air bubbles or irregular fluid flow, generating actionable alerts for quick intervention.

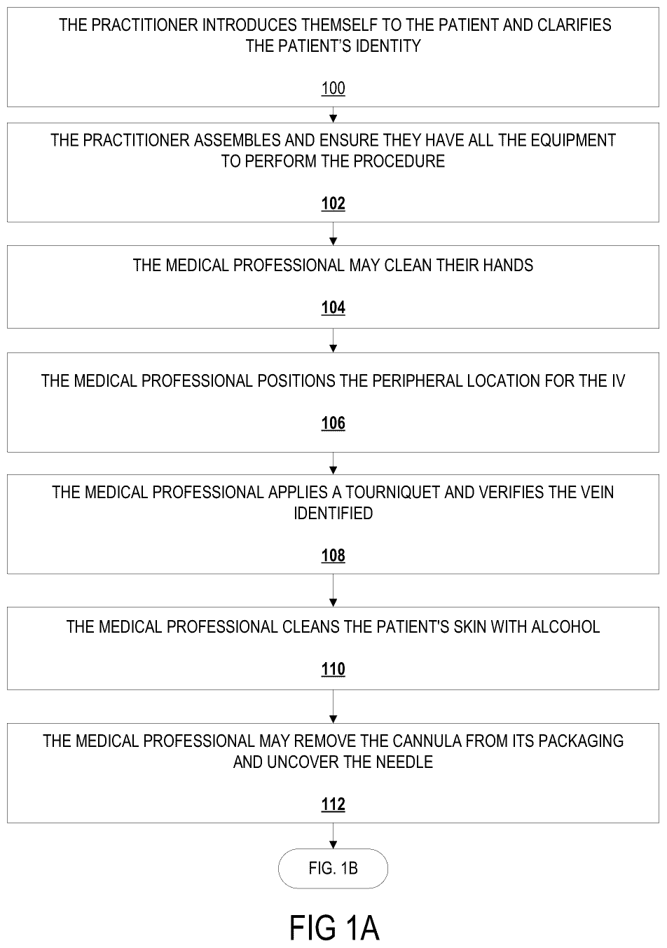

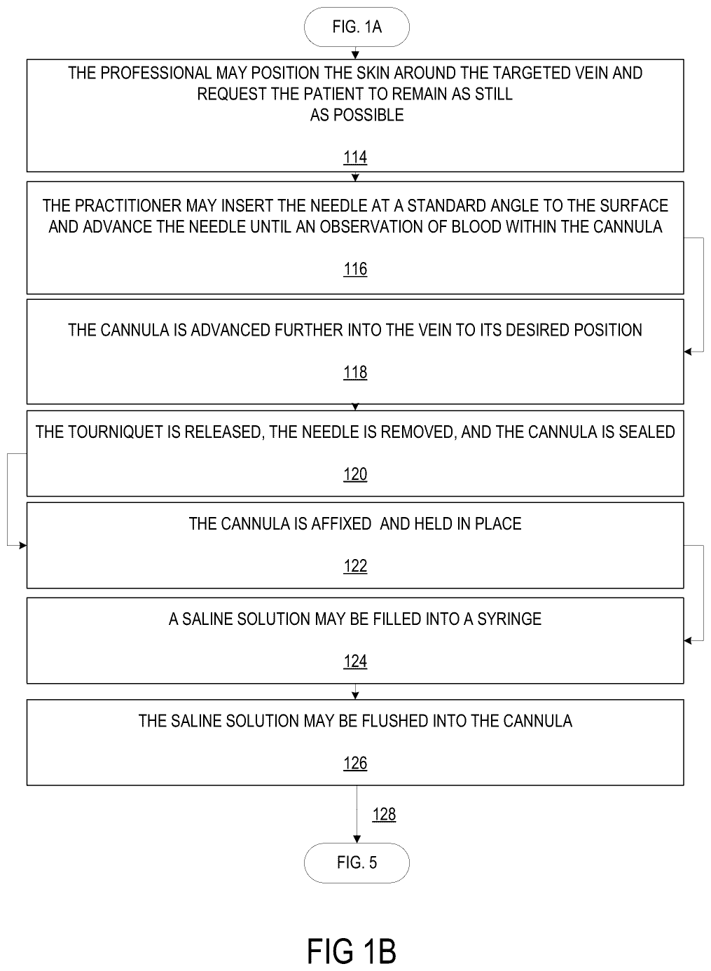

Referring to FIGS. 1A-1B, basic steps that a medical practitioner may follow to install an intravenous catheter may include: at step 100, the practitioner may introduce themself to the patient and clarify the patient's identity. The practitioner may explain an imminent procedure to the patient, and as appropriate, gain informed consent from the patient to continue with the procedure. The explanation may include informing the patient that cannulation may cause some discomfort.

At step 102, the practitioner may assemble and ensure that all equipment useful in performing the procedure is present, such as: gloves, tourniquets, cleaning wipes, alcohol, bandages, tapes, an IV cannula, syringes, saline solution, and the like.

At step 104 the practitioner may clean their hands, and at step 106, the practitioner may identify a peripheral location and position for the IV so that the IV is comfortable and presents a vein.

At step 108, the medical practitioner may apply a tourniquet and verify the vein identified.

At step 110 the medical practitioner may clean the patient's skin with alcohol.

At step 112, the medical practitioner may remove the cannula from its packaging and uncover the needle. At step 114, the practitioner may position the skin around the targeted vein and request the patient to remain as still as possible when the patient might feel pain during the procedure.

At step 116, the practitioner may insert the needle at a standard angle to the surface and advance the needle until an observation of blood within the cannula.

Referring now to FIG. 1B, at step 118, the cannula may be advanced further into the vein to its desired position. Proceeding at step 120, the tourniquet may be released, the needle may be removed, and the cannula may be sealed. At step 122 the cannula may be affixed and held in place with adhesive tape or other such holding material. Next, at step 124, a saline solution may be filled into a syringe. At step 126, the saline solution may be flushed into the cannula. It is at this point that the medical professional may typically observe if there is any resistance to the flow of the saline solution. The medical professional may also observe whether any swelling occurs in the region of the IV, and if any aberration is observed, the cannula may be removed, and the process may start again.

Even when procedures such as described above are followed, the reality is that some 30-50% of the IV processes may result in a poorly functioning IV being set up and used with deleterious effects. Accordingly, additional support apparatus may be useful in improving the procedures and improving outcomes of the procedures and other processing flow 128 may result in some of these examples.

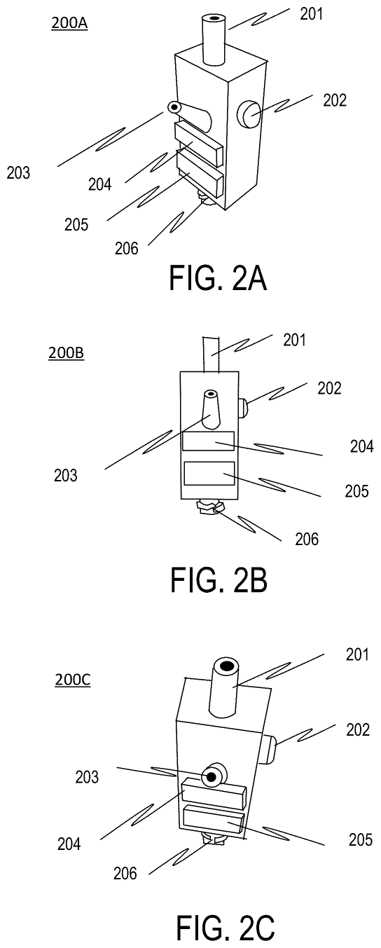

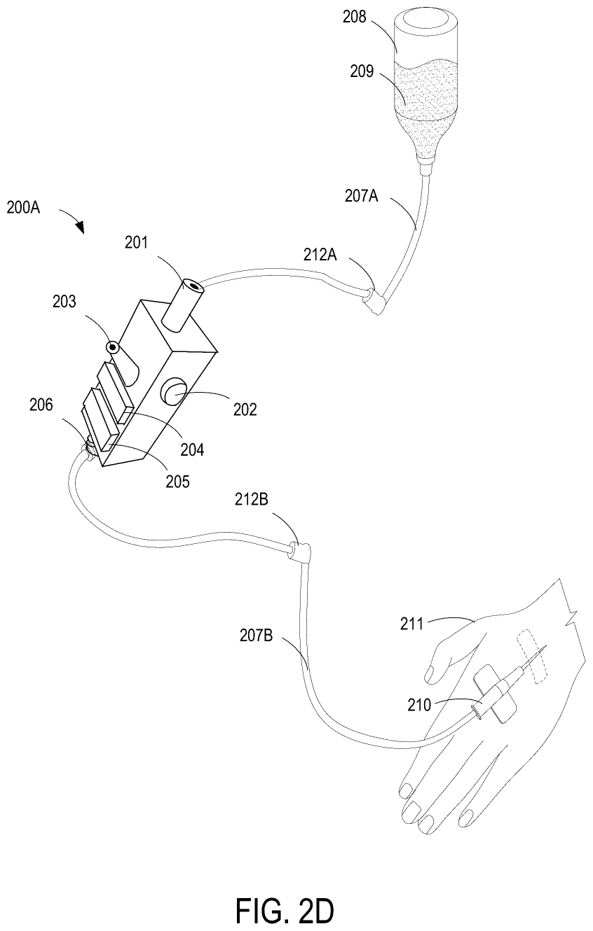

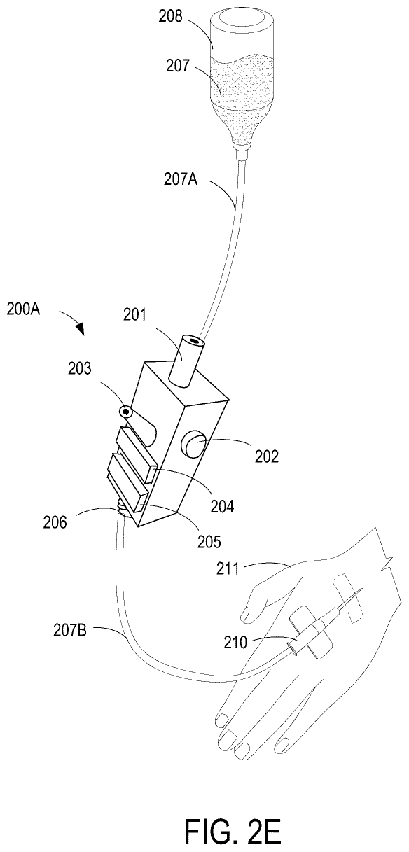

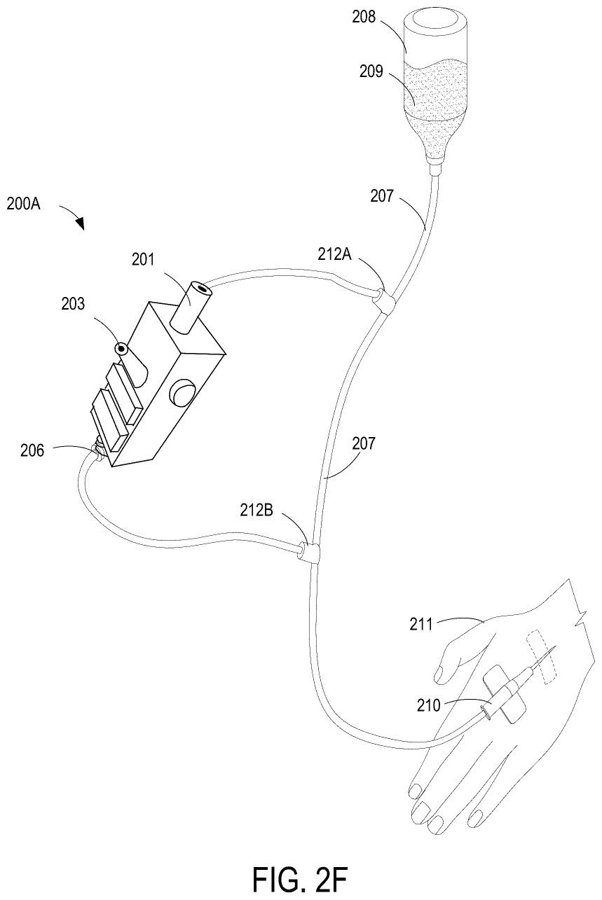

Referring now to FIG. 2A, FIG. 2B, and FIG. 2C, an exemplary apparatus to support detection of IV infiltration are illustrated. The apparatus may be coupled with a specialized syringe structure that allows saline or other fluid to be introduced into the secondary input connection 203 under controlled conditions.

Referring now to FIG. 2A, a perspective view is illustrated with exemplary components from an apparatus 200A to support the detection of IV infiltration including a primary connection to input tubing 201 (primary input connection) which may include standard locking elements such as Luer fitting, a Clave™ fitting, ridged connections, or other similar such sealing devices.

The apparatus 200A may also include an activation element 202 which may be a switch, push button, proximity detector, or the like. In some examples, activation may also be possible with a remote communication between an external device, such as a smart device, and the apparatus 200A to support the detection of IV infiltration (the “Apparatus”).

In some embodiments, the activation element 202 may be a manually operated switch configured to send an alert to a medical professional. For example, during intravenous therapy, a caregiver or patient may detect an issue such as irregular flow, infiltration symptoms, or patient discomfort. Activating the switch immediately transmits an alert signal to a connected monitoring system or directly to the medical professional's device via communication protocols such as Bluetooth, Wi-Fi, or cellular networks. This alert may include real-time data collected by the apparatus, such as pressure readings or flow rate anomalies, enabling the medical professional to assess the situation promptly and take corrective actions.

In other examples, the activation element 202 may be implemented as a touch-sensitive button, a voice-activated command input, or a proximity-based sensor. For example, a touch-sensitive button may allow for intuitive operation even in low-light conditions, while a voice-activated system enables hands-free use in sterile environments. Alternatively, a proximity sensor integrated into the activation element 202 may detect a hand gesture or movement to trigger an alert, offering a hygienic solution in scenarios where physical contact with the apparatus should be minimized. These alternatives provide flexibility and adaptability, so that the activation element 202 meets the needs of diverse medical environments and user preferences.

The apparatus 200A to support detection of IV infiltration may include a secondary input connection 203 which may be used to introduce fluids into the system for use in performing the diagnostic function of the apparatus 200A to support detection of IV infiltration. In some examples, the secondary input connection 203 may be capped when not in use. In some other examples, the secondary input connection 203 may include a check valve.

The apparatus 200A to support detection of IV infiltration may include a display means 204. In some examples, the display means 204 may be a screen such as an LCD, LED, or other type of display screen. In some other examples, the display means 204 may be a series of lights that indicate state. In some examples, the display means 204 may include a power source integral with the device (200A) which may include a wireless charging capability. In some other examples, the display means 204 may include a wireless communication capability such as Bluetooth radio in a non-limiting example, where display aspects may be communicated to display systems such as smart devices, display screens, computers, and the like. The display means 204 may comprise a user interface configured to display real-time data, including pressure, flow rate, and temperature, allow a user to configure operational parameters, such as injection volume, timing, and clamp operations, and provide visual alerts and notifications upon detecting IV infiltration or abnormalities.

The display means 204 in the apparatus 200A may be a multi-functional interface designed to provide real-time feedback and facilitate interaction with the apparatus. In some embodiments, the display means 204 may be a touch screen, allowing medical professionals to directly adjust the settings of the apparatus, such as flow rate, pressure, or infusion time. This touch screen may also include a user-friendly graphical interface displaying intuitive controls, such as sliders or buttons, for seamless configuration of parameters. For example, during an infusion, a medical professional may use the touch screen to modify the rate of medication delivery in response to a patient's condition.

In other embodiments, the display means 204 may be a combination of an LCD or OLED screen coupled with physical input controls, such as rotary dials or push buttons. This configuration can provide a more tactile interface for environments where gloves are required, such as surgical settings. The display may show useful data, including real-time pressure readings, remaining infusion volume, and alerts for anomalies like air bubbles or infiltration. In yet another embodiment, the display means 204 may integrate voice or gesture recognition technology, enabling hands-free operation for added convenience in sterile environments.

The apparatus 200A to support detection of IV infiltration may include a control and sensing system 205. In some examples, the control and sensing system 205 may include elements such as processors, pressure sensors, temperature sensors, flow sensors, check valves, control valves. In some embodiments, control and sensing system 205 may include mini-fluidic and/or microfluidic elements. In some examples, the processors of the control and sensing system 205 may include microprocessors, microcontrollers, and the like.

The power source of the control and sensing system 205 may include external wired power systems, and/or batteries, and/or capacitors, and/or fuel cells, and/or wireless energy receiving devices as non-limiting examples. In some examples, the power source may be located outside of the control and sensing system 205 and provides power to the control and sensing system 205. As well, the sensors and control valves and check valves may be located throughout the apparatus 200A to support detection of IV infiltration. In some examples, the entire apparatus 200A to support detection of IV infiltration may be the boundary of the control and sensing system 205.

The apparatus 200A to support detection of IV infiltration may include an output connection 206. The output connection 206 may include standard locking elements such as luer fitting, a clave™ fitting, ridged connections, or other similar such sealing devices. In some examples, the output connection 206 may include a check valve or a control valve.

In some embodiments, the secondary input connection 203 may be replaced as a vessel of various kinds that may be integral with the apparatus 200A to support detection of IV infiltration. The vessel may initially contain a fluid such as saline or it may be filled after the apparatus 200A to support detection of IV infiltration is connected with an input fluid with the input fluid.

In some embodiments, the apparatus 200A to support detection of IV infiltration may include a pumping mechanism to pressurize fluid within such a vessel. In other examples, an included pump may allow for a temporary change in flow through the IV tubing to investigate the integrity of the IV installation.

FIG. 2B, item 200B, is a frontal view of the exemplary apparatus 200A to support detection of IV infiltration where the referenced elements are identical to those disclosed in the discussion related to FIG. 2A.

FIG. 2C, item 200C, is a frontal side top perspective view of the exemplary apparatus 200A to support detection of IV infiltration where the referenced elements are identical to those disclosed in the discussion related to FIG. 2A.

In some embodiments, an apparatus (200A) is configured to detect the state of IV infiltration and administer fluids or medications through an IV line installed in a patient. The apparatus comprises a comprehensive mechanism that integrates sensors, clamps, syringe holders, and communication capabilities to enhance the safety and efficiency of IV therapy.

The method begins by connecting the IV line to the apparatus. The primary input connection of the apparatus is linked to the IV line originating from a fluid source, such as a saline bag or medication reservoir. This connection establishes a pathway for fluid delivery. Simultaneously, the output connection of the apparatus is connected to the IV line leading to the patient's cannula. The apparatus serves as an intermediary that monitors and controls the fluid flow between the fluid source and the patient.

To facilitate medication administration, one or more syringe holders are placed on a secondary input connection integrated within the apparatus. These syringe holders are configured to accommodate syringes of varying sizes, providing compatibility with different therapeutic needs. For example, smaller syringes may be used for pediatric applications, while larger syringes are utilized for adult patients or higher-volume infusions. Each syringe holder is equipped with a spring mechanism that can be staged in a compressed or expanded state, allowing precise regulation of fluid injection. The springs are adjustable to vary the force applied during injection, providing compatibility with fluids of differing viscosities, such as saline, lipids, or medications.

A syringe containing a fluid solution, such as a secondary medication or hydration fluid, is placed within the syringe holder. The syringe is operably associated with the spring mechanism, which regulates the injection process. The spring's adjustable tension facilitates delivery of the fluid at the appropriate rate and pressure, minimizing the risk of tissue damage or infiltration.

The apparatus includes multiple sensors to monitor key parameters in the IV line. These sensors include a pressure sensor, which detects changes in pressure indicative of infiltration or blockages; a fluid flow sensor, which identifies irregularities in flow rate or direction; and a temperature sensor, which monitors fluid temperature to detect deviations from expected values. The sensors are operably connected to a processor integrated within the apparatus, enabling real-time data collection.

During operation, the apparatus releases an engagement arm on the syringe holder to activate the spring mechanism. This action pressurizes the fluid solution within the syringe, driving it into the IV line. The apparatus simultaneously monitors the pressure, flow rate, and temperature of the fluid in the IV line to detect any abnormalities. For example, if the pressure sensor detects an unexpected increase, this may indicate a blockage or infiltration.

The monitored parameters are recorded and stored in a data storage module within the apparatus. The storage module logs the data, including timestamps and injection details, for subsequent analysis. The processor uses this data to analyze deviations from normal conditions. For example, the processor may identify patterns of resistance in the IV line that correlate with infiltration or improper placement of the cannula. An artificial intelligence (AI) engine within the processor enhances this analysis by comparing real-time data to historical patterns and identifying trends indicative of potential issues.

The apparatus provides feedback to the user based on the processed data. Feedback can be delivered through a display integrated into the apparatus, visually presenting real-time information such as pressure trends, flow rates, and detected irregularities. In addition to visual feedback, the apparatus may be equipped with a communication module that transmits alerts and data to an external device, such as a smartphone or hospital monitoring system. For example, if IV infiltration is detected, the apparatus may send a notification to a nurse's smartphone, allowing immediate intervention.

The apparatus also incorporates clamps to regulate fluid flow in the IV line. A proximal clamp near the fluid source and a distal clamp near the patient's cannula are operably connected to the IV line. These clamps can selectively occlude the IV line to control fluid delivery. For example, during syringe injection, the proximal clamp may close while the distal clamp remains open, directing the medication toward the patient. Conversely, during syringe filling, the distal clamp may close to prevent backflow.

The design of the apparatus allows for seamless integration into various medical environments. The syringe holders and springs are adaptable for different syringe sizes and fluid types, providing versatility for diverse therapeutic applications. The sensors and AI-driven processing enable accurate monitoring and real-time decision-making, enhancing patient safety and improving clinical outcomes.

Referring now to FIGS. 2D-2F, these figures illustrate exemplary applications of the apparatus 200A connected to an IV line, showcasing configurations where the IV line is integrated into a medical setup. In such setups, the IV line serves as a conduit for delivering fluids from a fluid source 208 to a cannula 210 inserted into a vein of a patient 211. The apparatus 200A comprises several functional elements, including input tubing 201, activation element 202, secondary input connection 203, display means 204, control and sensing system 205, and output connection 206, all of which contribute to its operation in delivering and monitoring intravenous therapy. The integration of these elements with the IV line enhances the administration and monitoring of fluids and medications.

The IV setup depicted in FIG. 2D shows that the input tubing 201 of the apparatus 200A may be connected to an upper IV line 207A through a first Luer lock 212A. The upper IV line 207A is further connected to the fluid source 208, such as a saline bag, glucose solution, or medication pouch, containing a suitable fluid 209. These fluids are commonly used in medical procedures for hydration, electrolyte balance, or medication delivery. The use of a first Luer lock 212A facilitates a secure connection between the apparatus 200A and the IV line (207A-207B), preventing disconnection during operation.

The fluid source 208 may contain various types of fluids 209 depending on the patient's medical needs. For example, the fluid 209 may be a saline solution used for rehydration or as a vehicle for administering drugs. In other cases, the fluid 209 may be a glucose solution to manage blood sugar levels, an antibiotic solution for treating infections, or a chemotherapeutic agent for cancer treatment. Each type of fluid requires careful monitoring during administration, which is facilitated by the apparatus 200A.

The output connection 206 of the apparatus 200A is connected to a lower IV line 207B via a second Luer lock 212B, providing seamless transfer of fluids from the apparatus 200A to the lower IV line 207B leading to the patient 211. The lower IV line 207B terminates at a cannula 210, which is inserted into the vein of the patient 211. The cannula 210 serves as the final conduit for delivering fluids 209 into the patient's circulatory system. The placement of the cannula 210 requires precision to avoid complications such as infiltration or extravasation, and the apparatus 200A, with its integrated control and sensing system 205, assists in monitoring these parameters.

In one embodiment, the setup described in FIG. 2D may be used in emergency care to deliver fluids rapidly to a dehydrated patient. For example, the fluid source 208 may contain a saline solution 209, and the apparatus 200A can regulate the flow rate through the control and sensing system 205 to match the patient's requirements. The use of the input tubing 201, output connection 206, and integrated sensors facilitates accurate delivery while monitoring for anomalies in the flow.

Another embodiment involves the use of the apparatus 200A in chemotherapy. Here, the fluid source 208 may contain a chemotherapeutic agent 209 that needs to be administered at a controlled rate to avoid adverse effects. The input tubing 201 connects to the upper IV line 207A, allowing the chemotherapeutic agent (e.g., 209) to flow into the apparatus 200A. The control and sensing system 205 facilitates that the fluid 209 is delivered at the prescribed rate through the output connection 206 and lower IV line 207B to the patient's vein via the cannula 210. The display means 204 can provide real-time feedback on flow rate and pressure, allowing healthcare professionals to make required adjustments.

In yet another example, the apparatus 200A may be used in pediatric care, where the delivery of fluids requires extreme precision due to the smaller vein sizes in children. The fluid source 208 in such cases may contain a balanced electrolyte solution 209 to address dehydration. The input tubing 201 facilitates the entry of the fluid 209 into the apparatus 200A, where the control and sensing system 205 regulates the flow to avoid excessive pressure or flow rate, which may damage the veins. The output connection 206 provides the smooth transfer of fluids 209 to the lower IV line 207B and ultimately to the patient through the cannula 210.

The apparatus 200A also offers flexibility in accommodating different fluid sources 208 and IV line configurations. For example, the fluid source 208 may be a pressurized bottle containing a nutrient solution 209, and the input tubing 201 can handle the variable pressure. The control and sensing system 205 adjusts the flow to maintain consistent delivery, while the output connection 206 manages the transition to the lower IV line 207B. This adaptability makes the apparatus 200A suitable for diverse medical applications.

Furthermore, the integration of the control and sensing system 205 with the IV line setup allows for advanced monitoring capabilities. For example, pressure sensors within the system can detect blockages in the IV line, while flow sensors facilitates that the fluid 209 is moving at the prescribed rate. Acoustic sensors can detect anomalies such as air bubbles or infiltration. These features contribute to safer and more efficient intravenous therapy.

The patient 211 depicted in FIG. 2D represents a wide range of potential users of the apparatus 200A, including adults, children, and elderly individuals. Each of these groups presents unique challenges in intravenous therapy, such as varying vein sizes and tolerances to pressure. The apparatus 200A, with its customizable settings and advanced monitoring capabilities, addresses these challenges effectively. For example, in elderly patients with fragile veins, the apparatus 200A can be configured to deliver fluids 209 at a reduced pressure to minimize the risk of vein rupture.

The use of Luer locks 212A and 212B in the IV setup highlights the importance of secure connections in intravenous therapy. These connectors may be designed to prevent accidental disconnection during fluid administration, which may lead to interruptions in therapy or contamination of the IV line. The input tubing 201 and output connection 206 of the apparatus 200A are compatible with standard Luer lock fittings, facilitating a robust and reliable pathway for fluid transfer. The input tubing 201 directs the fluid 209 into the internal components (e.g., an internal reservoir or an internal fluid flow line) of the apparatus 200A for processing and monitoring.

Within the apparatus 200A, the fluid 209 may pass through an internal fluid reservoir or an internal fluid flow line (not depicted in FIG. 2D). This internal mechanism serves as a controlled pathway for the fluid 209, allowing for regulated flow and the integration of monitoring systems. The internal fluid reservoir or flow line may incorporate various sensors attached externally to its surface, forming part of the control and sensing system 205. These sensors are positioned to detect parameters related to the movement and characteristics of the fluid 209 without coming into direct contact with it. Such configuration maintains the sterility of the fluid pathway while providing real-time monitoring.

The output connection 206 provides an exit point for the fluid 209 from the apparatus 200A, facilitating its transfer to the lower IV line 207B. The secure attachment of the lower IV line 207B to the output connection 206 is achieved using a second Luer lock 212B, which prevents leaks or disconnections during operation. The lower IV line 207B directs the fluid 209 from the apparatus 200A to the cannula 210, which is inserted into the vein of the patient 211. The cannula 210 serves as the final interface for delivering the fluid 209 into the patient's circulatory system.

The control and sensing system 205 of the apparatus 200A may include sensors attached to the external surfaces of the internal fluid reservoir or flow line to detect various parameters associated with the fluid 209. These sensors include pressure sensors, flow sensors, acoustic sensors, and temperature sensors, among others. The placement of these sensors allows them to monitor the conditions of the fluid 209 indirectly by analyzing changes in the physical properties of the internal reservoir or flow line.

Pressure sensors may be used to measure changes in pressure within the internal fluid reservoir or flow line. These sensors can detect fluctuations in pressure caused by blockages, changes in flow rate, or resistance within the IV line. For example, if a blockage occurs downstream in the lower IV line 207B or the cannula 210, the pressure within the internal reservoir may increase. The pressure sensor, attached externally to the reservoir or flow line, detects these changes by analyzing the deformation or stress patterns on the surface of the reservoir or flow line material.

Flow sensors may monitor the movement of the fluid 209 through the internal reservoir or flow line by detecting vibrations or other physical changes on the external surface of the flow line. For example, an optical flow sensor positioned outside the flow line may emit light waves and measure the reflections caused by the movement of the fluid within. Variations in the reflected light can provide information about the speed and consistency of the flow. This allows for the detection of irregularities, such as reduced flow rates or sudden stoppages.

Acoustic sensors may be employed to monitor sound waves or sound wave patterns generated by the movement of the fluid 209 through the internal reservoir or flow line. These sensors are capable of identifying specific acoustic signatures associated with normal flow, air bubbles, or infiltration. For example, the presence of air bubbles in the fluid 209 may produce distinct sound patterns that can be detected by the acoustic sensor. The sensor, attached externally, picks up these sound waves through vibrations in the surface of the reservoir or flow line and transmits the data to the control system for analysis.

Temperature sensors may measure the temperature of the fluid 209 as it flows through the internal reservoir or flow line. These sensors can identify deviations from expected temperature ranges, which may indicate issues such as fluid exposure to external heat sources or delivery of fluids at incorrect temperatures. For example, a temperature sensor utilizing a thermistor may detect changes in heat conducted through the surface of the reservoir or flow line, providing valuable data without direct contact with the fluid.

In some embodiments, the apparatus 200A may integrate advanced sensing technologies such as infrared sensors or capacitive sensors to gather additional data about the fluid 209. Infrared sensors may analyze the thermal profile of the fluid 209 by measuring the heat radiated through the reservoir surface. Capacitive sensors may detect changes in the dielectric properties of the reservoir or flow line material caused by the presence or movement of the fluid 209.

The control system (e.g., processor and AI engine) of the apparatus 200A processes the data collected by these sensors to monitor and regulate the flow of the fluid 209. For example, during a saline infusion, the pressure sensor may detect a gradual increase in pressure within the reservoir, indicating a partial blockage in the lower IV line 207B. The control system can respond by adjusting the flow rate or alerting the medical professional through the display means 204 or by transmitting wireless alerts to a remote location. Similarly, during the administration of high-viscosity fluids, such as blood products or contrast agents, the flow sensor may detect changes in flow dynamics and optimize the delivery rate to maintain consistency.

The integration of these sensors allows the apparatus 200A to provide detailed insights into the status of the fluid 209 as it passes through the system, enabling healthcare professionals to make informed decisions about patient care. For example, during an antibiotic infusion, the acoustic sensor may detect air bubbles entering the internal reservoir from the upper IV line 207A. The control system can immediately pause the infusion and alert the operator, preventing potential complications.

In some embodiments of the present invention, the components of the apparatus 200A, including the input tubing 201, activation element 202, secondary input connection 203, display means 204, control and sensing system 205, output connection 206, and the internal reservoir or fluid flow line, may be manufactured from medical-grade materials. These materials are chosen to meet the stringent requirements of healthcare applications, offering properties such as biocompatibility, resistance to contamination, ease of sterilization, and durability. The use of medical-grade materials is intended to provide a reliable, safe, and hygienic environment for intravenous fluid administration.

The input tubing 201, constructed from medical-grade polymeric materials such as polyurethane or silicone, may offer flexibility, durability, and resistance to kinking. These properties allow the tubing 201 to maintain a consistent flow of fluid from the upper IV line 207A into the apparatus 200A. The medical-grade nature of the tubing facilitates that it is non-reactive with the fluids passing through it, such as saline solutions, glucose solutions, or medications. For example, in an embodiment where a chemotherapeutic agent flows through the input tubing 201, the material prevents leaching or degradation that may compromise the fluid's composition or safety.

The activation element 202, which facilitates controlled operations of the apparatus 200A, may also be composed of medical-grade components. For example, mechanical or electronic parts within the activation element may be encapsulated in medical-grade plastic or stainless steel, offering resistance to repeated usage and cleaning processes. In some embodiments, the activation element 202 may feature a touch-sensitive interface covered with a medical-grade antimicrobial coating to minimize the risk of pathogen transmission during operation.

The secondary input connection 203, designed for adding additional fluids or medications into the system, may be constructed from medical-grade materials such as polypropylene or polycarbonate. This component may feature a Luer lock connection to facilitates secure attachment to secondary IV lines or syringes. The medical-grade composition facilitates that the secondary input connection 203 remains sterile and resistant to fluid degradation. For example, when administering a secondary antibiotic infusion, the non-reactive nature of the connection prevents cross-contamination with the primary fluid.

The display means 204, used to provide real-time data on parameters such as flow rate, pressure, or infusion progress, may include a screen protected by a medical-grade acrylic or polycarbonate cover. This cover may be resistant to cleaning agents commonly used in medical environments, such as alcohol-based disinfectants, and offers high transparency for clear visibility. The medical-grade materials also prevent scratching or clouding, maintaining long-term usability in intensive care or outpatient settings.

The control and sensing system 205, encompassing electronic components and sensors, may be housed in a medical-grade casing to protect the internal mechanisms from environmental factors such as moisture, dust, or accidental spills. For example, a medical-grade polyether ether ketone (PEEK) enclosure may be used to house sensitive electronics while offering resistance to high-temperature sterilization processes. Sensors, such as pressure or flow sensors, may be coated with biocompatible materials that enable them to operate reliably when in contact with the internal fluid reservoir or flow line.

The output connection 206, which transfers the fluid from the apparatus to the lower IV line 207B, may be constructed from medical-grade materials designed for seamless integration with IV lines and cannulas. This connection may include a Luer lock mechanism made of medical-grade polypropylene, offering secure attachment to the lower IV line 207B while maintaining sterility. For example, during the administration of high-viscosity fluids, such as lipid-based nutrition solutions, the medical-grade construction prevents leaks or interruptions in the flow.

The internal fluid reservoir or flow line within the apparatus may also be made of medical-grade materials such as stainless steel or fluorinated ethylene propylene (FEP). These materials are selected for their non-reactive properties and resistance to microbial growth. The internal reservoir or flow line facilitates smooth and uninterrupted movement of fluid, and its medical-grade nature prevents contamination or degradation of the fluid during transit. For example, in an embodiment where the apparatus 200A delivers blood products, the non-stick properties of the flow line reduce the risk of clotting or residue buildup.

To enhance the hygiene and operational safety of the apparatus 200A, it may further comprise a UV sanitization setup integrated into the system. This setup may be designed to sanitize the input tubing 201, activation element 202, secondary input connection 203, display means 204, control and sensing system 205, output connection 206, and the internal fluid reservoir or flow line. UV sanitization offers an effective method for eliminating microorganisms, including bacteria, viruses, and fungi, without the use of chemicals or high temperatures.

The UV sanitization setup may include UV-C lamps strategically positioned within the apparatus to irradiate the surfaces of the aforementioned components. For example, the internal fluid reservoir or flow line may include a transparent section made of medical-grade quartz glass, allowing UV-C light to penetrate and sanitize the interior without interrupting fluid flow. The UV system may activate automatically during predefined intervals or when the apparatus is idle, maintaining a high level of hygiene without manual intervention.

In an embodiment, the UV sanitization setup may include multiple chambers dedicated to different components. For example, the input tubing 201 and output connection 206 may be placed in separate UV chambers, each equipped with reflectors to maximize light exposure. Sensors integrated into the UV system may monitor the intensity and duration of UV exposure, providing feedback to the control system to facilitate optimal sanitization.

The UV sanitization setup may also include a touchless activation mechanism to reduce the risk of contamination during operation. For example, a proximity sensor near the activation element 202 may detect when the apparatus 200A is not in use and automatically initiate a UV sanitization cycle. This feature may be useful in high-usage environments such as hospitals or emergency care units.

In applications involving immunocompromised patients, the integration of medical-grade materials and UV sanitization provides an additional safeguard against infection. For example, during the infusion of antibiotics for a patient with a weakened immune system, the sanitized components prevent the introduction of external pathogens, enhancing the reliability of the therapy.

In another embodiment, the UV sanitization setup may include a monitoring system that tracks the lifespan of the UV-C lamps and alerts the operator when replacement is required. This feature facilitates consistent performance of the sanitization system over time. Additionally, the apparatus 200A may include a display on the display means 204 to indicate the completion of each sanitization cycle, providing visual confirmation to the operator.

In some embodiments of the present invention, the sensors integrated within the apparatus 200A may be configured as intrusive sensors that are directly embedded into or in contact with the internal fluid reservoir or the internal fluid flow line. These sensors, being in contact with the fluid (209), are designed to monitor various parameters of the fluid passing through the apparatus and detect anomalies such as IV infiltration. By being in direct interaction with the fluid, these sensors can gather more precise and immediate data, enabling advanced monitoring and control capabilities for intravenous therapy.

The pressure sensors, as one type of intrusive sensor, may be embedded into the walls of the internal fluid flow line or reservoir, such that they directly interact with the fluid. These sensors can measure the absolute or relative pressure within the flow line, providing real-time feedback about the internal conditions of the fluid. For example, if infiltration occurs downstream of the apparatus, such as in the patient's vein near the cannula, the pressure within the flow line may fluctuate abnormally. The embedded pressure sensors detect these fluctuations by measuring the exerted pressure directly on their sensing surface. One embodiment of such a pressure sensor may utilize a piezoresistive element, which changes its electrical resistance based on the pressure applied. These changes are interpreted by the control system, allowing the apparatus to detect issues such as infiltration, occlusion, or irregular flow.

In another embodiment, flow sensors may be intrusive to the fluid reservoir or internal fluid flow line, providing direct measurement of the rate of fluid movement through the apparatus. These sensors may employ turbine or electromagnetic flow measurement technologies. For example, a turbine flow sensor may include a small rotor positioned within the flow line. As the fluid flows, the rotor spins at a speed proportional to the flow rate. By monitoring the rotor's rotations, the apparatus can calculate the exact flow rate. In cases of infiltration, the flow rate may decrease unexpectedly as the fluid leaks into surrounding tissues rather than continuing along the intended pathway. The flow sensor detects this anomaly and transmits the information to the control system, which may then pause the infusion and alert the operator.

Acoustic sensors, in contact with the fluid within the flow line or reservoir, may use ultrasonic or piezoelectric transducers to monitor sound waves generated by the movement of the fluid. For example, an ultrasonic acoustic sensor may emit sound waves through the fluid and analyze the returning signals. Changes in the acoustic properties, such as reflection patterns or transmission speed, may indicate the presence of air bubbles, infiltration, or blockages. For example, if air bubbles are introduced into the IV line, the acoustic sensor detects the difference in density and composition of the fluid, triggering a warning signal. This functionality is especially beneficial in preventing air embolisms during intravenous therapy.

Temperature sensors may also be intrusive, being positioned within the flow line or reservoir to directly measure the fluid temperature. These sensors may use thermocouples or thermistors that come into contact with the fluid to detect variations in temperature. Accurate temperature readings are important for administering certain fluids, such as blood products or contrast agents, which must be maintained within specific temperature ranges to preserve their efficacy and safety. For example, a thermocouple sensor embedded in the flow line may detect a rise in temperature caused by external heat exposure, allowing the control system to adjust parameters or alert the operator.

In some embodiments, conductivity sensors may be placed within the flow line or reservoir to measure the electrical conductivity of the fluid. These sensors utilize electrodes that come into direct contact with the fluid, measuring its ionic concentration. Changes in conductivity can indicate contamination, incorrect fluid composition, or dilution. For example, during electrolyte replacement therapy, the conductivity sensor can verify that the fluid contains the required balance of ions. If the conductivity deviates from expected values, the system may halt the infusion and prompt further investigation.

Another type of intrusive sensor that may be used in the apparatus is a chemical sensor, which directly interacts with the fluid to analyze its composition. These sensors may be particularly useful in detecting infiltration involving vesicant medications, such as chemotherapy agents, which can cause significant tissue damage if leaked into surrounding tissues. For example, a chemical sensor may use reactive coatings or optical methods to identify the presence of specific compounds within the fluid. If the sensor detects a deviation in medication concentration due to infiltration, it can alert the system to stop the infusion and notify the medical professional.

In an embodiment designed for advanced applications, optical sensors may be positioned within the fluid flow line to analyze the fluid's optical properties. These sensors may use lasers or light-emitting diodes to measure the fluid's turbidity, color, or refractive index. Changes in these properties can indicate anomalies such as blood contamination, medication crystallization, or air bubbles. For example, during the administration of antibiotics, the optical sensor can monitor the clarity of the solution. If the solution becomes turbid, indicating precipitation or contamination, the system can pause the infusion and notify the operator.

To enhance the functionality of the apparatus, the control system may be configured to process data from the intrusive sensors in real-time. This system may include advanced algorithms or artificial intelligence (AI) to analyze the sensor data and detect patterns indicative of infiltration or other complications. For example, the control system may use machine learning models trained on historical sensor data to differentiate between normal fluctuations and anomalies. When the system identifies a potential issue, it can automatically adjust the flow rate, activate alarms, or communicate with external devices to notify medical personnel.

In an embodiment designed for pediatric care, the apparatus 200A may include intrusive pressure and flow sensors calibrated for the lower pressure and flow rates required for small veins. These sensors provide precise measurements to avoid complications such as vein rupture or infiltration. The data collected by these sensors may be displayed on the apparatus's user interface or transmitted wirelessly to a medical professional's device for remote monitoring.

Another embodiment may integrate redundant intrusive sensors to increase reliability. For example, the apparatus 200A may include multiple pressure sensors placed at different points along the internal flow line. These sensors work together to identify inconsistencies in pressure readings, which may indicate infiltration or other issues. Redundant sensors enhance the robustness of the system, providing an additional layer of safety during intravenous therapy.

In scenarios involving high-viscosity fluids, such as blood or lipid-based nutrition solutions, the intrusive sensors can adapt to the unique properties of the fluid. For example, the flow sensor may include a specially designed rotor with low friction to accommodate the thicker consistency of such fluids. The acoustic sensor may also be optimized to differentiate between the sound signatures of high-viscosity fluids and normal saline.

Referring now to FIG. 2E, the apparatus 200A is connected directly to an upper IV line 207A and a lower IV line 207B without utilizing Luer locks (e.g., 212A-212B as described in FIG. 2D). This configuration demonstrates a streamlined setup, where the apparatus 200A facilitates uninterrupted fluid transfer from a fluid source 208 to a cannula 210 inserted into the vein of a patient 211. The direct connection between the apparatus 200A and the IV lines (207A-207B) eliminates the intermediary connectors, providing a simplified and compact system for intravenous fluid administration.

The upper IV line 207A, in such embodiments, extends from the fluid source 208 to the input tubing 201 of the apparatus 200A. The fluid source 208, which may be a saline bag, glucose pouch, or any other medical fluid container, delivers the fluid 209 directly through the upper IV line 207A to the apparatus. The input tubing 201 may be configured to securely attach to the upper IV line 207A using alternative connection mechanisms, such as threaded connectors or press-fit joints. These connections are designed to prevent fluid leaks and maintain a sterile pathway.

The input tubing 201 directs the fluid 209 into the internal mechanisms of the apparatus 200A, where it passes through an internal fluid reservoir or internal fluid flow line (not depicted in FIG. 2E). The input tubing 201, constructed from medical-grade materials such as silicone or polyurethane, offers flexibility and resistance to contamination, making it suitable for prolonged use in medical environments. The tubing may also include embedded ridges or grooves to enhance grip during manual connection to the upper IV line 207A.

Within the apparatus 200A, the fluid 209 may be regulated and monitored by the control and sensing system 205, which comprises various sensors to detect parameters such as pressure, flow rate, and fluid composition. These sensors, mounted externally or internally along the internal fluid flow line, interact with the fluid to gather useful data. For example, a flow sensor integrated into the control and sensing system 205 may use optical or electromagnetic techniques to measure the velocity of the fluid as it moves through the apparatus. This data can be displayed on the display means 204, providing real-time feedback to medical professionals.

The activation element 202, located on the body of the apparatus 200A, allows users to control various functions of the system. This element may be a physical button, a rotary knob, or a touch-sensitive interface that may provide intuitive control over fluid delivery parameters such as flow rate, injection timing, or pressure settings. In one embodiment, the activation element 202 may be designed with haptic feedback to indicate successful activation, enhancing user interaction in high-stress environments such as emergency rooms.

The secondary input connection 203 offers an additional pathway for introducing medications or secondary fluids into the system. This connection may include a press-fit or threaded port compatible with standard syringes or secondary IV lines. In practice, a healthcare provider may use the secondary input connection 203 to inject antibiotics or contrast agents into the system, which are then mixed with the primary fluid within the apparatus 200A before being delivered to the patient.

The output connection 206 transfers the fluid 209 from the apparatus 200A to the lower IV line 207B, which then delivers it to the cannula 210. The output connection 206, similar to the input tubing 201, may be constructed from medical-grade materials to maintain sterility and fluid integrity. This connection may feature integrated flow control valves or one-way check valves to regulate the direction and rate of fluid movement. The lower IV line 207B, extending from the output connection 206 to the cannula 210, serves as the final conduit for fluid delivery into the patient 211.

The cannula 210, inserted into the vein of the patient 211, completes the intravenous setup. This device, typically made of medical-grade stainless steel or polymer, is designed for compatibility with the lower IV line 207B and the human circulatory system. In one embodiment, the cannula 210 may include an integrated flow sensor to provide additional data on fluid delivery directly at the point of administration.

In an exemplary use case, the apparatus 200A depicted in FIG. 2E may be employed in a surgical setting where rapid fluid delivery is required. The absence of intermediary Luer locks in this configuration minimizes the risk of disconnections or leaks, allowing for uninterrupted operation. For example, during a surgery where blood volume restoration is required, the apparatus 200A can deliver saline or blood products directly from the fluid source 208 through the upper IV line 207A, the internal mechanisms of the apparatus, the lower IV line 207B, and the cannula 210.

Another embodiment of this configuration may involve the administration of high-viscosity fluids, such as lipid-based nutrition solutions or blood products. The direct connections between the upper IV line 207A, the apparatus 200A, and the lower IV line 207B reduce resistance and facilitate the smooth flow of these thicker fluids. The control and sensing system 205 can monitor parameters such as pressure and flow rate to optimize delivery.

In pediatric care, where vein sizes are smaller and more delicate, the apparatus 200A in the configuration shown in FIG. 2E may provide precise control over fluid delivery. The input tubing 201 and output connection 206, being constructed from flexible and biocompatible materials, reduce the risk of damage to the IV lines. The activation element 202 allows for quick adjustments to flow rate, so that the system adapts to the unique requirements of pediatric patients.

The apparatus 200A may also include additional safety features in this configuration. For example, the control and sensing system 205 may integrate acoustic sensors to detect air bubbles within the internal fluid flow line. If air is detected, the system can pause fluid delivery and notify the operator via the display means 204. This feature may be useful in preventing air embolisms during intravenous therapy.

The direct connection setup may also simplify maintenance and cleaning of the apparatus 200A. The absence of Luer locks reduces the number of components that need to be sanitized, and the use of medical-grade materials throughout the system facilitates repeated cleaning without degradation. In one embodiment, the apparatus may include a UV sanitization feature that automatically sterilizes the input tubing 201, output connection 206, and internal components between uses.

In a home-care setting, the configuration shown in FIG. 2E provides ease of use for non-professional caregivers. The direct connections between the IV lines and the apparatus 200A minimize setup complexity, while the intuitive activation element 202 and real-time feedback from the display means 204 allow for straightforward operation. For example, a caregiver administering hydration therapy to an elderly patient can use the apparatus to deliver fluids from the fluid source 208 with minimal training.

Referring now to FIG. 2F, the apparatus 200A may be connected to an uncut IV line 207 through a first Luer lock 212A positioned at a proximal point near the fluid source 208 and a second Luer lock 212B located at a distal point near the cannula 210. Unlike the configurations depicted in FIGS. 2D-2E, where the IV line 207 is divided into an upper IV line 207A and a lower IV line 207B, the setup in FIG. 2F maintains the continuity of the IV line 207. This configuration may allow some quantity of the fluid 209 from the fluid source 208 to bypass the apparatus 200A and flow directly to the cannula 210 through the segment of the IV line 207 located between the first Luer lock 212A and the second Luer lock 212B.

In some embodiments, the fluid 209 from the fluid source 208 flows through the IV line 207 towards the first Luer lock 212A, where a portion of the fluid 209 is diverted into the input tubing 201 of the apparatus 200A. The input tubing 201, securely attached to the IV line 207 via the first Luer lock 212A, channels the fluid 209 into the internal components of the apparatus 200A for monitoring, regulation, or modification. The remaining fluid 209 in the IV line 207 may continue unaltered toward the cannula 210 through the segment of the IV line 207 located between the first Luer lock 212A and the second Luer lock 212B.

Within the apparatus 200A, the diverted fluid 209 passes through an internal fluid reservoir or flow line (not explicitly shown in FIG. 2F) for controlled processing. The internal components of the apparatus 200A may include a control and sensing system 205, which comprises various sensors to detect pressure, flow rate, temperature, or composition of the fluid 209. These sensors, positioned along the external or internal surfaces of the reservoir or flow line, enable detailed analysis and regulation of the fluid before it exits through the output connection 206.

The output connection 206 may be attached to the IV line 207 at the second Luer lock 212B, allowing the processed fluid to re-enter the IV line 207 and flow toward the cannula 210. The second Luer lock 212B provides a secure reconnection of the fluid pathway, maintaining the sterility and integrity of the system. The fluid exiting the apparatus 200A merges with the portion of the fluid that bypassed the apparatus 200A, resulting in a combined flow delivered to the patient 211 through the cannula 210.

The configuration depicted in FIG. 2F offers flexibility in fluid administration by enabling a portion of the fluid 209 to bypass the apparatus 200A entirely. This can be particularly advantageous in scenarios where only a specific volume or composition of fluid requires modification or monitoring. For example, in a situation where the fluid source 208 contains a saline solution 209, the apparatus 200A may divert and process a portion of the fluid 209 for the addition of medication via the secondary input connection 203. The remaining saline solution in the IV line 207 between the first Luer lock 212A and the second Luer lock 212B flows directly to the cannula 210, providing uninterrupted hydration for the patient 211.

In another embodiment, the apparatus 200A may regulate the diverted fluid 209 to administer precise doses of medication or nutrients. The control and sensing system 205, equipped with sensors and actuators, monitors the flow and composition of the fluid within the apparatus 200A, adjusting parameters as needed. For example, during the administration of glucose for a diabetic patient, the apparatus 200A can divert a controlled portion of the fluid 209 for regulation and mixing with additional nutrients before reintroducing it into the IV line 207 through the second Luer lock 212B.

The use of the first Luer lock 212A and second Luer lock 212B in FIG. 2F provides a modular design that facilitates easy integration and detachment of the apparatus 200A from the IV line 207. This feature may be useful in dynamic healthcare environments, such as emergency care, where rapid changes to IV setups may be required. For example, if the apparatus 200A needs to be replaced or relocated, the Luer locks allow for quick disconnection and reconnection without compromising the sterility of the IV line 207.

The bypass segment of the IV line 207 between the first Luer lock 212A and the second Luer lock 212B also serves as a failsafe mechanism. In the event of a malfunction within the apparatus 200A, the fluid 209 in the bypass segment can continue to flow directly to the cannula 210, facilitating uninterrupted delivery to the patient 211. This feature may add a layer of reliability to the system, particularly in critical care settings where continuous fluid administration is paramount.

The apparatus 200A may further include advanced features to enhance its functionality in the configuration shown in FIG. 2F. For example, the display means 204 may provide real-time feedback on the volume of fluid 209 diverted into the apparatus 200A versus the volume bypassing it. This information can help healthcare providers monitor and adjust fluid delivery rates based on the patient's needs. Additionally, the activation element 202 may allow users to manually control the proportion of fluid 209 diverted into the apparatus 200A, offering greater flexibility in treatment plans.

In a pediatric care scenario, the configuration in FIG. 2F may be employed to administer precise doses of medication to a child while maintaining a consistent flow of hydration fluids. The apparatus 200A can divert a small volume of fluid 209 for mixing with medication and reintroduce it into the IV line 207 through the second Luer lock 212B, minimizing the risk of overdose while providing adequate hydration.

### Examples Of Apparatus To Support Detection Of IV Infiltration

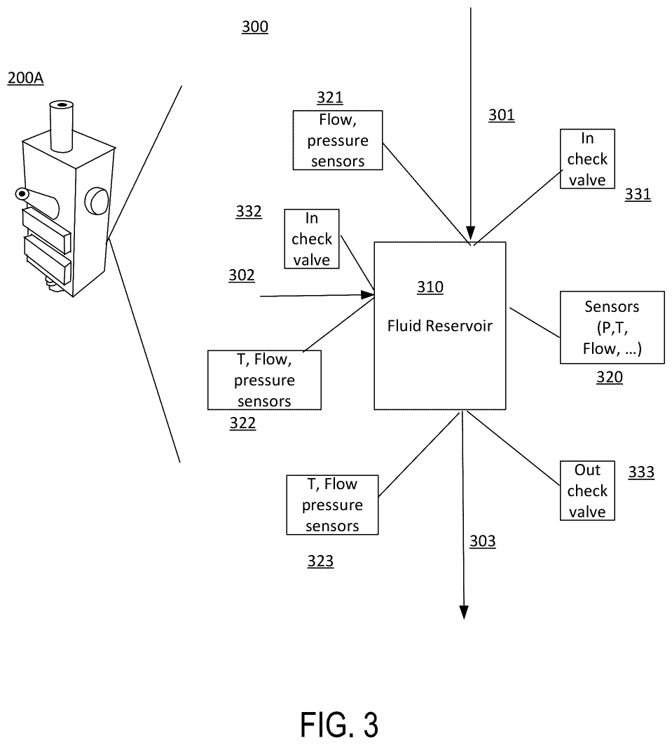

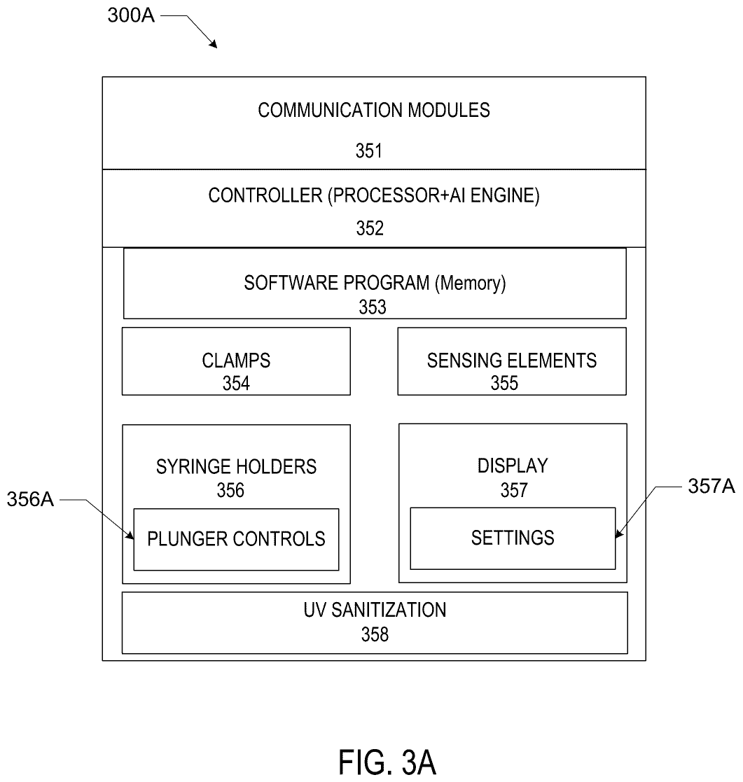

Referring to FIG. 3, functional elements that may be included in the apparatus (200A) to support detection of IV infiltration are illustrated in a block diagram 300 for a first example and for other examples.

The apparatus 200A to support detection of IV infiltration may include in its main body a fluid reservoir 310 which may also be considered a mixing chamber in some embodiments. The fluid reservoir 310 may include proximately located sensors 320 for measurements such as temperature, pressure, and flow as non-limiting examples of sensing capabilities. Temperature sensing may be performed by digital sensors such as resistor or diode-based sensors or other such digital sensors. Pressure measurement may be performed by digital sensors such as piezoelectric based sensors or other digital type pressure sensors. Flow sensors may include digital flow sensors using rotary motion encoding sensors, thermocouple or other temperature/heater-based sensors, or other digital sensors for flow sensing. These sensing capabilities may, in some embodiments, be performed with microfluidic/mini-fluidic solutions.

In some embodiments, the apparatus 200A to support detection of IV infiltration may include two or more input and output connections. In an example, the input connections may include a primary connection to input tubing 301, a secondary connection 302 and an output connection 303.

In some embodiments, the primary connection to input tubing 301 may include proximately located sensors 321 for measurements such as temperature, pressure, and flow through the primary connection to input tubing 301. In some embodiments, the primary connection to input tubing may include a check valve 331 or a controllable valve to regulate flow into the apparatus 200A to support detection of IV infiltration from the input tubing.

Likewise, the secondary connection 302 may include proximately located sensors 322 for measurements such as temperature, pressure, and flow through the secondary connection to input tubing 301. In some embodiments, the secondary connection 302 to input tubing 301 may include a check valve 332 or a controllable valve to regulate flow into the apparatus 200A to support detection of IV infiltration from the input tubing 301.

Moreover, the output connection 303 may include proximately located sensors 323 for measurements such as temperature, pressure, and flow through the output connection 303 to output tubing. In some embodiments, the primary connection to input tubing 301 may include a check valve 333 or a controllable valve to regulate flow into the apparatus 200A to support detection of IV infiltration from the input tubing.

Referring now to FIG. 3A, a block diagram illustrates exemplary internal components of the apparatus 300A (or 200A), which may be designed for IV infiltration detection and functioning as an infusion pump in some embodiments of the present disclosure. The apparatus 300A integrates multiple interconnected modules to provide advanced functionality, precision, and reliability in intravenous therapy management. Each component, represented within FIG. 3A, serves a distinct function while working synergistically with other components under the control of a central controller 352.

The communication modules 351, located at the top of the apparatus 300A in the block diagram, provide important connectivity features, including Wi-Fi, Bluetooth, and cellular SIM capabilities. These communication options allow the apparatus 300A to transmit real-time data and receive remote commands from medical professionals or caretakers. For example, the Wi-Fi module can connect the apparatus 300A to a hospital's local area network, enabling seamless integration with electronic medical records and centralized monitoring systems. Bluetooth connectivity provides a short-range option for pairing the apparatus 300A with handheld devices like tablets or smartphones, making it particularly useful in outpatient or homecare settings. The SIM-enabled cellular module allows the apparatus 300A to function in remote areas without Wi-Fi access, transmitting alerts or operational data via mobile networks. Each of these communication features facilitates that the apparatus 300A remains adaptable to a wide range of medical environments.

The controller 352 serves as the central processing unit of the apparatus 300A, orchestrating the operations of all connected components. It may include a processor for executing the software program 353 (software instructions) stored in a memory or data storage module and an artificial intelligence (AI) engine for advanced analytics. The processor manages routine tasks such as executing control commands for clamps 354, syringe holders 356, and plunger controls 356A. For example, when a medical professional sets specific infusion parameters, the processor executes these instructions, adjusting the apparatus 300A accordingly. The AI engine, on the other hand, enhances the precision and adaptability of the apparatus 300A by enabling features such as accurate sensing, predictive analytics, and machine learning. For example, the AI engine can analyze historical data from sensing elements 355 to predict potential IV infiltration risks before they occur, providing an added layer of safety for the patient.

The software program 353, executed by the processor within the controller 352, serves as the operational backbone of the apparatus 300A. This program includes algorithms for monitoring and controlling fluid flow, analyzing sensor data, and executing pre-set or user-defined commands. For example, the software program 353 may include routines for regulating the pressure applied by clamps 354 to the IV line or adjusting the speed and volume of fluid dispensed by the syringe holders 356. Additionally, the software program 353 may facilitate real-time communication with external devices via the communication modules 351, enabling remote operation and data sharing.

The clamps 354, controlled by the controller 352, may be designed to grip the IV line and regulate fluid flow at specific points. These clamps may include hydraulic or motorized mechanisms for dynamic adjustment of their grip strength. For example, during the administration of a secondary medication, the controller may instruct one clamp to tighten (close), temporarily blocking fluid flow from the primary fluid source, while the other clamp remains open to allow the medication to pass through to the patient. This precise control minimizes the risk of fluid backflow or over-infusion. The clamps 354 may also include integrated sensors to monitor parameters such as line pressure or flow rate, enabling them to detect and respond to potential anomalies.

The sensing elements 355, also managed by the controller 352, may provide important feedback regarding the status of the IV line and fluid flow. These sensors may include pressure sensors, acoustic sensors, and optical sensors, among others. For example, a pressure sensor within the sensing elements 355 can detect a sudden drop in line pressure, indicating possible infiltration or dislodgement of the IV catheter. Acoustic sensors, on the other hand, may analyze the sound of fluid moving through the IV line to identify irregularities such as air bubbles or occlusions. Optical sensors can be employed to monitor the clarity or composition of the fluid, detecting impurities or inconsistencies in medication delivery.

The syringe holders 356 may be designed to securely house one or more syringes, which are used for the administration of secondary medications or fluids. Each syringe holder may be equipped with a corresponding plunger control 356A, which may be motorized for automated operation. The controller 352 manages these plunger controls 356A to facilitate precise dosing based on user-defined settings or real-time feedback from the sensing elements 355. For example, during the administration of a controlled medication dose, the plunger control 356A depresses the syringe at a pre-set speed, delivering the exact volume of fluid into the IV line. This feature may particularly be useful in situations where high accuracy is required, such as during chemotherapy or pediatric care.

The display 357, located on the front panel of the apparatus 300A, provides a user-friendly interface for monitoring and adjusting settings. The display 357 may be a touchscreen or a conventional LCD, depending on the embodiment. The settings module 357A within the display 357 allows medical professionals to input parameters such as infusion rate, dose volume, and alarm thresholds. For example, during setup, the user can program the apparatus 300A to administer 5 mL of medication over a 10-minute period, with alerts triggered if the flow rate deviates by more than 10%. The display 357 also provides real-time feedback on system performance, showing data such as current pressure levels, flow rates, and remaining fluid volume.

The UV sanitization system 358, integrated within the apparatus 300A, may be configured to facilitate the sterilization of critical components such as the internal fluid flow line, syringe holders 356, syringes, clamps 354, and sensing elements 355. This feature may be advantageous in maintaining a hygienic environment, especially for reuse in clinical or home healthcare settings. The UV sanitization system 358 operates by emitting ultraviolet light, specifically within germicidal wavelengths (e.g., UV-C), to effectively eliminate or deactivate bacteria, viruses, and other pathogens on exposed surfaces. Controlled by the controller 352, the sanitization process can be initiated automatically before use, after each use or manually via settings on the display 357. This functionality provides optimal sterility without the need for manual disassembly and chemical cleaning, thus saving time and reducing the risk of contamination. Additionally, the apparatus 300A may log the sanitization cycles in the data storage module for compliance and maintenance tracking purposes.

The memory storage or data storage module (shown in 353) within the apparatus 300A serves multiple purposes, including the storage of operational logs, patient data, and software updates. For example, the apparatus 300A may use its memory to maintain a record of all infusion events, including timestamps, fluid volumes, and any alerts triggered during operation. This data can be invaluable for troubleshooting, compliance with medical regulations, and improving patient outcomes. The memory storage may also support machine learning by retaining historical data for analysis by the AI engine, enabling the apparatus to improve its performance over time.

In some embodiments, the communication modules 351 may enable the apparatus 300A to connect with cloud-based platforms for advanced data analytics. For example, the AI engine within the controller 352 can upload sensor data to a remote server, where more complex algorithms analyze the information and provide actionable insights to medical professionals. These insights may include predictions about IV line performance, recommendations for adjusting infusion parameters, or alerts about potential risks.

The modular architecture of the apparatus 300A allows for scalability and customization. For example, additional sensing elements 355 or syringe holders 356 can be integrated into the system to accommodate specific medical needs. In some embodiments, the apparatus 300A may include temperature sensors to monitor the thermal stability of the infused fluid, providing an additional layer of safety in scenarios where temperature-sensitive medications are administered.

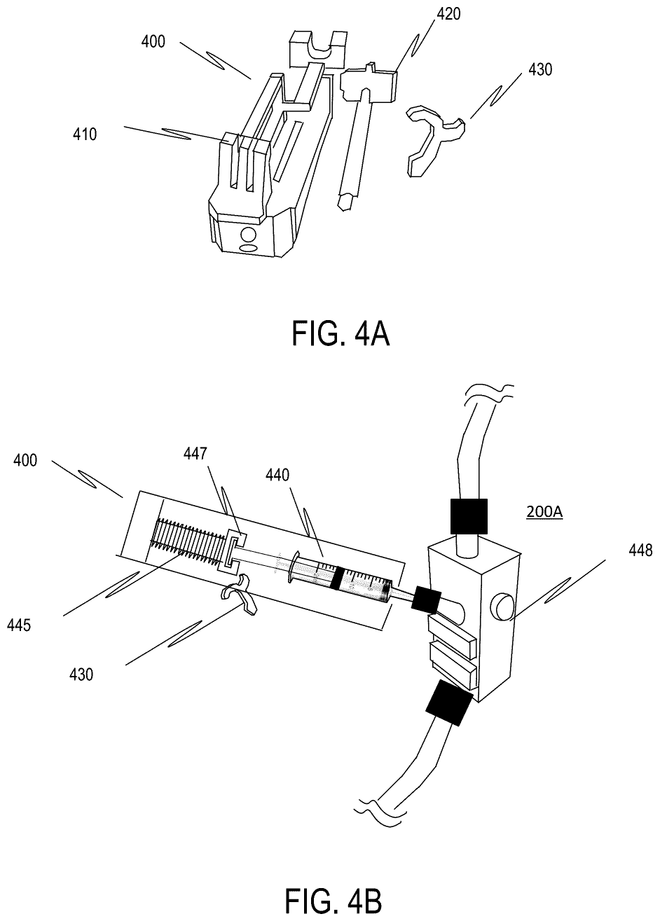

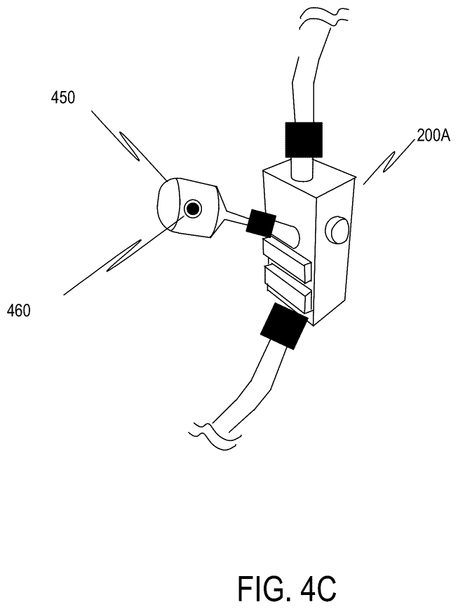

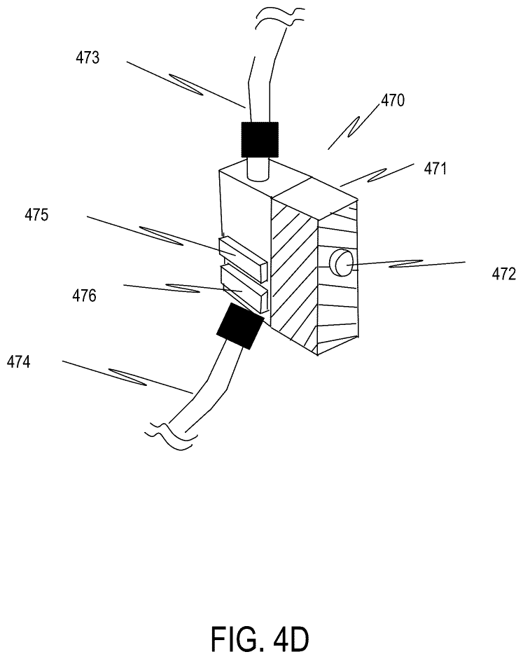

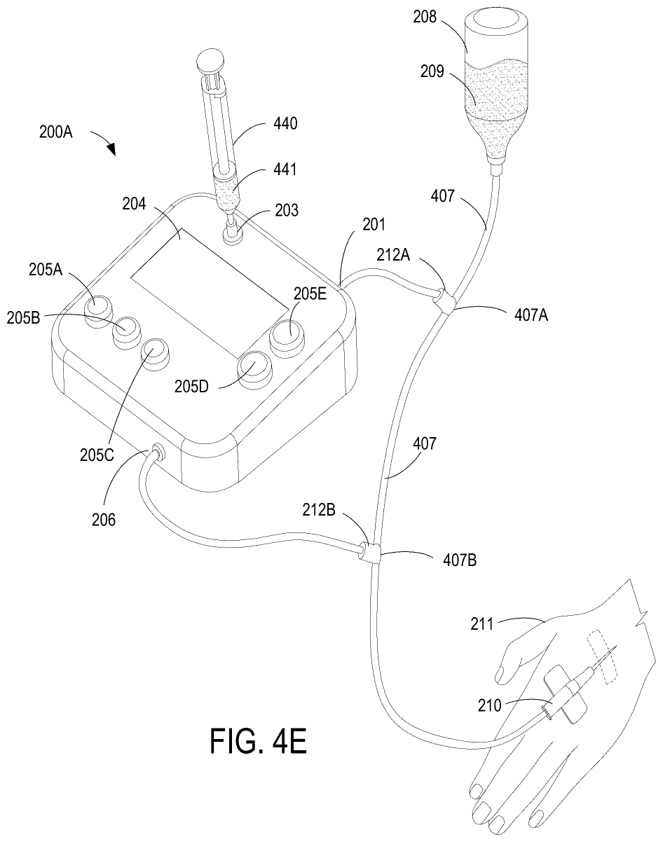

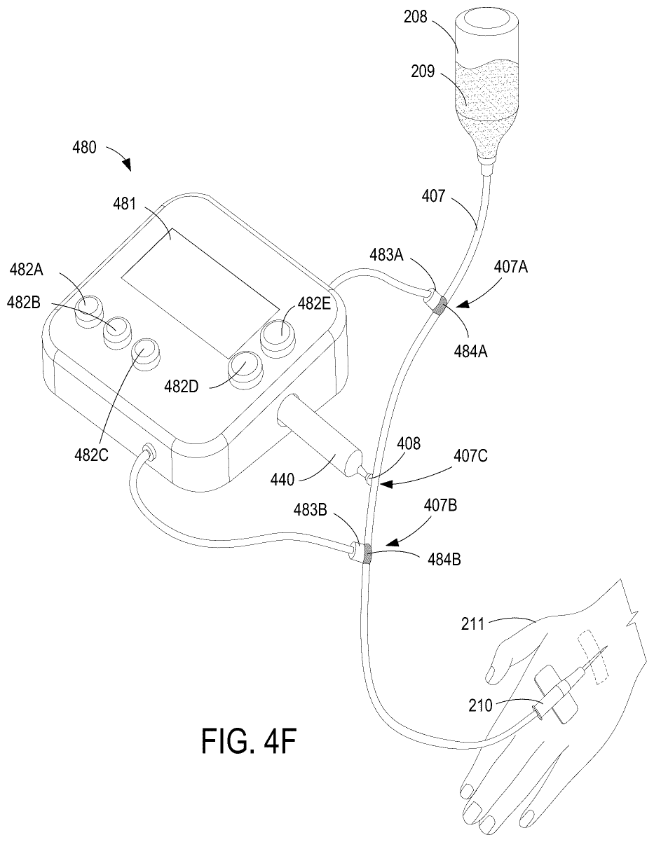

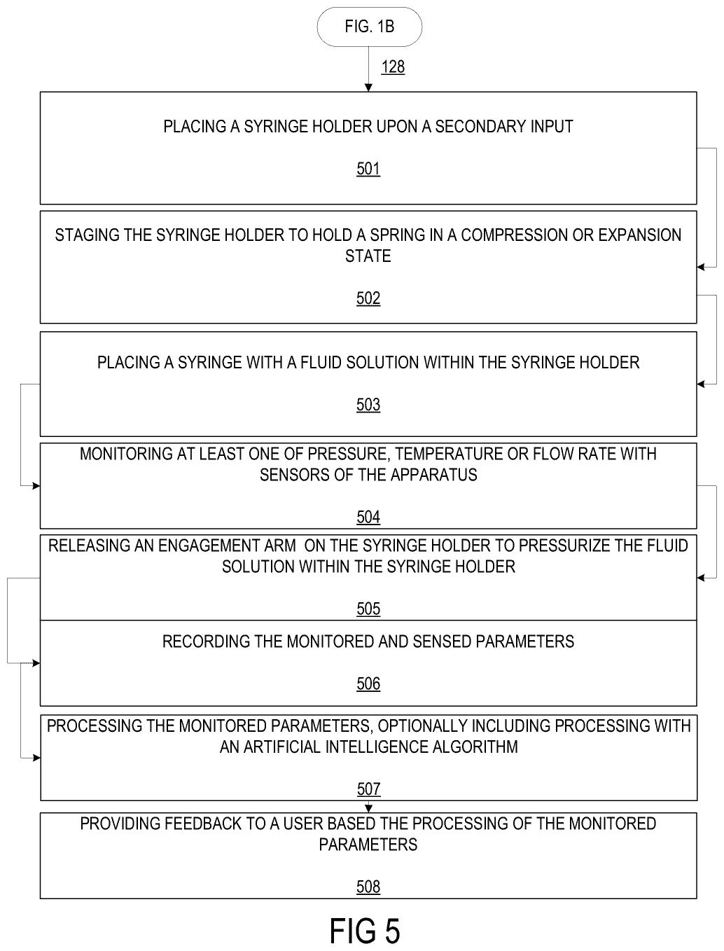

Referring now to FIG. 4A, a syringe controller may be used in concert with the apparatus 200A in the first example. In some embodiments, a syringe controller may utilize a releasable spring force to move a syringe member and flow syringe contents into the apparatus 200A to support detection of IV infiltration in a consistent and reproducible manner. A holding member 400 may include the structure 410 to support a syringe as well as a spring element that provides the force to move an engagement arm 420 which may provide the force on the plunger of the syringe. A release arm 430 may attach to the holding member 400 and hold the engagement arm 420 against the pressure of a spring against movement until its release.

Referring now to FIG. 4B, an illustration of the holding member 400 attached to the apparatus 200A to support detection of IV infiltration is provided. The apparatus 200A to support detection of IV infiltration may be prepared by compressing a spring 445 and locking an engagement arm 420 to hold the compressed spring and an interface 447 to connect a syringe to the holding member 400.

The apparatus 200A to support detection of IV infiltration may operate by placing a syringe 440 into the interface 447 and connecting to the secondary input connection. When the apparatus 200A to support detection of IV infiltration is readied to record sensed data, which may be initiated by engaging an activation device 448.

Next, a user may release the engagement arm 420 and the spring 445 may place a reproducible and predictable force vs time on the syringe arm causing the syringe to flow its fluid into the IV and the cannulation. A healthcare provider may trigger the apparatus 200A and may inject 10 ml of sterile crystalloid fluid into an existing IV under a specific pressure created by a stainless-steel spring member such as in a non-limiting example, spring LCM080F 05 S, from the Lee Spring Company which may have a free length inches 3.799 and generate a force of 1.073 pounds for each inch of compression. In some embodiments, well toleranced springs generate a reproducible force upon the syringe member.

In cases of a nominally operating IV, a pressurized fluid volume may flow into a vein with a relatively small amount of pressure buildup. Conversely, in cases of IV infiltration, the pressure that is measured may rise significantly, such as the pressure in an apparatus 200A to support detection of IV infiltration.

As opposed to a qualitative determination of a pressure rise by a medical professional, the measured curve of pressure vs time may be analyzed to trigger a communication to a user that an aberrant state is detected. The device may have a manometer, a flow sensor, timing electronics, and controllers that may be able to perform loops of flow/pressure, flow/time, and pressure/time loops. Resulting data may be compared with the historical results and expected loops for property working IVs and may be able to determine with an adequate degree of accuracy that the IV is infiltrated.

Data from many such assessments may be analyzed by experts and characterized. This data may be used as training data sets to train AI systems to result in even improved automatic detection of the aberrant states, especially as some aberrant states may not have strong signal levels.

Referring now to FIG. 4C, a pre-pressurized vessel that may be an alternative to the syringe and holder is illustrated. A pre-pressurized vessel 450 may include an activation element 460 to release the contents of the vessel and generate the pressure vs flow data with similar effect to the described relationship with the syringe-based injection of fluid into an IV. The pre-pressurized vessel may include a plunger and a cylindrical storage aspect and may even use a spring to generate the force. In other examples, compressed air or fluids may be used to generate the force. In an example, an elastic member may be stretched by pressurization of the IV test fluid into the vessel. In other examples an elastic membrane may contain another fluid or gas to provide the pressure to force the fluid out of the vessel. In some embodiments the activation element 460 may rupture a seal in a membrane. In other examples, the activation element 460 may have a mechanical holding function much as discussed in the prior example. In some embodiments, the activation element may be an electronic element such as a switch that may signal a controller or simple electrical circuit to activate a mechanical element. In some embodiments, an electrical element may flow an electrical current through a thin metal membrane to create a heat to rupture a membrane or other seal.

In another type of example, a vessel 450 may also be employed to perform the IV test protocol. In some embodiments of this type, the apparatus 200A to support detection of IV infiltration may comprise a pumping capability (for example, an infusion pump). The pumping capability may be used to directly pressurize IV fluid flow through the apparatus 200A. In another example, the pumping capability may be used to pump normal IV fluid content into the vessel 450 to be released under pressure to perform the analysis for a nominal IV function as previously described.

Referring now to FIG. 4D, in some embodiments, a vessel 471 may be included as part of the apparatus 200A to support detection of IV infiltration 470. As illustrated, an activation element 472 may be used to initiate a process in the controller 475 of the device. The controller 475 may actuate valves to divert IV fluid from an input connection 473 through a pump within the device to store the IV fluid in the vessel 471. At a later time, in some embodiments, the controller may close a valve in the input connection 473 and then actuate the pump of the device to pressurize fluid to flow into the output connection 474. Sensor devices 476 within the device may characterize the pressure vs time and flow aspects as have been mentioned to characterize the IV integrity and support detection of IV infiltration. An output may be displayed on a display function 481 which may include the various types of display as have been previously described. In some embodiments, the device may repeat the steps of filling the reservoir and pumping IV fluid to ascertain IV integrity over the full use cycle of the IV cannulation.

Referring now to FIG. 4E, a compact embodiment of the apparatus 200A is illustrated, the apparatus 200A is equipped with a syringe 440 configured to introduce a secondary medicine or fluid 441 into the IV system. The apparatus 200A may be connected to the IV line 407, which originates from a fluid source 208 and terminates at a cannula 210 inserted into the patient 211. The connection is facilitate through a first Luer lock 212A located at a proximal point 407A near the fluid source 208 and a second Luer lock 212B at a distal point 407B near the cannula 210. This setup maintains the continuity of the IV line 407 while enabling the apparatus 200A to regulate and monitor the fluid 209 and deliver the secondary medicine 441 from the syringe 440.

The syringe 440, positioned on the apparatus 200A, may be used to introduce secondary medicine or fluid 441 into the IV system through the secondary input connection 203. The syringe 440 may be manually operated or integrated with an infusion pump that automates the delivery process. In one embodiment, the infusion pump may be built into the apparatus 200A, allowing seamless integration and operation. For example, the infusion pump may use a motorized plunger mechanism to dispense the secondary medicine 441 at a controlled rate. The motorized mechanism may be connected to the control and sensing system 205, which regulates the timing, volume, and pressure of the delivery based on pre-set parameters.

In another embodiment, the syringe 440 and its infusion pump may be externally attached to the apparatus 200A. This configuration provides modularity, allowing healthcare professionals to connect different types of infusion pumps depending on the required application. For example, a volumetric pump may be used for precise dosing of high-viscosity medications, while a simpler spring-loaded pump may suffice for standard fluid delivery. The secondary input connection 203 facilitates the integration of the syringe 440 into the fluid pathway of the apparatus 200A, facilitating a seamless blend of the secondary medicine 441 with the primary fluid 209 from the fluid source 208.

The apparatus 200A may further be equipped with a series of control and setting buttons 205A-205E, each dedicated to a specific function. The button 205A may be configured to set the timing for the automatic activation of the syringe 440. For example, a healthcare provider may program the apparatus 200A to deliver a dose of the secondary medicine 441 every hour, facilitating consistent and accurate administration. Button 205B may allow the operator to set the quantity of the secondary medicine 441 to be dispensed. For example, the apparatus 200A can be adjusted to deliver doses ranging from 0.1 mL to 10 mL, depending on the patient's treatment plan.

Button 205C may be used to regulate the flow rate of the primary fluid 209 from the fluid source 208. For example, in hydration therapy, the operator can use this button to adjust the flow rate to 20 drops per minute. Button 205D may provide a manual override function, allowing the operator to pause or resume the infusion process as needed. This feature may particularly be useful in situations where immediate adjustments are required, such as during the administration of emergency medications. Button 205E may enable the operator to initiate a priming sequence, where the apparatus flushes the IV line 407 with the fluid 209 or secondary medicine 441 to eliminate air bubbles before delivery to the patient 211.

The display means 204 works in conjunction with the control and setting buttons 205A- 205E to provide visual feedback and confirmation of the selected parameters. For example, when the operator adjusts the flow rate using button 205C, the display means 204 shows the updated rate in real time, allowing the operator to verify the settings. Similarly, during the programming of dose intervals using button 205A, the display means 204 may provide a countdown timer indicating when the next dose will be delivered. This interaction between the control buttons 205A-205E and the display means 204 enhances the usability and accuracy of the apparatus 200A.

The compact design of the apparatus 200A depicted in FIG. 4E makes it suitable for various healthcare settings, including outpatient clinics, emergency rooms, and home care environments. For example, in a home care setting, a caregiver can use the apparatus 200A to administer a pre-mixed antibiotic solution through the syringe 440 while maintaining a continuous flow of saline from the fluid source 208. The control buttons 205A-205E and display means 204 simplify operation, reducing the need for extensive training or supervision.

In another embodiment, the apparatus 200A may incorporate advanced connectivity features to enhance its functionality. For example, the apparatus 200A may include wireless communication modules that allow the control buttons 205A-205E to be programmed remotely through a mobile application. This feature enables medical professionals to adjust the settings of the apparatus 200A without being physically present, offering greater flexibility and convenience.

The integration of the syringe 440 with the apparatus 200A also opens up possibilities for personalized treatment plans. For example, in oncology settings, the apparatus 200A can be programmed to deliver chemotherapeutic agents through the syringe 440 at specific intervals while simultaneously administering hydration fluids through the IV line 407. The precise control offered by the control and sensing system 205 facilitates that the treatment is tailored to the patient's individual needs.

The use of medical-grade materials in the construction of the syringe 440, secondary input connection 203, and other components provides compatibility with a wide range of fluids and medications. For example, the syringe may be made from medical-grade polypropylene, offering resistance to chemical degradation when exposed to aggressive drugs such as cytotoxic agents. The secondary input connection 203 may include a silicone gasket to prevent leaks during the integration of the syringe with the apparatus 200A.

Referring now to FIG. 4F, an exemplary apparatus 480 serves as a combination of an infusion pump and an IV infiltration detection device. The apparatus 480 is depicted in an assembled configuration connected to an IV line 407 and features a range of advanced functionalities for administering fluids and detecting potential anomalies during intravenous therapy. The apparatus 480 is compactly designed and comprises a display screen 481, a series of control buttons 482A-482E, and a syringe 440, which may be housed within a syringe holder inside the apparatus 480. Additionally, the apparatus 480 interacts with the IV line 407 at specific points using a first clamp 483A at a proximal point 407A and a second clamp 483B at a distal point 407B. The syringe 440 may be connected to the IV line 407 at an intermediate point 407C through a Luer lock 408.

The display screen 481, prominently located on the surface of the apparatus 480, provides real-time information regarding the operation of the apparatus 480. This screen may display important data, including flow rate, pressure readings, and the status of fluid administration or alerts for IV infiltration. The graphical interface of the display screen 481 may further enhance usability by showing dynamic parameters such as the volume of fluid delivered through the syringe 440 or the pressure exerted by the clamps 483A and 483B on the IV line 407. For example, during a therapeutic infusion, the medical professional can monitor the progress of fluid delivery while receiving instant feedback about any detected anomalies.

The apparatus 480 may include a plurality of control buttons 482A-482E, each assigned to a specific function to regulate and operate the infusion and detection systems. Button 482A may be programmed to initiate the infusion process, starting the motorized action of the syringe 440 to dispense medication or fluids into the IV line 407 through the Luer lock 408. Button 482B may allow the user to adjust the flow rate of the fluid, enabling precise control over the speed at which the secondary medicine is administered to the patient 211. For example, in a scenario where small and controlled doses of medication are required, button 482B may set the flow rate to as low as 0.1 mL per second.

Button 482C may serve as a calibration button for the sensing elements 484A and 484B integrated into the first clamp 483A and the second clamp 483B, respectively. Calibration facilitates that the sensors are tuned to detect changes accurately in parameters such as flow resistance, pressure, or acoustic signals within the IV line 407. Button 482D may provide an override function to manually deactivate the clamps 483A and 483B in case of an emergency or during maintenance. Lastly, button 482E may activate advanced features such as data logging or wireless connectivity for remote monitoring through external devices like smartphones or hospital information systems.

The syringe 440, positioned within the apparatus 480, may be configured to introduce secondary medicine or fluids into the IV line 407 at point 407C via the Luer lock 408. The syringe 440 may be operated using a motorized infusion pump housed within the apparatus 480, enabling automatic and controlled delivery of the secondary fluid. For example, during the administration of pain relief medication alongside saline from the fluid source 208, the syringe 440 can dispense pre-set doses at specific intervals. The motorized mechanism facilitates precise administration, reducing the risk of underdosing or overdosing.

The first clamp 483A and the second clamp 483B are positioned to encircle the IV line 407 at the proximal point 407A and the distal point 407B, respectively. These clamps may be equipped with sensing elements 484A and 484B, which are integral to the infiltration detection capabilities of the apparatus 480. The sensing elements 484A and 484B are designed to monitor parameters such as pressure, flow resistance, and acoustic signals within the IV line 407. For example, the sensing element 484A may include a piezoelectric pressure sensor that detects changes in the internal pressure of the IV line 407 at the proximal point 407A. If infiltration occurs downstream, the pressure may drop abnormally, triggering an alert through the display screen 481.

The sensing element 484B, integrated into the second clamp 483B at the distal point 407B, may utilize acoustic sensors to monitor the sound of fluid flow within the IV line 407. These acoustic sensors may detect irregularities such as air bubbles, occlusions, or leakage into surrounding tissues. For example, if the acoustic signature deviates from the baseline due to infiltration, the sensing element 484B transmits a signal to the control system of the apparatus 480, which may display an alert on the display screen 481 and pause the infusion.

In some embodiments, the sensing elements 484A and 484B may work in tandem to compare pressure and flow rate data from the proximal and distal points of the IV line 407. Any significant discrepancies between the readings can indicate issues such as catheter dislodgement or partial occlusion. For example, if the flow rate detected at the proximal point 407A is significantly higher than that at the distal point 407B, the system may infer the presence of leakage or infiltration and alert the operator.

The clamps 483A and 483B may also include adjustable mechanisms to apply varying degrees of pressure on the IV line 407. For example, during the detection of infiltration, the clamps 483A-483B can momentarily restrict fluid flow to isolate the affected segment of the IV line 407 for further analysis. This functionality allows the apparatus 480 to dynamically adapt to changing conditions during intravenous therapy.

The Luer lock 408, connecting the syringe 440 to the IV line 407 at point 407C, facilitates a secure and leak-proof interface. This connection allows the secondary medicine or fluid introduced by the syringe 440 to seamlessly integrate with the primary fluid 209 from the fluid source 208. The apparatus 480 may monitor the flow and composition of the combined fluids using the sensing elements 484A and 484B, providing consistent and safe delivery to the patient 211.

The apparatus 480, as shown in FIG. 4F, represents a versatile solution for intravenous therapy, combining infusion and infiltration detection functionalities in a single compact unit. The integration of advanced sensors, precise control mechanisms, and real-time feedback systems makes it suitable for diverse medical applications, including emergency care, outpatient therapy, and home care settings. The detailed embodiment provided herein demonstrates the adaptability and reliability of the apparatus 480 in addressing the complexities of intravenous fluid administration and monitoring.

In some embodiments, the first clamp 483A may be configured to grip the IV line 407 at the proximal point 407A, allowing precise control of the flow of fluid 209 from the fluid source 208. The first clamp 483A may use an adjustable gripping mechanism that tightens or loosens around the IV line 407 based on the flow requirements. This adjustment may be controlled by a hydraulic system or hydraulic actuator connected to the first clamp 483A. The hydraulic system may include a hydraulic line that supplies pressure to the first clamp 483A, and this pressure is regulated by a controller housed within the apparatus 480. The ability to modulate the grip of the first clamp 483A allows for controlled dripping or flow of the fluid 209 into the IV line 407, making it highly adaptable to different medical scenarios.

The gripping mechanism of the first clamp 483A may be designed to encircle the IV line 407 uniformly to prevent deformation or damage to the tubing. For example, the inner surface of the clamp 483A may be lined with a soft, flexible material such as silicone, which conforms to the shape of the IV line 407 while maintaining a firm grip. The hydraulic line connected to the first clamp 483A exerts variable pressure, which the controller adjusts based on the desired flow rate. For example, if a slower drip rate is required, the controller increases the hydraulic pressure to tighten the grip of the first clamp 483A, partially restricting the flow of fluid 209.

In situations where a rapid flow of fluid 209 is needed, such as in emergency hydration therapy, the controller reduces the hydraulic pressure in the hydraulic line, loosening the grip of the first clamp 483A. This allows a larger volume of fluid 209 to pass through the IV line 407 at a faster rate. The modulation of hydraulic pressure may be achieved through a motorized pump or a solenoid valve integrated within the apparatus 480, which responds to commands from the controller. The adjustments can be programmed in advance or made dynamically in response to real-time feedback from the sensing elements 484A and 484B.

The second clamp 483B, positioned at the distal point 407B of the IV line 407, may operate similarly to the first clamp 483A and provides additional control over the fluid pathway. The controller within the apparatus 480 coordinates the actions of both clamps to manage the flow of fluid 209 effectively. For example, during the administration of a secondary medicine from the syringe 440, the controller may close the first clamp 483A to block the flow of fluid 209 from the fluid source 208, while keeping the second clamp 483B open. This creates a unidirectional flow of the secondary medicine from the syringe 440 through the IV line 407 to the cannula 210.

The integration of the clamps 483A and 483B with the controller allows for complex operational sequences that enhance the functionality of the apparatus 480. For example, during an infusion cycle, the controller may alternate between opening and closing the clamps 483A-483B to perform flushing operations. In one embodiment, the controller briefly closes the second clamp 483B and opens the first clamp 483A to allow a small volume of fluid 209 to flush the syringe connection point 407C, preventing clogging or sediment buildup.

The controller may also utilize data from the sensing elements 484A and 484B to adjust the clamps 483A-483B dynamically. For example, if the sensing element 484A detects an abnormal pressure drop at the proximal point 407A, the controller may tighten the grip of the first clamp 483A to stabilize the flow. Similarly, if the sensing element 484B identifies irregular acoustic signals (acoustic resistance) indicative of infiltration at the distal point 407B, the controller may temporarily close the second clamp 483B to isolate the affected segment of the IV line 407 for further analysis.

The infusion pump for the syringe 440 may be another useful component controlled by the controller. When it is time to administer the secondary medicine, the controller activates the motorized mechanism of the infusion pump to depress the plunger of the syringe 440, delivering the medicine into the IV line 407 at point 407C. The controller coordinates this action with the clamps, closing the first clamp 483A to prevent backflow into the fluid source 208 while keeping the second clamp 483B open to allow the medicine to flow toward the cannula 210 and into the patient 211.

In one embodiment, the controller can execute pre-programmed infusion protocols tailored to specific medical treatments. For example, during chemotherapy, the controller may administer the secondary medicine from the syringe 440 in small, controlled doses over a set period. The clamps 483A and 483B adjust their grip on the IV line 407 accordingly to maintain a consistent flow rate and prevent interruptions.

The hydraulic lines connected to the clamps 483A and 483B may be equipped with pressure sensors to monitor the force applied to the IV line 407. These sensors provide feedback to the controller, so that the clamps 483A-483B exert the optimal amount of pressure without damaging the tubing. For example, during pediatric care, where the IV lines are more delicate, the pressure sensors help the controller apply a gentler grip, reducing the risk of line deformation or leakage.

In addition to hydraulic control, the clamps 483A and 483B may feature mechanical backup systems for manual operation. In case of a power failure or system malfunction, a medical professional can manually tighten or loosen the clamps 483A-483B using adjustment screws or levers. This redundancy enhances the reliability of the apparatus 480, making it suitable for critical care settings.

The modular design of the clamps 483A and 483B allows them to be easily detached and replaced, facilitating maintenance and sterilization. For example, after a surgical procedure, the clamps 483A-483B can be removed from the apparatus 480 and cleaned separately using standard medical-grade disinfectants. This feature may particularly be useful in environments where multiple patients are treated using the same apparatus.

### Methods Of Use Of An Apparatus To Support Detection Of IV Infiltration

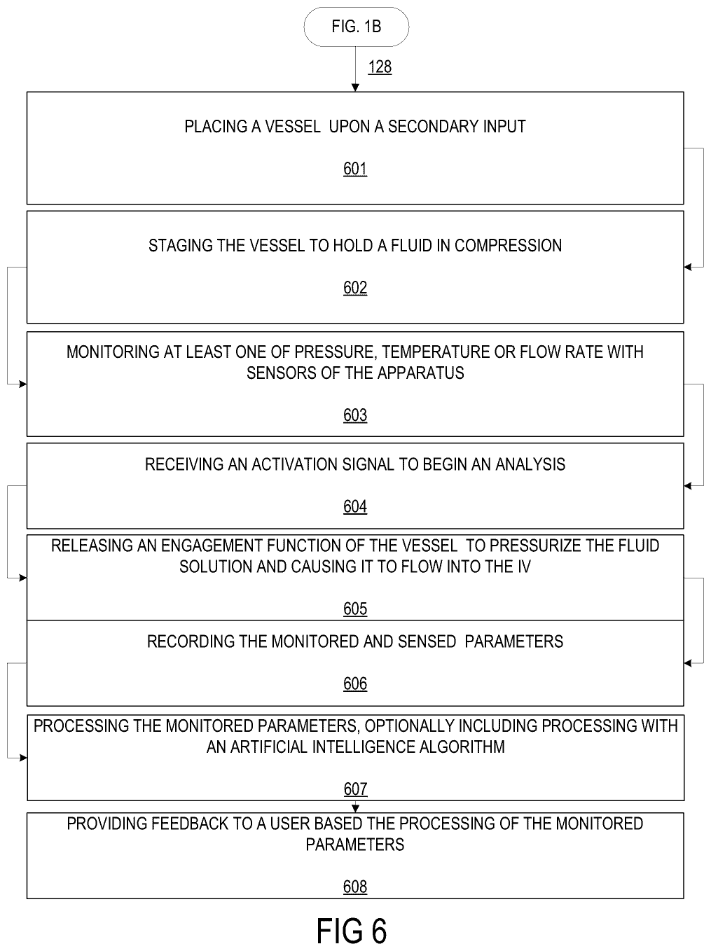

Referring now to FIG. 5, additional method steps 128 may be performed in addition to some, or all of the steps illustrated and described in reference to FIGS. 1A-1B. In some embodiments, the additional method steps may include step 501, placing a syringe holder upon a secondary input. As has been described the syringe holder may be attached to a secondary input port or other support structure on the apparatus to support detection of IV infiltration. In some additional examples, the method may include step 502, staging the syringe holder to hold a spring in a compression or expansion state. The staging may involve a manual movement of holder components to compress the spring. In some embodiments, the spring may be connected so that an expansion of the spring is staged.

In some embodiments, the methods may include step 503, placing a syringe with a fluid solution within the syringe holder. In some embodiments, the methods may include step 504, monitoring at least one of pressure, temperature, or flow rate with sensors of the apparatus. The storage may be initiated by interaction with an activation element of the device. In some embodiments, the method may include a step 505, releasing an engagement arm on the syringe holder to pressurize the fluid solution within the syringe holder. In some embodiments, the method may include step 506, recording the sensed and monitored parameters. In some embodiments, the method may include step 507, processing the monitored parameters, optionally including processing with an artificial intelligence algorithm. And, furthermore, the method may include step 508, providing feedback to a user based on the processing of the monitored parameters.

Referring now to FIG. 6, additional method steps 128 may be performed in addition to some, or all of the steps illustrated and described in reference to FIGS. 1A-1B. In some embodiments, the additional method steps may include step 601, placing a vessel upon a secondary input. As has been described the vessel may be attached to a secondary input port or other support structure on the apparatus to support detection of IV infiltration. In some embodiments, the vessel may be an integral part of the device. In some additional examples, the method may include step 602, staging the vessel to hold a fluid in compression. The staging may involve a manual movement of holder components. In some embodiments, the methods may include step 603, monitoring at least one of pressure, temperature, or flow rate with sensors of the apparatus. In some embodiments, the methods may include step 604, receiving an activation signal to begin an analysis. In some embodiments, the method may include a step 605, releasing an engagement function of the vessel to pressurize the fluid solution and causing it to flow into the IV. In some embodiments, the method may include step 606, recording the sensed and monitored parameters. In some embodiments, the method may include step 607, processing the monitored parameters, optionally including processing with an artificial intelligence algorithm. And, furthermore, the method may include step 608, providing feedback to a user based on the processing of the monitored parameters.

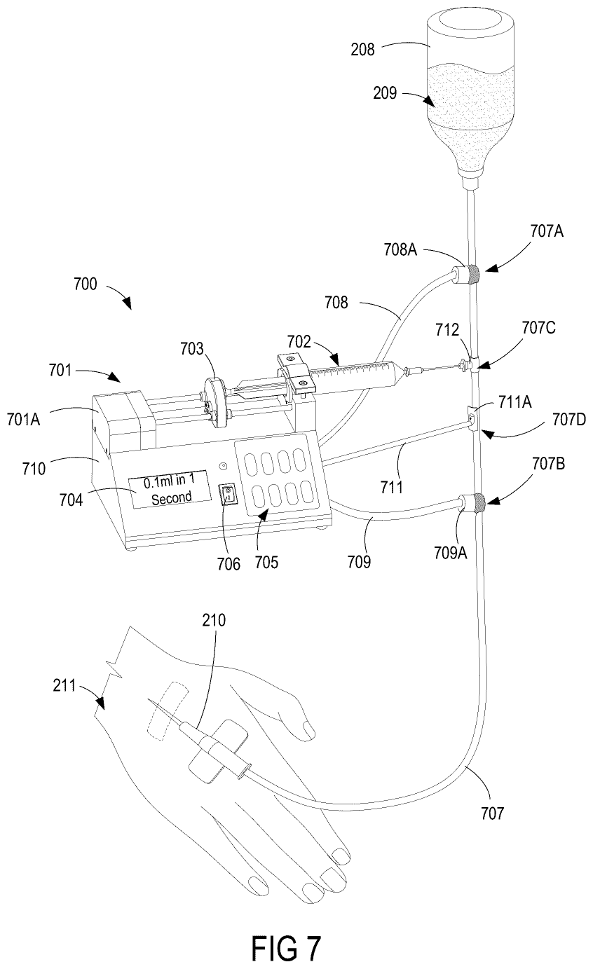

Referring now to FIG. 7, an apparatus 700 integrated with an infusion pump and IV infiltration detection system is connected to an IV line 707 for administering fluids and monitoring flow conditions during intravenous therapy. The apparatus 700 is shown to comprise multiple functional components, including a syringe holder 701, a syringe 702 containing a secondary medicine or fluid, a motorized plunger 703 controlled by a plunger control 701A, a housing 710 encapsulating the internal components of the apparatus 700, a display screen 704 for monitoring and controlling the apparatus 700, control buttons 705 for configuring operational parameters, and a power on/off switch 706 for activating or deactivating the apparatus 700.

The syringe holder 701 is designed to securely hold the syringe 702 in place during operation. The syringe 702 may be configured to store a secondary medicine or fluid, which is injected into the IV line 707 through a first connection point 707C using a Luer lock 712. The plunger 703 of the syringe 702 may be motor-operated and controlled by the plunger control 701A. This motorized mechanism allows for precise compression and expansion of the plunger 703, enabling controlled dispensing of the secondary fluid into the IV line 707. For example, during the administration of a specific dose, the plunger control 701A may depress the plunger 703 at a predetermined rate to provide accurate and uniform fluid delivery.

The housing 710 encloses various components of the apparatus 700, including the motorized plunger control 701A, hydraulic systems or hydraulic actuators, and electronic circuits required for device operation. The housing 710 may be constructed from durable, medical-grade materials that are resistant to corrosion and compatible with hospital sterilization protocols. This robust design allows the apparatus 700 to operate reliably in diverse healthcare settings, including outpatient clinics, intensive care units, and home care environments.

The display screen 704 may be positioned on the front of the housing 710 and provides a user-friendly interface for monitoring and controlling the device. The display screen 704 may display real-time information, such as the flow rate of the fluid 209, the pressure levels within the IV line 707, and the volume of secondary medicine delivered by the syringe 702. This feedback enables healthcare professionals to monitor the performance of the apparatus 700 and make helpful adjustments during operation.

The control buttons 705 may be strategically placed near the display screen 704, allowing for easy access and configuration of the device settings. Each button serves a specific function, such as setting the infusion rate, adjusting the volume of secondary medicine to be administered, or calibrating the sensing elements 711A. For example, a healthcare professional may use one of the control buttons 705 to program the apparatus 700 to deliver 5 mL of medication over a 10-minute period, facilitating precise dosing based on the patient's needs.

The power on/off switch 706 enables the operator to activate or deactivate the apparatus 700. This switch is designed for ease of use, with a clear indicator to show whether the device is currently powered on. For example, during emergency situations, the power on/off switch 706 allows for rapid activation of the apparatus 700 to initiate fluid delivery or infiltration detection immediately.

The apparatus 700 may be connected to the IV line 707 using a first hydraulic line 708 and a second hydraulic line 709, each equipped with a respective clamp, 708A and 709A. The first clamp 708A may be positioned at the proximal point 707A near the fluid source 208, while the second clamp 709A may be located at the distal point 707B near the cannula 210. These clamps can open, close, or restrict the IV line 707 as required, with the hydraulic lines 708 and 709 controlling their operation. For example, during the injection of the secondary medicine from the syringe 702, the first clamp 708A may close to block the flow of fluid 209 from the fluid source 208, while the second clamp 709A remains open to allow the secondary medicine to flow into the patient 211.

The hydraulic lines 708 and 709 may be integral to the operation of the clamps 708A and 709A. These lines deliver hydraulic pressure to the clamps 708A-709A, enabling precise control over their gripping strength. The hydraulic system may be managed by the controller within the housing 710, which adjusts the pressure based on the operational requirements. For example, during normal fluid administration, both clamps 708A-709A may remain partially open to allow a continuous flow of fluid 209 from the fluid source 208 to the patient 211. However, during a flushing operation or the administration of secondary medicine, the controller may temporarily increase the hydraulic pressure in the first hydraulic line 708 to tighten the grip of the first clamp 708A.

A sensing arm 711, positioned at a second connection point 707D on the IV line 707, contains sensing elements 711A that encircle the IV line. These sensing elements 711A are configured to detect various parameters related to the fluid flow within the IV line 707, such as pressure, flow rate, and acoustic signals. For example, a pressure sensor within the sensing elements 711A can identify sudden drops in pressure that may indicate infiltration or line dislodgement. Similarly, acoustic sensors can analyze the sound of fluid movement to detect irregularities, such as air bubbles or occlusions.

The placement of the sensing arm 711 at the second connection point 707D, below the first connection point 707C where the syringe 702 is attached, allows it to monitor the combined flow of primary and secondary fluids. This strategic positioning facilitates that any anomalies in the fluid mixture are detected before the fluid reaches the cannula 210. For example, if the secondary medicine from the syringe 702 does not mix properly with the primary fluid 209, the sensing elements 711A (using the controller) can alert the operator through the display screen 704.

The integration of clamps 708A and 709A, the syringe 702, and the sensing arm 711 within the apparatus 700 enables advanced functionalities that enhance patient safety and treatment efficacy. For example, the controller within the housing 710 can coordinate the operation of the clamps 708A-709A and the syringe 702 to perform automated flushing cycles, preventing blockages within the IV line 707. Additionally, the real-time feedback from the sensing elements 711A allows the apparatus 700 to detect potential infiltration risks and alert the operator promptly.

In some embodiments of the present invention, an apparatus for detecting IV infiltration and managing fluid delivery incorporates advanced sensing elements and communication capabilities to enhance operational precision and patient safety. These features include a piezoelectric sensor, ultrasonic flow meter, a sensing arm with adjustable configurations, and integration with centralized hospital monitoring systems.

The sensing arm may be equipped with sensing elements (e.g., 711A), which include a piezoelectric sensor and an ultrasonic flow meter. The piezoelectric sensor is configured to detect pressure variations within the IV line. For example, during normal fluid flow, the piezoelectric sensor measures consistent pressure levels; however, in cases of reverse pressure caused by blood reversal into the IV line, the sensor detects abnormal spikes or drops. Such reverse pressure may occur if the patient moves their hand or arm, causing temporary blockages or resistance in the IV line. The sensor immediately transmits this data to the controller, which analyzes it in real-time to determine if corrective action is required.

The ultrasonic flow meter, another useful component of sensing elements, measures the flow rate and direction of fluid within the IV line. It operates by emitting ultrasonic waves and analyzing their interaction with the fluid. For example, when fluid flows smoothly, the ultrasonic flow meter detects consistent wave patterns. However, irregularities, such as flow interruption due to hand movement or blockages, result in disrupted wave reflections, signaling potential issues. The controller processes these disruptions to assess the severity of the irregularity and trigger alerts or adjustments as needed.

The sensing arm may be configured to pivot or rotate around the IV line, allowing easy adjustment during setup or operation. This flexibility facilitates that the sensing elements are optimally positioned for accurate monitoring, regardless of the IV line's orientation or the patient's movement. For example, during setup, a medical professional can rotate the sensing arm to align it with the IV line's flow direction, facilitating unobstructed monitoring by the piezoelectric sensor and ultrasonic flow meter.

In response to detected irregularities, such as reverse pressure or disrupted flow patterns, the apparatus may generate a beep alert to notify medical personnel. For example, if a patient's hand movement causes a temporary blockage, the sensing elements detect the anomaly, and the apparatus emits an audible beep while transmitting detailed alert data to the integrated user interface or a connected external device. This real-time feedback allows for immediate intervention to restore proper flow.

The communication module within the apparatus is configured to interface with hospital information systems, facilitating centralized patient monitoring. For example, the module transmits data, including pressure trends, flow rates, and alerts, to a centralized system, enabling healthcare professionals to monitor multiple IV setups simultaneously. This integration facilitates seamless communication between the apparatus and hospital networks, improving workflow efficiency and enabling proactive responses to potential IV complications.

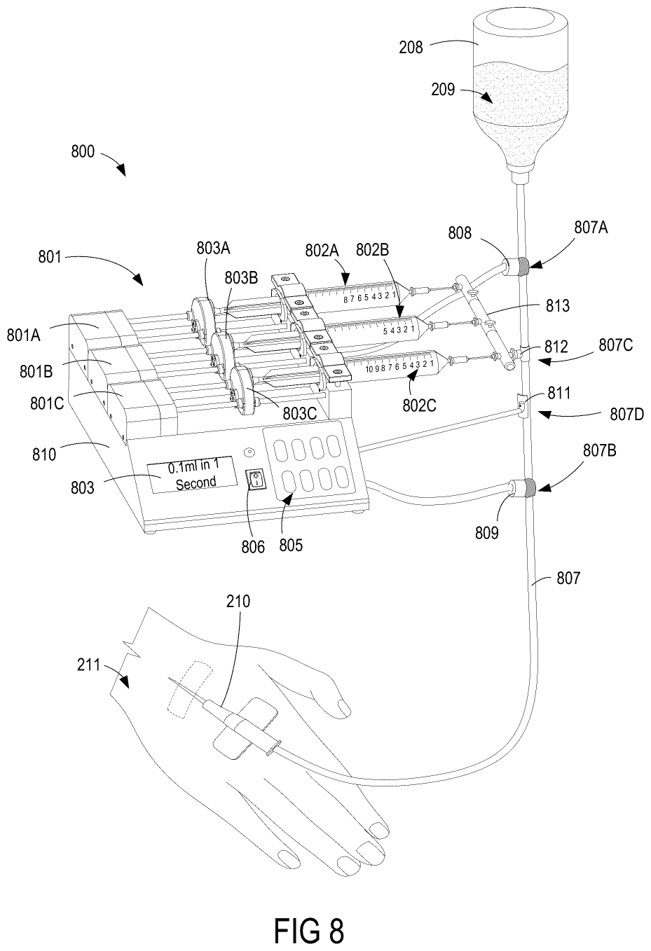

Referring now to FIG. 8, an apparatus 800 comprises an infusion pump 801 with one or more syringe holders configured to hold a plurality of syringes 802A-802C. Each syringe 802A-802C may be equipped with a respective plunger 803A-803C, which is connected to respective plunger controls 801A-801C. These plunger controls are designed to operate the syringes individually or collectively under the direction of the infusion pump 801. The apparatus 800 may further be integrated with an IV infiltration detection setup and connected to an IV line 807 for administering fluids 209 and monitoring flow conditions during intravenous therapy.

The infusion pump 801 may be designed to accommodate one or more syringes, providing enhanced flexibility in intravenous therapy management. Each syringe 802A-802C can contain a different medication or fluid, allowing for sequential, simultaneous, or emergency delivery as required. For example, syringe 802A may contain a saline solution for hydration, syringe 802B may hold an antibiotic, and syringe 802C may be designated for emergency medications such as epinephrine. This arrangement enables the apparatus 800 to address a wide range of clinical scenarios, from routine infusions to emergency interventions.

Each syringe 802A-802C may securely be held in its respective syringe holder, which is part of the infusion pump 801. The holders are constructed from durable medical-grade materials to maintain stability during operation. The plungers 803A-803C are connected to the respective plunger controls 801A-801C, which are motorized mechanisms capable of precise control over the movement of the plungers. For example, the plunger control 801A for syringe 802A can depress the plunger 803A at a predefined rate to deliver a consistent flow of fluid into the IV line 807. Similarly, the plunger control 801B for syringe 802B may operate at a different rate to administer medication over a longer duration.

The aggregation tube 813 plays a vital role in the operation of the apparatus 800. It collects fluid from the syringes 802A-802C and directs it to the IV line 807 at the first connection point 807C. This design allows multiple medications to be delivered through a single entry point (807C) in the IV line 807, reducing the complexity of the infusion setup. For example, during a multi-drug regimen, the aggregation tube 813 facilitates that fluids from all active syringes are mixed uniformly before entering the IV line 807. This feature may particularly be beneficial in critical care settings where multiple medications need to be administered simultaneously.

The connection to the IV line 807 may be facilitated by a Luer lock 812 at the first connection point 807C, providing a secure and leak-proof attachment. The IV line 807 may further be equipped with clamps and sensing elements, as described in earlier figures, for precise flow control and infiltration detection. The second connection point 807D, equipped with a sensing arm 811 containing sensing elements 811A, monitors the combined flow from the syringes and primary fluid source before the fluid reaches the cannula 210. This placement allows the sensing elements 811A to detect anomalies such as inconsistent flow rates, air bubbles, or pressure drops, providing real-time feedback to the medical professional.

The plunger controls 801A-801C allow for independent operation of each syringe. This independence enables diverse infusion strategies, such as staggered administration or priority-based delivery. For example, syringe 802A can be programmed to deliver 5 mL of saline over a period of 10 minutes, while syringe 802B administers 10 mL of an antibiotic over a period of 20 minutes. Meanwhile, syringe 802C may remain on standby as an emergency syringe, preloaded with a life-saving medication that can be activated remotely by a medical professional when an alert is received.

The emergency activation feature of the apparatus 800 adds an extra layer of functionality. For example, in a scenario where the patient exhibits signs of anaphylaxis, a medical professional can remotely activate syringe 802C via the communication modules integrated into the apparatus. This feature may particularly be valuable in home care or remote settings, where immediate physical intervention by a medical professional may not be feasible. The controller within the infusion pump 801 coordinates this activation, so that the emergency medication is delivered directly and promptly through the IV line 807.

The controller within the infusion pump 801, similar to the one described in FIG. 3A, manages the operation of all components, including the plunger controls 801A-801C, clamps, and sensing elements. The controller may be equipped with a processor and an AI engine to execute predefined infusion protocols, analyze real-time sensor data, and adapt to changing conditions. For example, if the sensing elements 811A detect a sudden pressure drop indicative of infiltration, the controller can pause all ongoing infusions, close the clamps, and alert the operator via the display screen 810 or a connected device.

The display screen 810, mounted on the front panel of the infusion pump 801, provides a real-time interface for monitoring and controlling the device. It displays important information such as the status of each syringe, current infusion rates, and sensor readings from the IV line 807. The display screen 810 may also show alerts or recommendations based on data analyzed by the AI engine. For example, if the fluid level in syringe 802B is nearing depletion, the display screen 810 can prompt the operator to refill or replace the syringe.

The control buttons 806 located below the display screen 810 allow the operator to configure the apparatus 800. Each button corresponds to a specific function, such as selecting the active syringe, adjusting infusion parameters, or initiating a flushing cycle. For example, a healthcare professional may use the control buttons 806 to set syringe 802A to deliver 1 mL/min of saline while programming syringe 802B to administer an antibiotic at a slower rate of 0.5 mL/min.

The hydraulic clamps 808A and 809A, positioned at the proximal point 807A and distal point 807B of the IV line 807 respectively, regulate the flow of fluid 209 from the fluid source 208 and the aggregation tube 813. These clamps can open or close the IV line 807 based on commands from the controller. For example, during the administration of a secondary medication from syringe 802A, the controller may close hydraulic clamp 808A to temporarily block fluid from the primary source, allowing the medication to flow uninterrupted to the patient 211.

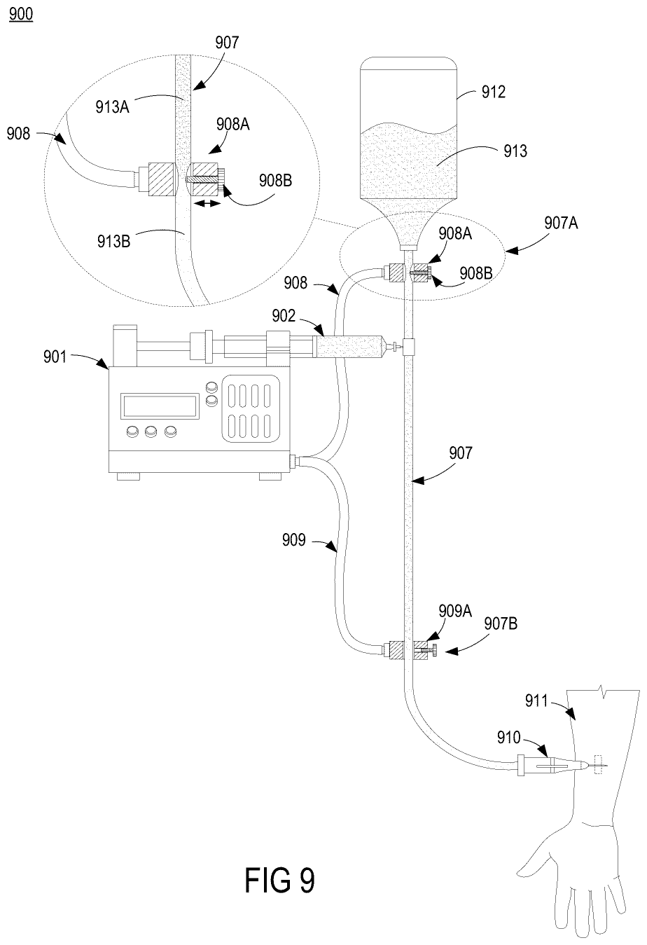

Referring now to FIG. 9, an exemplary system 900 is depicted, illustrating the working mechanism of the clamps integrated within the apparatus 901 for IV infiltration detection and infusion control. The system 900 may be connected to an IV line 907 for administering a fluid 913 from a fluid source 912 to a patient's arm 911 through a cannula 910. The apparatus 901 integrates an infusion pump with a syringe 902 and a sophisticated IV infiltration detection setup. This embodiment showcases how the apparatus 901 employs its clamps to control and regulate the flow of the fluid 913 through the IV line 907, while also monitoring for infiltration and other abnormalities.

The apparatus 901, designed to house both infusion and detection functionalities, may include a syringe 902 that may deliver secondary medication into the IV line 907. The syringe 902 may be controlled via a plunger system, as described in previous figures, and works in conjunction with clamps 908A and 909A to manage fluid flow through the IV line 907. The syringe 902 may also be used for flushing the IV line 907 or introducing a secondary fluid for therapeutic purposes. The apparatus 901 may include controllers and sensors to facilitate seamless integration of these functionalities.

The IV line 907 may be connected to the fluid source 912 at its proximal point 907A and to the cannula 910 at its distal point 907B. The IV line 907 facilitates the flow of the fluid 913, such as saline, medication, or nutrients, to the patient 911. The fluid source 912 may be a bottle or pouch containing the fluid 913, which is introduced into the IV line 907 through the first hydraulic line 908. The distal end of the IV line 907 connects to the cannula 910, which is inserted into a vein in the patient's arm 911 for fluid delivery.

The clamps 908A and 909A play an important role in controlling the flow of fluid 913 through the IV line 907. The first clamp 908A may be positioned at the proximal point 907A, near the fluid source 912, while the second clamp 909A may be located at the distal point 907B, near the cannula 910. Both clamps are capable of opening, closing, or partially compressing the IV line 907 to regulate fluid flow. The operation of these clamps may be automatically controlled by the apparatus 901 through hydraulic lines 908 and 909, which transmit hydraulic pressure to adjust the clamp positions.

The hydraulic lines 908 and 909 are flexible tubes that connect the clamps 908A and 909A to the apparatus 901. These hydraulic lines transmit pressure generated by a hydraulic system within the apparatus 901, allowing the clamps to be precisely adjusted. For example, during the administration of a secondary fluid from the syringe 902, the apparatus 901 may increase the pressure in the first hydraulic line 908 to close the first clamp 908A, thereby stopping the flow of fluid 913 from the fluid source 912. Simultaneously, the hydraulic line 909 may maintain or reduce pressure to keep the second clamp 909A open, allowing the secondary fluid to flow freely into the IV line 907.

The exploded view of the first clamp 908A in FIG. 9 illustrates how the clamp (one or both—908A-909A) interacts with the IV line 907 to control fluid flow. The first clamp 908A comprises a movable component, which compresses the IV line 907 to varying degrees. The semi-compressed state of the first clamp 908A, as shown in the exploded view, reduces the quantity of fluid 913 flowing through the IV line 907. Specifically, the fluid flow may be reduced from a first quantity 913A to a second quantity 913B. This controlled reduction in fluid flow may be required in scenarios such as administering a specific medication dose, preventing infiltration, or responding to a remote command from a medical professional.

The manual control mechanism of the first clamp 908A, represented by the screw 908B, provides an alternative means of regulating fluid flow. The screw 908B can be tightened or loosened to adjust the compression of the IV line 907. For example, in emergency scenarios where the hydraulic system is non-operational, the screw 908B can be used to manually close or open the IV line 907, facilitating continuous operation of the system. The dual control options-hydraulic and manual-enhance the versatility and reliability of the clamp mechanism.

The fluid control scenarios demonstrated by FIG. 9 include various use cases where precise regulation of fluid flow is important. For example, during an infusion therapy session, the clamps 908A and 909A can dynamically adjust the flow rate of the fluid 913 based on real-time feedback from the sensing elements (e.g., 811 shown in FIG. 8) in the apparatus 901. This adjustment may be initiated by the apparatus 901 itself or in response to remote commands from a medical professional. For example, if an air bubble is detected in the IV line 907, the apparatus 901 may close the first clamp 908A to prevent the bubble from reaching the patient 911.

The integration of the hydraulic system within the apparatus 901 enables advanced control over the clamps 908A-909A. The hydraulic system may be managed by a controller within the apparatus 901, which calculates the required pressure levels based on sensor inputs and predefined infusion protocols. For example, if the sensing elements detect a high flow rate at the distal point 907B, the controller may increase the hydraulic pressure in the first hydraulic line 908 to tighten the first clamp 908A, thereby reducing the flow rate.

The sensors and controllers within the apparatus 901 enhance its functionality and safety. These components monitor various parameters such as fluid pressure, flow rate, and the presence of air bubbles. The data collected by the sensors is processed by the controller, which adjusts the operation of the clamps 908A-909A and syringe 902 accordingly. For example, if the sensors detect a sudden drop in pressure, indicating potential infiltration, the controller may pause the infusion and alert the operator via the display screen on the apparatus 901 or remotely.

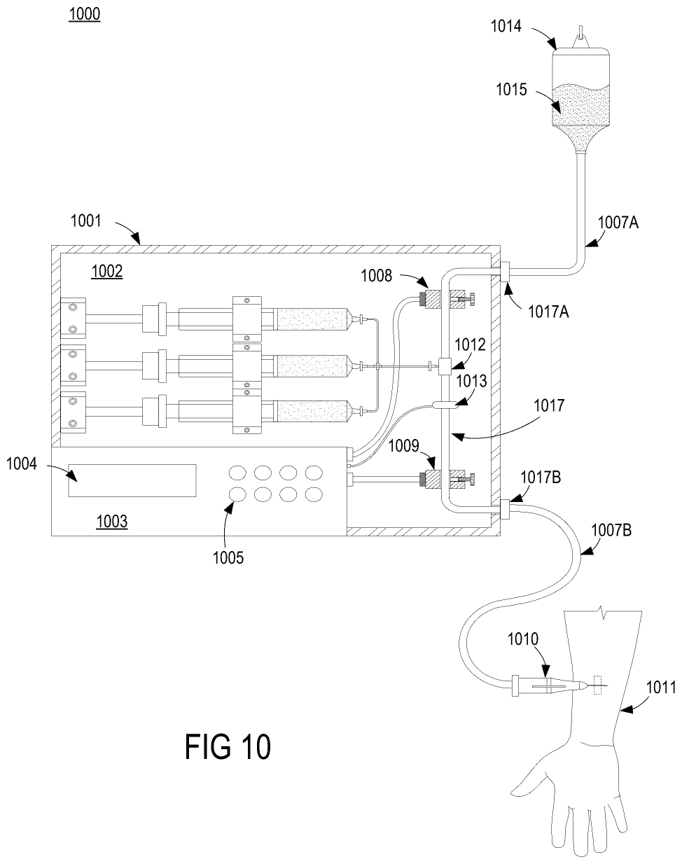

Referring now to FIG. 10, an exemplary system 1000 is illustrated showcasing an apparatus 1001 designed for IV infiltration detection and precise control of fluid delivery through an internal fluid flow line 1017. The apparatus 1001 operates by integrating various components, including ports, clamps, sensing elements, and syringe holders, with the internal fluid flow line 1017 to facilitate safe and accurate fluid administration to a patient 1011. This detailed depiction demonstrates how the fluid 1015 from the fluid source 1014 is processed through the apparatus 1001 for delivery to the patient 1011 via the lower IV line 1007B and cannula 1010 connected to the patient 1011.

The internal fluid flow line 1017 represents a sealed conduit within the apparatus 1001, designed to handle the passage of fluid 1015 from an input port 1017A to an output port 1017B of the apparatus 1001. The input port 1017A may be connected to the upper IV line 1007A, which leads to the fluid source 1014. The fluid source 1014 may be a saline bag, a nutrient pouch, or a medication reservoir. The output port 1017B may be connected to the lower IV line 1007B, which ultimately delivers the processed fluid 1015 to the patient 1011 through a cannula 1010 or similar setup.

The internal fluid flow line 1017 may further be enhanced with multiple components to allow precise control and monitoring of fluid flow. For example, the apparatus 1001 incorporates a Luer lock 1012 that facilitates the connection of one or more syringes housed in the syringe holders 1002. This connection allows secondary fluids or medications to be introduced into the internal fluid flow line 1017 for subsequent delivery to the patient. The Luer lock 1012 provides a secure and seamless integration between the syringes and the internal fluid flow line 1017, thereby maintaining sterility and preventing leaks during operation.

The syringe holders 1002 are configured to securely accommodate syringes of varying sizes and capacities, enabling flexibility in administering different volumes of secondary medication. Each syringe within the syringe holders 1002 may be connected to a common or respective motorized plunger control system, which allows for controlled injection of the medication into the internal fluid flow line 1017. The integration of these syringes with the internal fluid flow line 1017 via the Luer lock 1012 permits a seamless transition of secondary fluids into the IV line 1007 and ultimately to the patient 1011.

The clamps 1008 and 1009 may be strategically placed along the internal fluid flow line 1017 to provide precise control over the fluid flow. The first clamp 1008 may be positioned near the input port 1017A at a proximal end, while the second clamp 1009 may be located near the output port 1017B at a distal end. These clamps may be operated by a hydraulic or mechanical control system (as described in FIG. 9) within the apparatus 1001. For example, the first clamp 1008 can be tightened to temporarily halt the flow of fluid 1015 from the fluid source 1014, while the second clamp 1009 can be adjusted to regulate the outflow of fluid to the patient 1011. This dual-clamp system facilitates that the fluid flow remains consistent and within desired parameters.

The sensing elements 1013, positioned along the internal fluid flow line 1017 between the first clamp 1008 and the second clamp 1009, provide real-time monitoring of the fluid characteristics. These sensing elements 1013 may include pressure sensors, flow rate detectors, and air bubble detectors. For example, a pressure sensor can detect fluctuations in the fluid pressure, which may indicate blockages or infiltration. A flow rate detector can monitor the speed of fluid delivery, so that the prescribed rate is maintained. Additionally, air bubble detectors can identify the presence of air in the fluid line, which can be a potential risk for the patient.

The controller 1003, housed within the apparatus 1001, serves as the central processing unit for managing the operation of the various components. The controller 1003 receives data from the sensing elements 1013 and processes this information to adjust the operation of the clamps 1008 and 1009, the syringe holders 1002, and other connected systems. For example, if the sensing elements 1013 detect an air bubble, the controller 1003 may temporarily close the first clamp 1008 to prevent the bubble from advancing toward the patient 1011.

The display 1004 provides an intuitive interface for medical professionals to view real-time data and control the apparatus 1001. The display 1004 may show parameters such as fluid flow rate, pressure, and the status of the syringes. Alongside the display, the apparatus may include control buttons 1005, which allow the user to manually adjust settings such as the flow rate, clamp positions, and syringe operation. These controls provide a high degree of flexibility and adaptability, making the apparatus suitable for various medical scenarios.

The fluid source 1014, connected to the apparatus 1001 via the upper IV line 1007A, is a useful component of the system 1000. This fluid source 1014 may contain a variety of fluids, such as saline, glucose, or specialized medications. The apparatus 1001 may be designed to accommodate different types of fluid sources, facilitating compatibility with a wide range of medical treatments. The fluid 1015 is drawn from the fluid source 1014 into the internal fluid flow line 1017 through the input port 1017A.

The cannula 1010, connected to the lower IV line 1007B, serves as the point of delivery for the fluid 1015 to the patient 1011. This setup allows the fluid to be administered directly into the patient's bloodstream, facilitating efficient absorption and therapeutic effect. The positioning of the clamps 1008 and 1009 along the internal fluid flow line 1017 so that the fluid is delivered at the correct rate and pressure, preventing complications such as infiltration or over-infusion.

The integration of the components within the apparatus 1001 highlights its versatility and functionality. The internal fluid flow line 1017, clamps 1008 and 1009, sensing elements 1013, and syringe holders 1002 work in harmony to provide a comprehensive solution for IV infiltration detection and fluid administration. The apparatus 1001 may be capable of adapting to various medical scenarios, making it an invaluable tool for healthcare providers.

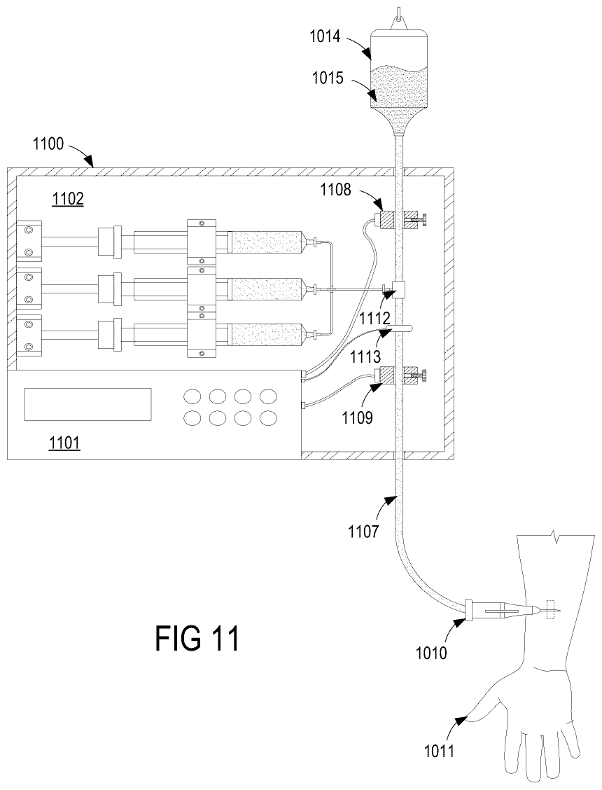

Referring now to FIG. 11, an exemplary apparatus 1100 is illustrated, wherein the IV line 1107 of an IV setup is configured to pass directly through the apparatus 1100 rather than being externally connected, as depicted in FIG. 10. The apparatus 1100 is an enclosed system designed to facilitate both infusion and IV infiltration detection while maintaining a streamlined connection with the IV setup. The apparatus 1100 comprises several components, including a controller 1101, an infusion pump 1102, clamps 1108 and 1109, a Luer lock 1112, and sensing elements 1113, all housed within a protective casing to optimize operational efficiency and patient safety.

The IV line 1107 may be inserted through the apparatus 1100 using an accessible panel or entry point in the casing. This configuration allows the IV line 1107 to pass internally through the various operational components of the apparatus 1100, including the upper clamp 1108, the lower clamp 1109, the Luer lock 1112, and the sensing elements 1113. Such an arrangement eliminates the need for external tubing connections, reducing potential leakage points and maintaining a secure and sterile environment. For example, during setup, a medical professional may open a hinged panel on the apparatus 1100, insert the IV line 1107 through a designated channel, and secure it within the clamps 1108-1109 and other components.

The upper clamp 1108, positioned near the entry point of the IV line 1107, is designed to control the flow of fluid 1015 from the fluid source 1014, connected via the upper IV line 1007A. The upper clamp 1108 may utilize hydraulic or motorized actuation to apply variable pressure to the IV line 1107, thereby regulating or halting fluid flow as needed. For example, if a medical professional determines that the flow rate of the fluid 1015 needs adjustment, the controller 1101 can modify the pressure exerted by the upper clamp 1108 to achieve the desired flow rate. The hydraulic line associated with the upper clamp 1108 may include a fine-tuning mechanism to allow precise adjustments.

The lower clamp 1109, positioned near the exit point of the IV line 1107, serves a complementary function by controlling the outflow of fluid 1015 to the patient 1011 through the lower IV line 1007B and cannula 1010. The lower clamp 1109 may similarly be equipped with hydraulic or motorized actuation, enabling the apparatus 1100 to modulate fluid delivery based on the patient's requirements. For example, during the administration of a secondary medication, the lower clamp 1109 may be partially or fully closed to prevent backflow or to regulate the entry of the medication into the patient's vein.

The Luer lock 1112, situated between the clamps 1108 and 1109 along the IV line 1107, provides a secure connection point for secondary medications delivered through the infusion pump 1102. The infusion pump 1102, comprising multiple syringe holders, allows the apparatus 1100 to administer precise volumes of medication via syringes connected to the Luer lock 1112. Each syringe within the infusion pump 1102 may be independently controlled by the controller 1101, enabling the apparatus to accommodate various treatment protocols. For example, one syringe may deliver a steady dose of saline solution while another syringe is reserved for emergency medication, which can be activated remotely by a medical professional.

The sensing elements 1113, integrated along the IV line 1107 between the Luer lock 1112 and the lower clamp 1109, are configured to monitor key parameters such as pressure, flow rate, and the presence of air bubbles within the fluid 1015. These sensors provide real-time data to the controller 1101, which processes the information to detect potential issues such as infiltration, occlusion, or irregular flow. For example, a pressure sensor may detect a sudden increase in resistance, indicating a potential infiltration event, while a flow sensor may identify a reduction in flow rate caused by partial occlusion.

The controller 1101 acts as the central processing unit of the apparatus 1100, managing the operation of the clamps 1108 and 1109, the infusion pump 1102, and the sensing elements 1113. The controller 1101 may execute software algorithms to analyze data from the sensing elements 1113, generate alerts for medical professionals, and automate the operation of the clamps and pump. For example, if the sensing elements 1113 detect air bubbles in the fluid 1015, the controller 1101 may temporarily close the upper clamp 1108 to prevent the bubbles from reaching the patient 1011, while simultaneously issuing an alert through the display screen or a connected device.

The display screen within the controller 1101 provides a user-friendly interface for monitoring and adjusting the apparatus 1100. The screen may display real-time metrics such as flow rate, pressure, and syringe status, allowing medical professionals to make informed decisions. Additionally, the control buttons associated with the display enable manual overrides and adjustments, offering flexibility in treatment protocols. For example, a user may adjust the pressure exerted by the clamps 1108 and 1109 or select the volume of medication to be administered by a specific syringe.

The fluid source 1014, containing the fluid 1015, may be connected to the apparatus 1100 via the upper IV line 1007A. This source may include various types of fluids, such as saline, glucose, or specialized medications, depending on the treatment requirements. The apparatus 1100 may be designed to accommodate different fluid types, providing compatibility with a wide range of medical applications. The connection between the fluid source 1014 and the apparatus 1100 is secured to maintain sterility and prevent contamination.

The cannula 1010, attached to the lower IV line 1007B, delivers the fluid 1015 from the apparatus 1100 to the patient 1011. The integration of the clamps 1108 and 1109, the Luer lock 1112, and the sensing elements 1113 within the apparatus 1100 facilitates that the fluid 1015 is delivered safely and efficiently. The internal configuration of the IV line 1107 within the apparatus 1100 eliminates the need for external tubing connections, reducing the risk of leaks and enhancing the system's reliability.

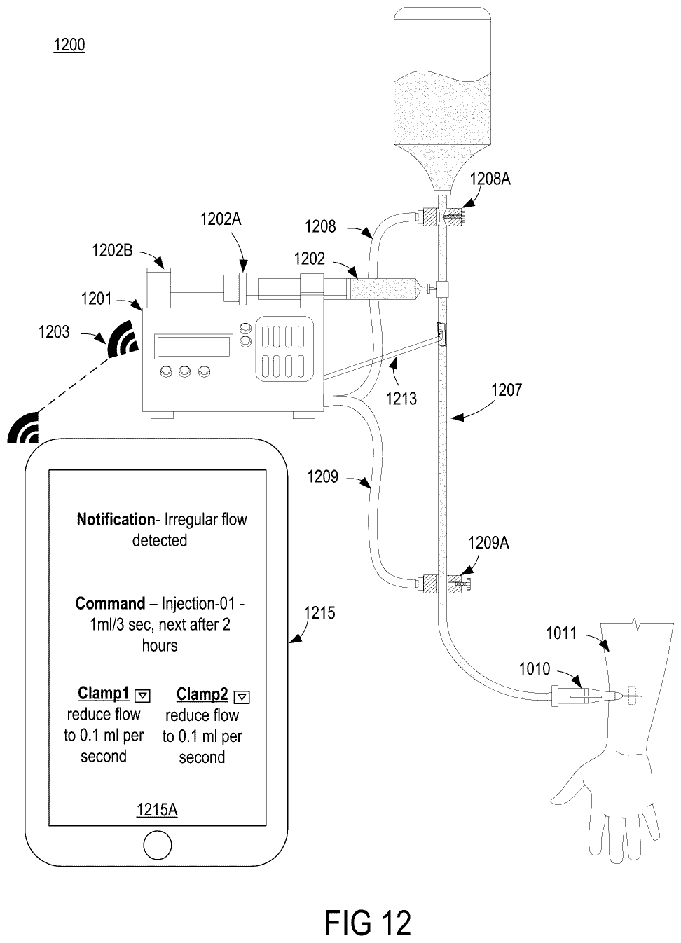

Referring now to FIG. 12, an exemplary system 1200 is illustrated wherein the apparatus 1201 integrates communication capabilities to facilitate remote control and monitoring by a medical professional. The apparatus 1201 is operatively connected to an IV setup comprising an IV line 1207, fluid source 1014 containing fluid 1015, and a cannula 1010 inserted into the vein of a patient 1011. The system 1200 allows for dynamic control of fluid flow, administration of secondary medications via an infusion syringe 1202, and IV infiltration detection using sensing mechanisms integrated into the system. Control commands are transmitted through a remote communication device, such as a smartphone 1215, via a wireless network established through the Wi-Fi module 1203.

The apparatus 1201 may include a plunger-controlled infusion syringe 1202, connected to the IV line 1207 at a position between the proximal end at 1208A and the distal end at 1209A. The syringe plunger 1202A of the infusion syringe 1202 may be actuated using a plunger control mechanism 1202B, which operates automatically based on pre-programmed settings or commands received remotely. For example, if a medical professional needs to deliver an emergency dose of medication from the infusion syringe 1202, a remote command transmitted via the smartphone 1215 can trigger the plunger control mechanism 1202B to compress the syringe plunger 1202A and inject the medication into the IV line 1207.

The Wi-Fi module 1203 integrated into the apparatus 1201 facilitates wireless communication with the smartphone 1215. This connection enables bidirectional data exchange, allowing the apparatus 1201 to transmit real-time notifications and receive operational commands. For example, if the sensing line 1213 (using sensing elements) detects an abnormality such as irregular fluid flow or increased resistance within the IV line 1207, the apparatus 1201 can send an alert notification to the smartphone 1215. The notification displayed on the smartphone 1215, such as “Irregular flow detected,” may include detailed information about the issue and suggested corrective actions.

The sensing line 1213, connected to the IV line 1207 between the first clamp 1208A and the second clamp 1209A, comprises advanced sensors capable of detecting various anomalies. These sensors may measure parameters such as fluid pressure, flow rate, and the presence of air bubbles. For example, a pressure sensor within the sensing line 1213 may identify an increase in resistance indicative of IV infiltration or occlusion. Similarly, a flow sensor may detect an irregular flow pattern that deviates from the programmed settings of the apparatus 1201. These data points are analyzed by the internal controller of the apparatus 1201, which subsequently triggers appropriate notifications or corrective actions.

The first clamp 1208A and second clamp 1209A, controlled by respective hydraulic lines 1208 and 1209, provide precise regulation of fluid flow through the IV line 1207. For example, upon receiving a remote command from the smartphone 1215, the first clamp 1208A may be tightened to restrict fluid flow from the fluid source 1014, allowing for the administration of secondary medication from the infusion syringe 1202. Conversely, the second clamp 1209A may be opened to facilitate the unobstructed delivery of medication to the patient 1011 through the cannula 1010. The ability to remotely control these clamps offers significant advantages in scenarios where immediate intervention is required, such as during a suspected infiltration event.

The smartphone 1215, as shown in FIG. 12, serves as the interface for remote monitoring and control. Notifications displayed on the smartphone 1215, such as “Irregular flow detected,” include actionable options for the medical professional. For example, the interface 1215A may allow the user to adjust the flow rate by setting the first clamp 1208A and second clamp 1209A to specific positions. Additionally, commands to activate the infusion syringe 1202 for emergency medication delivery can be executed directly from the smartphone 1215. The integration of these capabilities facilitates prompt response by medical professionals to any irregularities in the IV setup.

The fluid flow monitoring mechanism within the apparatus 1201 leverages real-time data from the sensing line 1213 to facilitate optimal fluid administration. For example, if the fluid 1015 from the fluid source 1014 encounters resistance due to partial occlusion in the IV line 1207, the apparatus 1201 can adjust the hydraulic lines 1208 and 1209 to correct the issue. Additionally, the infusion syringe 1202 may be activated to supplement the fluid delivery with medication that reduces resistance or alleviates the patient's condition.

The controller within the apparatus 1201 orchestrates the operation of all connected components, including the clamps 1208A and 1209A, the infusion syringe 1202, and the sensing line 1213. This controller is programmed to analyze sensor data, execute pre-set infusion schedules, and respond to remote commands. For example, if an air bubble is detected within the IV line 1207, the controller may temporarily close the first clamp 1208A to prevent the bubble from advancing, while simultaneously notifying the medical professional to take corrective action.

The emergency scenarios may also be addressed effectively using the system 1200. For example, during a sudden drop in patient blood pressure due to medication side effects, the medical professional can remotely activate the infusion syringe 1202 to deliver a counteracting drug. The clamps 1208A and 1209A can be adjusted to prioritize the administration of this medication over the regular IV fluid 1015. Such functionality highlights the system's adaptability to dynamic medical conditions.

The infusion syringe 1202 and its plunger control mechanism 1202B may particularly be advantageous for precise medication delivery. For example, a time-sensitive drug requiring a specific infusion rate can be administered without manual intervention, as the plunger control mechanism 1202B regulates the syringe plunger 1202A based on the programmed settings. Additionally, the system 1200 allows for customizable schedules where different medications can be administered at specific intervals, enhancing the efficiency of patient care.

The integration of multiple communication channels, such as Wi-Fi 1203, Bluetooth, and cellular connectivity, further extends the functionality of the apparatus 1201. In areas with limited Wi-Fi coverage, the system can switch to cellular networks to maintain communication with the medical professional's smartphone 1215. This redundancy provides uninterrupted operation in various settings, including remote or rural healthcare facilities.

In some embodiments, the medical professional may utilize the smartphone 1215 to remotely send commands to the apparatus 1201 to activate the infusion syringe 1202 for delivering a precise quantity of medicine. For example, the professional may input parameters such as a delivery volume of 5 mL, a timing interval of 2 seconds, and a force equivalent to 50 mmHg pressure. These commands are received via the Wi-Fi module 1203 of the apparatus 1201, enabling the controller within the apparatus 1201 to execute the instructions. The plunger control mechanism (1202B) of the infusion syringe 1202 adjusts accordingly to deliver the specified volume and pressure through the IV line 1207. The system may log the delivery event and provide real-time feedback on the smartphone 1215, such as “5 mL medicine delivered at 50 mmHg in 2 seconds,” facilitating seamless monitoring and adjustments, as necessary, during IV therapy.

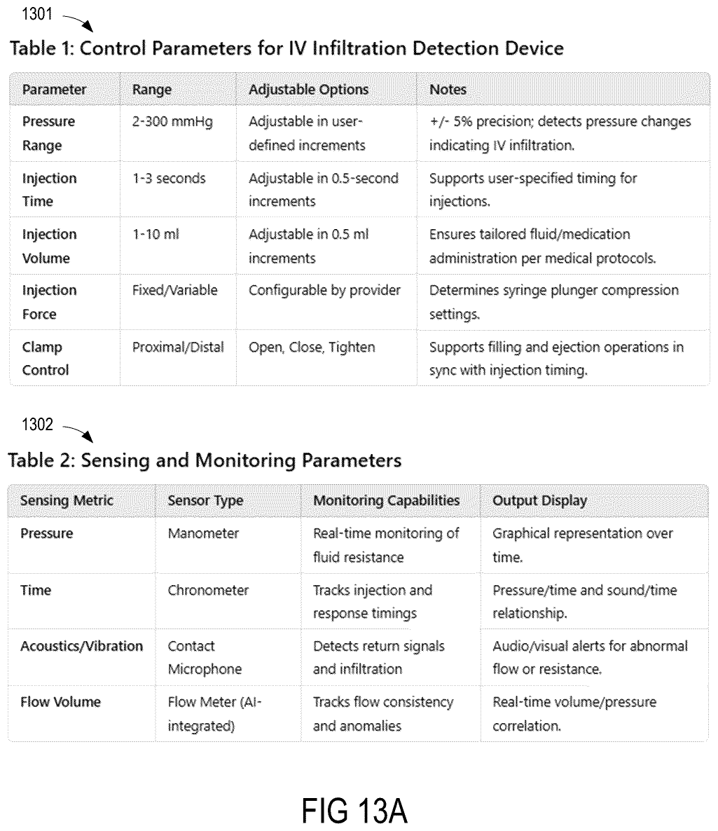

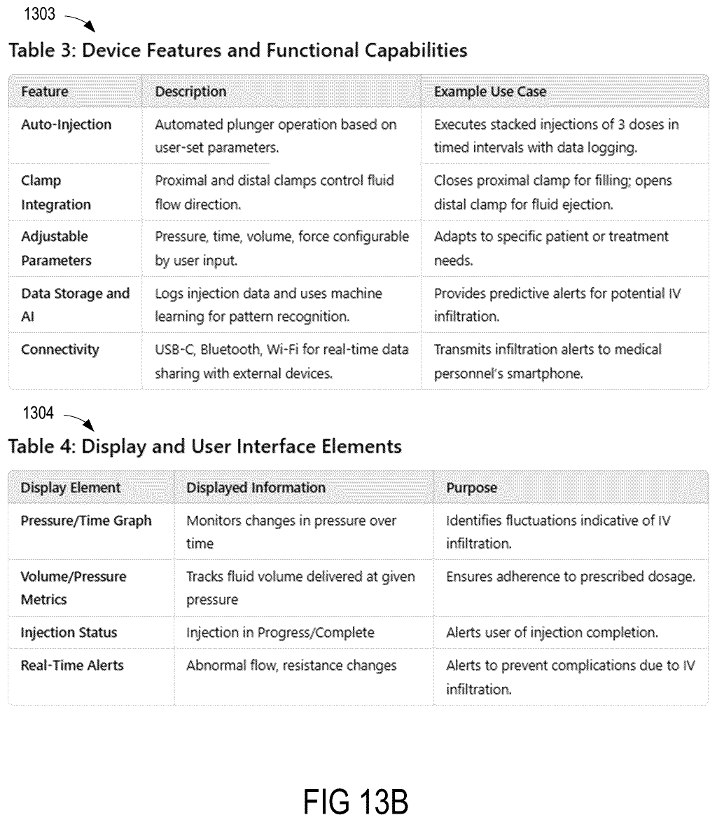

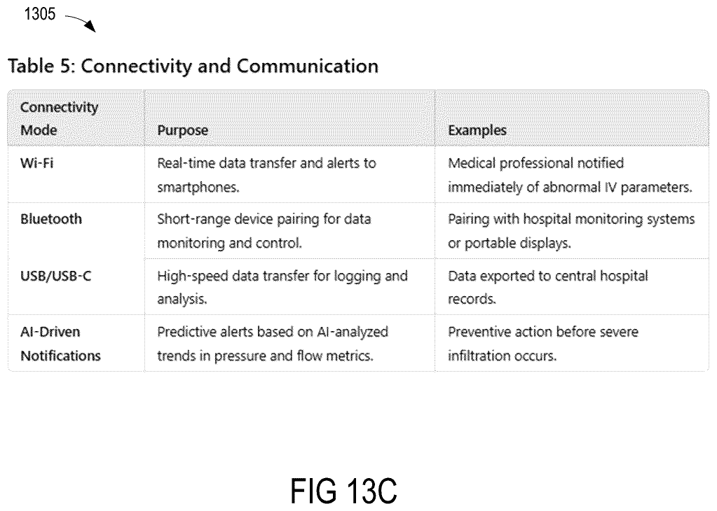

Referring now to FIGS. 13A-13C, these figures illustrate exemplary tables 1301-1302 that provide detailed insights into various control parameters, sensing mechanisms, operational features, and data outputs for the IV infiltration detection device. The depiction of tables 1301-1302 aims to elucidate the functional and technical aspects of the apparatus (described in various embodiments of the present invention), highlighting the precision and customization capabilities that align with its intended purpose of detecting IV infiltration and managing infusion operations.

Table 1301, titled Control Parameters for IV Infiltration Detection Device, outlines key adjustable parameters and their corresponding configurations, providing insight into the device's operational flexibility. The parameters listed may include Pressure Range, Injection Time, Injection Volume, Injection Force, and Clamp Control. For example, the Pressure Range, adjustable between 2-300 mmHg, reflects the device's ability to monitor and modulate pressure with a precision of +/−5%. This functionality may particularly be advantageous in detecting subtle pressure variations within the IV line (e.g., pressure spikes indicating infiltration). The integration of user-defined increments enables medical professionals to tailor the pressure settings to specific patient requirements or infusion protocols. For example, during the administration of sensitive medications, maintaining a lower pressure threshold can prevent fluid leakage into surrounding tissues.

The Injection Time, adjustable in increments as small as 0.5 seconds within a range of 1-3 seconds, facilitates precise timing for infusion processes. This time variability can accommodate scenarios where rapid fluid administration is required, such as during emergency treatments. For example, an anti-inflammatory drug might need to be delivered quickly to counteract swelling in an acute situation, which the system can achieve by configuring the injection timing accordingly. In some cases, a medicine dose (e.g., 5 ml) may be delivered over a longer duration, such as 1-2 hours, for treatments requiring sustained and gradual infusion, like administering chemotherapy drugs or hydration fluids to ensure controlled absorption by the patient. Additionally, in certain scenarios, a 5 ml dose may be delivered in steps, such as administering 1 ml over 2 seconds initially, followed by 0.5 ml after an hour, with each step taking 1-3 seconds to inject, to align with specific therapeutic protocols or patient tolerance levels.

The Injection Volume, ranging from 1 to 10 ml with 0.5 ml increments, provides fine- grained control over the quantity of fluid or medication being infused. This adjustable volume mechanism may particularly be useful for pediatric patients, where lower volumes are required due to their smaller body sizes. Additionally, the ability to set specific volumes can be advantageous in administering doses of high-potency drugs that require precise measurements.

Injection Force, described as configurable by the provider, supports either fixed or variable configurations to match the clinical needs. For example, medications with higher viscosity, such as chemotherapy agents, may require greater injection force, while saline infusions may utilize lower forces. The ability to adapt this parameter provides compatibility with a diverse range of medications and fluid consistencies.

The Clamp Control, supporting operations such as open, close, and tighten, reflects the apparatus's ability to manage fluid flow through the IV line. For example, during the filling phase of a syringe, the proximal clamp may remain open while the distal clamp is tightened to facilitate fluid draw from the IV source. Conversely, during the ejection phase, the distal clamp opens while the proximal clamp closes to direct fluid flow toward the patient. This feature not only enhances operational control but also minimizes the risk of backflow or unintended leakage.

The control parameters outlined in Table 1301, including pressure range, injection time, injection volume, injection force, and clamp control, may be remotely adjusted by a medical professional using wireless commands transmitted to the apparatus. The communication module of the apparatus, which may include connectivity options such as Wi-Fi, Bluetooth, or cellular networks, facilitates this functionality by allowing real-time interaction between the medical professional's external device, such as a smartphone or tablet, and the apparatus. For example, a medical professional can remotely configure the injection volume to 5 ml in precise increments of 0.5 ml, set the injection timing to deliver the fluid over a specific duration (e.g., 2 seconds), or regulate the pressure to match therapeutic requirements within a range of 2-300 mmHg. Additionally, commands to open, close, or tighten the proximal and distal clamps can be issued, enabling the professional to dynamically control fluid delivery or temporarily halt the flow in response to real-time patient feedback or system alerts, thereby enhancing the flexibility and precision of IV therapy.

Table 1302, titled Sensing and Monitoring Parameters, provides an overview of the device's sensing capabilities and their respective outputs. The table lists metrics such as Pressure, Time, Acoustics/Vibration, and Flow Volume, along with their associated sensor types and monitoring capabilities. The Manometer, for example, enables real-time monitoring of fluid resistance, with graphical representations displayed over time. This feature helps to identify early signs of infiltration, as sudden changes in resistance can indicate fluid leaking into surrounding tissues.

The Chronometer, used to track injection and response times, facilitates adherence to prescribed infusion protocols. For example, the ability to measure and compare injection times across multiple administrations can help medical professionals identify deviations or inconsistencies in the delivery process, potentially highlighting issues such as blockages or patient movement affecting the IV setup.

The Contact Microphone, which detects return signals from the IV line, adds an acoustic monitoring layer to the device's capabilities. This feature may particularly be useful for identifying irregularities such as backflow or air bubbles in the IV line. For example, during or after an injection, the device may detect abnormal vibrations caused by resistance or obstruction in the line, prompting an alert to the medical professional.

The Flow Meter, integrated with AI, enhances the device's monitoring capabilities by detecting flow consistency and anomalies. The real-time visualization of flow metrics, such as pressure and volume, provides actionable insights for maintaining optimal infusion conditions. For example, during an infusion, the flow meter can identify gradual reductions in flow rate, which may indicate partial infiltration or line kinking, allowing timely interventions.

Both tables 1301-1302 highlight the device's capacity for automation and customization, emphasizing its adaptability to various clinical scenarios. For example, a scenario involving a pediatric patient may require low-pressure settings, minimal injection volumes, and high-frequency acoustic monitoring to avoid tissue damage. Conversely, an adult patient undergoing high-dose chemotherapy might benefit from higher pressure thresholds, larger injection volumes, and enhanced flow monitoring to facilitate efficient drug delivery.

Table 1303 outlines the features and functional capabilities integrated into the IV infiltration detection device, which enable precision, flexibility, and automation in its operation. Each feature is designed to address specific clinical challenges while providing a user-friendly interface for medical professionals. Below are detailed explanations, examples, and embodiments for each feature listed in the table.

Auto-Injection: The automated plunger operation allows user-defined injection parameters such as force, timing, and sequence. For example, this feature can execute stacked injections of three doses within specified time intervals, facilitating consistent drug administration. An embodiment of this functionality may include its use during emergency care, where a medical professional can program the device to administer epinephrine or other required medications at specific intervals without requiring manual input. This feature may particularly be useful in high-pressure environments, such as intensive care units, where time is of the essence.

Clamp Integration: Proximal and distal clamps are incorporated to control the direction of fluid flow, closing the IV line during filling and opening it for ejection. For example, during syringe filling, the proximal clamp opens while the distal clamp closes, preventing backflow. In an alternative embodiment, the clamps may be operated remotely through wireless communication, enabling a nurse to control fluid flow adjustments without being physically present at the patient's bedside. This may particularly be useful for telemedicine setups in remote healthcare facilities.

Adjustable Parameters: The device allows users to configure parameters such as pressure, time, and volume. For example, the injection volume can be adjusted in increments of 0.5 ml up to 10 ml, providing precision for both pediatric and adult patients. An embodiment may involve administering microdoses of insulin for diabetic patients, where the adjustable parameters facilitate that the correct dosage is delivered without error. The time for injections, adjustable between 1 to 3 seconds, may also be configured for treatments requiring slow and steady medication delivery.

Data Storage and Analysis: The device utilizes machine learning algorithms to analyze injection data, identifying patterns that may indicate potential IV infiltration. For example, if the device detects repeated resistance spikes during infusion, it may predict the likelihood of infiltration and alert the medical staff. In one embodiment, this feature may be integrated with hospital records to compile a patient's infusion history, helping medical professionals optimize future treatments.

Connectivity: The device supports USB-C, Bluetooth, and Wi-Fi for seamless data transfer. For example, it can transmit real-time injection data to a centralized monitoring system, enabling physicians to review multiple patients' data simultaneously. An embodiment may involve integrating the device with a smartphone application that notifies doctors of anomalies, such as irregular fluid flow, allowing for prompt corrective actions.

Table 1304 highlights the display elements and user interface features designed to provide real-time feedback and facilitate intuitive device operation. Each element facilitates accurate monitoring and timely interventions during IV therapy. Below are detailed explanations, examples, and embodiments for each feature.

Pressure/Time Graph: This display element visually represents changes in pressure over time, aiding in the identification of fluctuations that may indicate IV infiltration. For example, a sudden drop in pressure during an infusion may signal that the IV line has become dislodged. In one embodiment, the device may highlight abnormal pressure trends in red on the graph, immediately drawing the attention of medical staff. This feature may particularly be useful during long-duration infusions, where constant monitoring is required.

Volume/Pressure Metrics: This element tracks the volume of fluid delivered at specific pressure levels. For example, the device may display that 5 ml of fluid has been delivered at a pressure of 50 mmHg, helping medical professionals evaluate the infusion's efficiency. In an alternative embodiment, the device may generate a real-time comparison between actual and expected volume/pressure metrics, alerting the user to deviations that may require adjustments.

Injection Status: The injection status indicator provides real-time updates on the injection process, such as whether an injection is in progress or completed. For example, during chemotherapy, the status indicator may notify the nurse when a syringe is empty and needs to be replaced. In one embodiment, the status may be accompanied by audible alerts, such as beeps or voice notifications, enhancing its accessibility in busy medical environments.

Real-Time Alerts: This display element notifies users of abnormal flow conditions or resistance changes detected during infusion. For example, if the sensing elements detect increased resistance, the device may display an alert stating, “High Resistance Detected-Check IV Line.” An embodiment of this feature may include customizable alert settings, allowing medical professionals to specify the type and frequency of notifications they wish to receive, so that important alerts are prioritized.

Table 1305 describes various connectivity modes incorporated into the device and their functionalities, with corresponding examples. These connectivity mechanisms enhance data transfer, remote monitoring, and integration with external systems to improve device operation and usability. The following discussion elaborates on each connectivity mode with examples and embodiments.

In some embodiments of the present invention, Wi-Fi connectivity is configured to enable real-time data transfer and alert mechanisms. For example, the device may detect abnormal IV parameters, such as a sudden change in pressure or flow resistance, and transmit a notification to a medical professional's smartphone. The alert may provide actionable information, such as specific IV line parameters requiring adjustment. In another embodiment, Wi-Fi may be used for integration with centralized hospital monitoring systems, enabling medical personnel to observe and manage multiple devices simultaneously from a remote station. This can be particularly advantageous in scenarios requiring close monitoring, such as intensive care units or emergency response situations.

In some embodiments, the device may utilize Bluetooth connectivity for short-range pairing with external systems or devices. For example, the device can be paired with a portable monitoring tablet used by medical personnel during patient rounds. This allows localized control of infusion parameters and viewing of real-time data metrics, such as pressure trends or acoustic signals. An additional embodiment may involve the device communicating directly with bedside monitoring systems via Bluetooth to facilitate uninterrupted operation in environments with limited Wi-Fi access, such as field clinics or ambulatory care units.

In another embodiment, USB or USB-C ports may be included for high-speed data transfer to external systems. For example, the device may store historical data, including pressure measurements, flow rates, and acoustic analysis, which can then be exported to a centralized database or electronic health records (EHR) system. In some embodiments, the USB-C interface may allow simultaneous data transfer and device charging, supporting continuous use in hospital environments. For example, clinicians conducting post-procedure evaluations can retrieve logged data from the device for analysis, enabling detailed documentation of IV infiltration trends.

In some embodiments, the device may employ AI-driven notifications to enhance its predictive capabilities. For example, the device may analyze historical data using machine learning algorithms to identify patterns indicative of potential IV infiltration before it occurs. If irregularities are detected, the system can generate alerts recommending preventive measures, such as adjusting the flow rate or inspecting the IV line for obstructions. An embodiment may involve integrating these AI-driven alerts with hospital communication systems, automatically escalating critical cases to senior medical staff for immediate intervention. For example, the system may predict infiltration risks based on small but consistent fluctuations in pressure and notify relevant personnel to take corrective action.

### Examples and Embodiments

Wi-Fi Connectivity: In a surgical setting, Wi-Fi enables the device to relay real-time updates to an anesthesiologist's dashboard, providing continuous feedback on IV parameters during critical operations.

Bluetooth Pairing: In outpatient care, the device pairs with a nurse's handheld tablet, allowing localized control without requiring direct interaction with the IV setup.

USB/USB-C Integration: During a clinical trial, USB-C ports are used to download anonymized patient data for large-scale statistical analysis of IV infiltration rates.

AI Predictive Alerts: For patients undergoing chemotherapy, the device learns individual infusion patterns and alerts staff when deviations indicate potential infiltration, preventing complications.

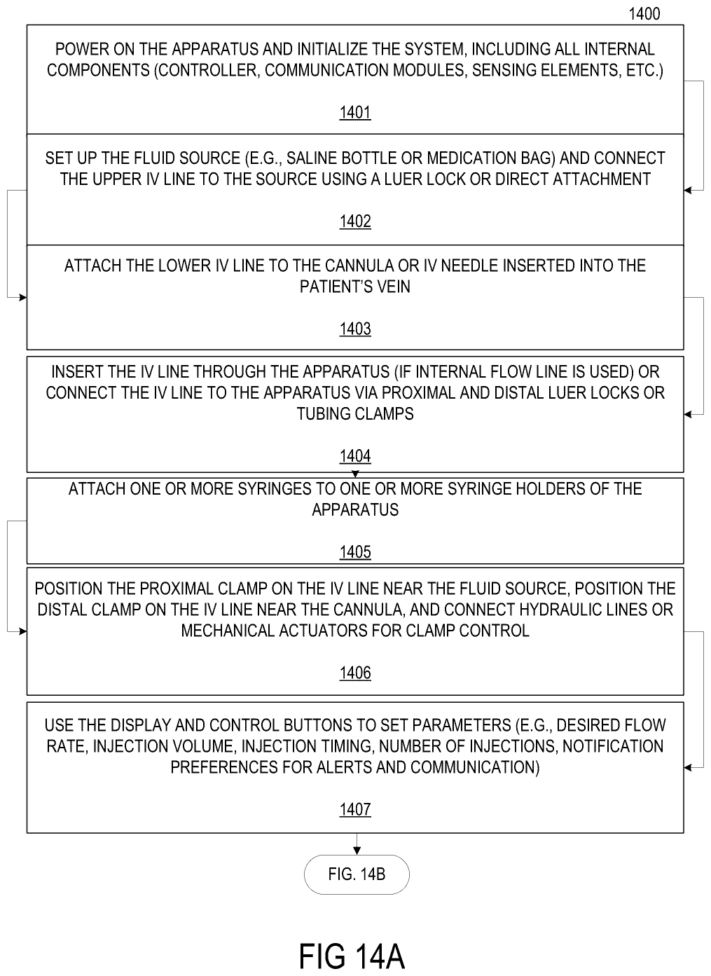

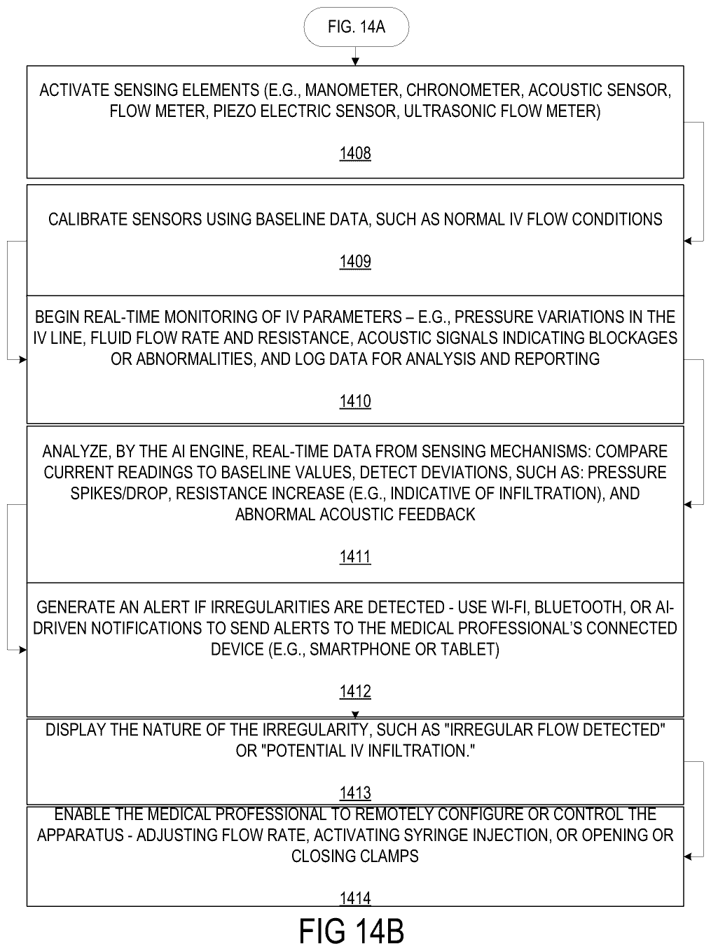

Referring now to FIGS. 14A-14B, an exemplary flowchart 1400 is illustrated, detailing the comprehensive sequence of steps 1401-1414 involved in the operation and utilization of the IV infiltration detection device, as described in the present disclosure. The flowchart 1400 provides a methodical overview of the apparatus's functionality, starting from initialization and setup to integration with an IV line, configuration of operational parameters, and active monitoring for IV infiltration or irregularities. The depicted steps 1401-1414, encompass the exemplary aspects of the invention, including powering on the apparatus, connecting the IV line to the fluid source and the patient, integrating the apparatus with the IV setup, attaching syringes for secondary medication administration, positioning clamps for fluid control, and configuring the system using an intuitive interface. This structured methodology allows for precise monitoring and control of IV therapy, enhancing patient safety and enabling real-time interventions. Each step is designed to optimize device functionality and adaptability, incorporating advanced features such as sensor integration, hydraulic clamp control, and user-configurable settings, thereby addressing the diverse needs of medical professionals in a clinical or remote setting.

At step 1401, the process begins by powering on the apparatus and initializing all its internal components. These components include the controller (e.g., a microprocessor integrated with an AI engine), communication modules (e.g., Wi-Fi, Bluetooth, USB/USB-C), sensing elements, and syringe actuation mechanisms. Initialization involves booting up the software programs embedded in the controller, which prepares the system for operation. During this phase, diagnostic checks may also be conducted to verify the functionality of individual components, such as clamps, sensors, and the syringe holder mechanisms.

For example, during initialization, the sensing elements may conduct a baseline calibration of pressure and flow parameters in the IV line, so that any deviations during operation are detected with precision. The apparatus may also perform a self-check for any malfunctions or connectivity issues in its communication modules. For example, if the device relies on Wi-Fi for transmitting alerts to a medical professional, this module is activated and connected to the designated network at this step. Embodiments of this initialization may also include a user interface that displays the operational readiness of the system and highlights any detected issues.

At step 1402, the IV setup process commences by connecting the upper end of the IV line to the fluid source, such as a saline solution bag or medication bottle. This connection is facilitated by a Luer lock or another standard medical connection mechanism. The fluid source contains the primary fluid (e.g., saline, dextrose, or medication) to be delivered into the patient's bloodstream. This step is fundamental to establishing the flow of fluid into the IV line.

An example of this setup is when a saline bag is hung on an IV pole, and the upper end of the IV line is securely connected to its outlet port using a Luer lock. The connection must be precise and secure to prevent any leaks or air ingress into the line. Embodiments of the invention may include an accessory attachment mechanism that enhances the ease of connecting the IV line to the fluid source, such as a snap-fit or click-lock design. Additionally, sensors within the apparatus may begin detecting the pressure and flow characteristics of the fluid at this stage, which helps establish baseline conditions for subsequent monitoring.

At step 1403, the lower end of the IV line is connected to the cannula or IV needle inserted into the patient's vein. This step involves securing the distal end of the IV line to the cannula, facilitating an uninterrupted pathway for fluid delivery from the source to the patient. The connection should maintain sterility to prevent infections and must be secure to avoid dislodgment during operation.

For example, in one embodiment, the distal end of the IV line is connected using a Luer lock compatible with standard cannulas. In more advanced setups, the apparatus may include a sensing mechanism at this connection point to monitor for infiltration or flow resistance changes. This may include acoustic or pressure sensors that detect abnormalities, such as fluid leaking into surrounding tissues instead of entering the vein. Moreover, the system may log this connection as a starting point for tracking the patient's IV therapy session.

At step 1404, the IV line is either inserted through the apparatus (if the device features an internal fluid flow line) or connected externally using proximal and distal Luer locks or tubing clamps. This step facilitates that the apparatus integrates seamlessly with the IV setup, establishing a pathway for fluid to flow through the apparatus for monitoring and control.

For example, if the apparatus features an internal flow line, the IV line is threaded through clamps, sensors, and Luer locks housed within the device. Alternatively, if the system uses external connections, the proximal and distal clamps are secured around the IV line near their respective positions. Embodiments of this step may include adjustable clamps or Luer locks designed to accommodate IV lines of various diameters, enhancing compatibility. Additionally, a sensor calibration mechanism may activate at this point, establishing initial readings for fluid flow and pressure.

At step 1405, one or more syringes are attached to one or more syringe holders within the apparatus. The syringes may contain secondary medication or fluid to be administered alongside the primary IV fluid. These syringes are equipped with plungers connected to a control mechanism, allowing precise control over the timing, volume, and pressure of injections.

For example, a single syringe may contain a dose of antibiotics to be administered intermittently, while another syringe holds a saline flush solution. The syringe holders are designed to accommodate syringes of various sizes and capacities, such as 5 ml, 10 ml, or 20 ml. In one embodiment, the apparatus may include a mechanism to automatically prime the syringe, facilitating that no air is present in the fluid pathway before injection. Advanced embodiments may also feature an automated syringe recognition system that detects the type and volume of the attached syringe, simplifying the setup process.

At step 1406, the proximal clamp is positioned on the IV line near the fluid source, and the distal clamp is positioned near the cannula. These clamps are controlled by hydraulic lines or mechanical actuators integrated into the apparatus. The positioning and control of these clamps are vital for regulating fluid flow through the IV line, particularly during filling or ejection operations.

For example, during the filling of a syringe, the proximal clamp may remain open while the distal clamp is closed, creating a controlled vacuum effect that draws fluid into the syringe. Conversely, during injection, the distal clamp opens while the proximal clamp closes, facilitating that the fluid is directed toward the patient. Embodiments may include advanced clamp designs with variable compression levels, allowing fine-tuned control over fluid flow and pressure. Sensors embedded within the clamps may also provide real-time feedback on their status, such as whether they are fully open, partially closed, or fully closed.

At step 1407, the user configures the apparatus by using the display and control buttons to set operational parameters. These parameters include flow rate, injection volume, timing, and the number of injections. The display provides a real-time interface for inputting and confirming these settings, while the control buttons or a touchscreen interface facilitate user interaction.

For example, the user may set an injection volume of 5 ml to be delivered over a duration of 2 seconds, with the system automatically adjusting the plunger speed to achieve the desired flow rate. In another embodiment, the display may show a graphical representation of the expected pressure curve for the selected parameters, providing additional insight into the injection dynamics. Advanced embodiments may also allow remote configuration via a connected smartphone or tablet, enabling medical professionals to adjust settings without being physically present at the apparatus.

At step 1408, the sensing elements of the apparatus, such as a manometer, chronometer, acoustic sensor, flow meter, piezoelectric sensor, and ultrasonic flow meter, are activated to prepare for the monitoring of various parameters of the IV line. These sensing elements are integrated into the system to measure key variables, such as pressure, time, flow rate, resistance, and acoustic feedback within the IV line. The activation process involves the initiation of power supply to the sensors and establishing communication with the controller within the apparatus. For example, the manometer may detect pressure variations, while the piezoelectric sensor may identify minute vibrations caused by IV infiltration. The activation facilitates that the sensors are functioning optimally and ready to collect real-time data. This step is significant in laying the foundation for subsequent data collection and analysis by the system.

In some embodiments, the activation process may involve diagnostic checks to confirm the proper functioning of each sensor. For example, a baseline reading of ambient conditions may be taken to verify the calibration status of the sensors. If a malfunction is detected in any sensor during activation, the system may display an alert on the apparatus display or send a notification to a connected device, prompting the medical professional to address the issue. Such diagnostic capabilities enhance the reliability of the apparatus and minimize the risk of inaccurate monitoring.

At step 1409, the sensors are calibrated using baseline data, such as normal IV flow conditions. Calibration is helpful to set a reference point for the system to identify deviations in IV parameters accurately. For example, the system may record the pressure, flow rate, and acoustic signals under normal operating conditions without any blockages or infiltration. This baseline data is stored in the apparatus's memory or cloud-based storage and serves as a comparison metric during real-time monitoring.

In one embodiment, the calibration process involves the infusion of a saline solution through the IV line at a predetermined flow rate and pressure. The sensing elements record these values and create a profile representing normal conditions. If the system is being used in a new environment or with a different IV setup, the calibration process can be repeated to adapt to specific requirements. For example, if a pediatric patient requires lower flow rates, the baseline data can be adjusted accordingly.

At step 1410, real-time monitoring of IV parameters begins. The system continuously tracks variables such as pressure variations in the IV line, fluid flow rate, resistance, and acoustic signals. The collected data is logged and stored for further analysis and reporting. For example, if the pressure within the IV line begins to rise unexpectedly, it may indicate a blockage or infiltration. Similarly, variations in acoustic feedback detected by the piezoelectric sensor may suggest an irregularity in fluid flow.

In some embodiments, the system may use a flow meter to detect the exact rate at which fluid is being infused into the patient. Any significant deviation from the calibrated baseline, such as a sudden drop in flow rate, triggers a detailed analysis by the AI engine within the apparatus. This real-time monitoring capability may particularly be useful in high-risk scenarios where rapid detection of anomalies is necessary to prevent complications.

At step 1411, the system generates an alert if irregularities are detected in the IV setup. The alert may be communicated via Wi-Fi, Bluetooth, or AI-driven notifications to the medical professional's connected device, such as a smartphone or tablet. For example, if the system identifies a sharp increase in resistance within the IV line, it may send an alert stating, “Potential IV infiltration detected—Immediate attention required.” This alert system allows medical professionals to respond promptly to potential issues, minimizing patient risk.

In certain embodiments, the alert may include detailed information about the irregularity, such as the exact location of the blockage or the time and nature of the pressure deviation. The medical professional can use this information to make informed decisions, such as adjusting the infusion rate or replacing the IV line. Additionally, the system may log the alert for future reference, allowing for comprehensive documentation of the patient's treatment.

At step 1412, the nature of the irregularity is displayed on the apparatus, such as “Irregular flow detected” or “Potential IV infiltration.” The display provides a clear and concise summary of the issue, enabling medical professionals to understand the situation at a glance. For example, the display may show a graphical representation of the pressure over time, highlighting any significant deviations from the baseline. This visual aid assists in diagnosing the problem and determining the appropriate course of action.

In some embodiments, the display may include additional metrics, such as the exact flow rate, resistance values, and acoustic signal patterns. These details allow for a more comprehensive analysis of the IV setup's condition. For example, if the system detects a blockage near the distal end of the IV line, the display may highlight this specific area, helping the medical professional pinpoint the issue quickly.

At step 1413, the medical professional is enabled to remotely configure or control the apparatus using the connected device. This capability allows for adjustments such as modifying the flow rate, injection volume, or timing of the syringe. For example, if the system detects an irregularity in the IV setup, the medical professional can send a command to increase the flow rate temporarily to clear a minor blockage. Similarly, if a patient requires an immediate injection of medication, the professional can activate the syringe remotely.

In one embodiment, the connected device displays a control interface that allows the user to select specific actions, such as opening or closing the clamps or initiating an emergency injection. The system's AI engine may also provide recommendations based on real-time data, further assisting the medical professional in making informed decisions. This remote-control capability may particularly be valuable in scenarios where immediate intervention is required but the medical professional is not physically present.

In some embodiments of the present invention, the apparatus is configured to allow precise control and adjustment of proximal and distal clamps integrated within the IV setup. These clamps are used to control the fluid flow through the IV line during different operational modes, such as syringe injection or syringe filling. For example, the proximal clamp, positioned closer to the fluid source, and the distal clamp, positioned closer to the patient, can be adjusted independently to facilitate various operational requirements.

To perform syringe injection, the apparatus commands the proximal clamp to close, thereby restricting the flow of fluid from the fluid source into the IV line. Simultaneously, the distal clamp is opened, allowing the fluid or medication within the syringe to be pushed through the IV line and into the patient's vein. This precise control of the clamps is achieved through hydraulic or electromechanical actuators controlled by the apparatus's controller. For example, the hydraulic pressure within the actuators can be modulated to achieve the desired clamp position, facilitating consistent and effective delivery of the injected fluid. In another embodiment, stepper motors may be employed to precisely control the tightening or loosening of the clamps.

Conversely, during the syringe filling operation, the proximal clamp is opened to allow fluid from the source to enter the syringe, while the distal clamp is closed to prevent backflow or leakage into the IV line. This configuration enables the syringe to be filled efficiently without disrupting the existing fluid flow in the IV line. The apparatus's controller may determine the duration and extent of the clamp adjustments based on preconfigured settings or real-time data analysis, such as the current flow rate, pressure, or volume of the fluid.

In some embodiments, the apparatus employs advanced sensors to analyze post-injection signals within the IV line. These sensors, which may include acoustic sensors, flow meters, and pressure transducers, monitor the IV line for return signals or anomalies that may indicate issues such as incomplete injection, infiltration, or patient discomfort. For example, an acoustic sensor may detect irregularities (acoustic resistance) in the sound waves traveling through the fluid, while a flow meter can measure variations in flow consistency. The data collected from these sensors is logged and analyzed to evaluate the success of the injection and the patient's response.

The apparatus's AI engine may process the logged data to identify trends or anomalies that may require further adjustments to the injection parameters or clamp positions. For example, if a post-injection analysis reveals inconsistent flow rates or unusual resistance in the IV line, the system can notify the medical professional and recommend specific corrective actions, such as repeating the injection or modifying the flow rate for subsequent operations.

The apparatus is designed to enable continuous monitoring and adjustments throughout the IV therapy process. Sensors within the system continuously track IV parameters such as pressure, flow rate, and acoustic feedback. Based on this real-time data, the controller can dynamically adjust the clamp positions or syringe operations to maintain optimal conditions. For example, if a blockage is detected in the IV line, the system may automatically close the distal clamp and open the proximal clamp to flush the blockage while maintaining patient safety.

In some embodiments, the apparatus supports the export of logged data to external systems for further analysis, storage, or regulatory reporting. The data, including injection volumes, flow rates, and pressure profiles, can be transferred via USB, USB-C, or Wi-Fi connectivity. For example, medical professionals can use this data to generate reports for patient records or compliance with regulatory standards. The exported data may also be analyzed using external software to refine treatment protocols or identify long-term trends in patient responses to IV therapy.

Once the IV therapy process is complete, the apparatus facilitates an orderly shutdown. Monitoring is stopped, the clamps are deactivated, and the apparatus is disconnected from the IV line. This step prevents unnecessary wear on the system components and prepares the apparatus for maintenance and reuse.

In embodiments where the apparatus may include integrated UV sanitization, the internal components such as the clamps, internal fluid line, and syringe holder are sanitized automatically. The UV sanitization system may include UV-C lamps strategically positioned to irradiate all internal surfaces exposed to fluid contact, effectively eliminating any residual pathogens. In scenarios where manual sterilization is required, the apparatus is designed for easy disassembly, allowing healthcare professionals to thoroughly clean and sterilize each component. For example, the syringe holder may be detachable, and the clamps may feature quick-release mechanisms for simplified cleaning.

These embodiments demonstrate the apparatus's comprehensive functionality in controlling and monitoring IV therapy, while also addressing the practical aspects of maintenance and data management to support reliable and efficient operation.

### Conclusion

A number of embodiments of the present disclosure have been described. While this specification contains many specific implementation details, there should not be construed as limitations on the scope of any disclosures or of what may be claimed, but rather as descriptions of features specific to particular embodiments of the present disclosure.

In the present disclosure, certain reference numbers are consistently used across multiple figures to represent corresponding or similar components within the invention. This approach has been adopted to streamline the description, minimize redundancy, and enhance clarity for ease of understanding. For example, reference numbers such as those denoting the fluid source, fluid, the apparatus, IV line, cannula, and patient are consistently employed across different figures to denote their respective elements and functionalities. While the configurations or interactions of these components may vary in specific embodiments, the consistent use of reference numbers enables a cohesive narrative and facilitates a seamless interpretation of the invention's features across various illustrations. Such consistent labeling does not limit the scope or applicability of the invention but rather enhances the comprehensibility of the figures and associated descriptions.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, separation of various system components in the embodiments described above should not be understood as requiring such separation in all embodiments, and it should be understood that the described program components and systems can generally be integrated together in a single hardware and/or software product or packaged into multiple products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. A method of detecting a state of intravenous (IV) infiltration using an apparatus, the method comprising the steps of:
a. connecting an IV line installed in a patient to a primary input connection of the apparatus, the apparatus being configured to detect the state of IV infiltration;
b. connecting the IV line to an output connection of the apparatus, the output connection facilitating fluid delivery from a fluid source to the patient through the IV line;
c. placing one or more syringe holders upon a secondary input connection of the apparatus, the secondary input connection operably linked to an infusion mechanism within the apparatus;
d. staging the one or more syringe holders to hold one or more springs in one of a compression or expansion state, the one or more springs being operably configured to regulate fluid injection;
e. placing one or more syringes containing one or more fluid solutions within the respective one or more syringe holders, the one or more syringes operably associated with the one or more springs for pressurization;
f. monitoring at least one parameter selected from pressure, temperature, acoustic resistance, or flow rate with one or more sensors of the apparatus;
g. releasing an engagement arm on the one or more syringe holders to activate the one or more springs, thereby pressurizing the one or more fluid solutions within the one or more syringes and driving the one or more fluid solutions into the IV line;
h. recording the at least one parameter monitored by the one or more sensors, the at least one parameter being stored in a data storage module within the apparatus for subsequent analysis;
i. processing the monitored at least one parameter with a processor integrated within the apparatus, the processing comprising analyzing the at least one parameter for deviations indicative of IV infiltration, blockages, or abnormalities in fluid flow through the IV line; and
j. providing feedback to a user based upon the processing of the monitored at least one parameter.

2. The method of claim 1, wherein the apparatus transmits the feedback to an external device via a communication module integrated into the apparatus.

3. The method of claim 2, wherein the apparatus further comprises a display configured to visually present the monitored at least one parameter in real-time.

4. The method of claim 1, wherein the one or more sensors comprise a pressure sensor configured to detect changes in pressure indicative of IV infiltration or blockages.

5. The method of claim 1, wherein the one or more sensors comprise a fluid flow sensor configured to detect fluid flow irregularities in the IV line.

6. The method of claim 1, wherein the one or more sensors comprise a temperature sensor configured to detect deviations in fluid temperature.

7. The method of claim 1, wherein the processor is configured to utilize an artificial intelligence (AI) engine to analyze the monitored at least one parameter and identify patterns indicative of IV infiltration.

8. The method of claim 1, wherein the one or more syringe holders are configured to hold syringes of varying sizes.

9. The method of claim 1, wherein the one or more springs in the one or more syringe holders are adjustable to regulate a force applied during fluid injection.

10. The method of claim 1, wherein the apparatus further comprises a proximal clamp and a distal clamp, each of the proximal clamp and the distal clamp operably connected to the IV line, one or both of the proximal clamp and the distal clamp being configured to selectively occlude fluid flow through the IV line.

11. An apparatus for detecting a state of intravenous (IV) infiltration in an IV setup, the apparatus comprising:
a. a controller comprising a processor and an artificial intelligence (AI) engine, configured to execute software instructions, process data, analyze real-time and historical IV parameters, and control various components of the apparatus;
b. a communication module operably connected to the controller, configured to transmit and receive data via one or more communication interfaces to enable remote monitoring, alerts, and control of the apparatus;
c. a data storage module configured to store sensor data, operational parameters, and logged injection events, and timestamps for analysis and record-keeping;
d. a sensing arm, being attachable to an IV line, comprising one or more sensors to detect one or more parameters indicative of one or more of IV infiltration, blockages, or abnormalities in fluid flow through the IV line;
e. a first clamp, operably connected to the controller and being attachable on a proximal point of the IV line near a fluid source, the first clamp being configured to selectively occlude fluid flow during syringe filling using an infusion pump attached to the IV line or in response to detected irregularities in the IV line; and
f. a second clamp, operably connected to the controller and being attachable on a distal point of the IV line near a cannula attached to a patient;
wherein the controller executes software instructions to analyze real-time data from the sensing arm and determines whether the detected one or more parameters indicate IV infiltration, blockages, or abnormalities in fluid flow, and thereby controls one or more of: the first clamp, the second clamp, and the sensing arm.

12. The apparatus of claim 11, further comprises one or more syringe holders configured to securely hold one or more syringes of varying sizes, each syringe operably connected to an aggregation tube which is further connected to the IV line through a Luer lock.

13. The apparatus of claim 12, further comprises a plunger control mechanism, operably connected to the controller, configured to regulate a movement of a syringe plunger of the one or more syringes for fluid injection, the plunger control mechanism being adjustable for variable injection volumes and timings.

14. The apparatus of claim 13, further comprises an internal fluid flow line, connected to an input port and an output port of the apparatus, the input port being configured to connect to the IV line from the fluid source and the output port being configured to connect to the IV line leading to the patient, wherein each of the sensing arm, the first clamp, and the second clamp are internally attachable to the internal fluid flow line instead of the IV line.

15. The apparatus of claim 14, further comprises a UV sanitization system, integrated within the apparatus, configured to sanitize the internal fluid flow line, the one or more syringe holders, the one or more syringes, the first clamp, and the second clamp before or after use.

16. The apparatus of claim 11, further comprising a display means with a user interface, the display means operably connected to the controller, configured to:
i. display real-time data, including pressure, flow rate, and temperature;
ii. allow a user to configure operational parameters, such as injection volume, timing, and clamp operations; and
iii. provide visual alerts and notifications upon detecting IV infiltration or abnormalities in the IV line.

17. The apparatus of claim 11, wherein the one or more sensors in the sensing arm include an acoustic sensor configured to detect sound wave patterns indicative of air bubbles or irregular fluid flow within the IV line.

18. The apparatus of claim 11, wherein the one or more sensors in the sensing arm include a temperature sensor configured to detect deviations in fluid temperature, such deviations being analyzed by the controller for abnormal flow conditions.

19. The apparatus of claim 11, wherein the communication module is configured to send predictive alerts to an external device of a medical professional, the predictive alerts being generated based on trends analyzed by the AI engine in the controller.

20. The apparatus of claim 11, wherein the display means further comprises a touchscreen interface for direct user interaction with the apparatus.
